(19) United States
(12) Patent Application Publication
(10) Pub. No.: US 2023/0381170 A1
(43) Pub. Date: Nov. 30, 2023

(54) METHOD FOR TREATING ALS/FTD THROUGH DEGRADATION OF RNA REPEAT EXPANSION

(71) Applicant: University of Florida Research Foundation, Incorporated, Gainesville, FL (US)

(72) Inventor: Matthew D. Disney

(73) Assignee: University of Florida Research Foundation, Incorporated, Gainesville, FL (US)

(21) Appl. No.: 18/032,648

(22) PCT Filed: Oct. 18, 2021
(86) PCT No.: PCT/US2021/055408

§ 371 Date: Apr. 19, 2023

Related U.S. Application Data
(60) Provisional application No. 63/094,117, filed on Oct. 20, 2020.

(51) Int. Cl. A61K 31/475; A61K 47/54; A61K 47/60; A61P 25/28

(52) U.S. Cl. CPC A61K 31/475; A61K 47/545; A61K 47/60; A61P 25/28

(57) ABSTRACT

Described are small molecule embodiments, ALS compounds, that bind with the (G₄C₂)^{exp} RNA repeat transcription of the chromosome 9 open reading frame 72 involved in amyotrophic lateral sclerosis and frontotemporal dementia (ALS/FTD). These ALS compounds comprise a pyridocarbazole moiety having at least one substituent. Preferred ALS compounds comprise bridged dimers of the pyridocarbazole moiety in which the bridge between the two pyridocarbazole moieties is a polyoxyethylenyl group or an aminobispolyoxyethylenyl group.

### CROSS-REFERENCE TO RELATED APPLICATIONS

The subject patent application claims the benefit of priority to U.S. Provisional Patent Application No. 63/094,117 (filed Oct. 20, 2020; now pending). The full disclosure of the priority application is incorporated herein by reference in its entirety and for all purposes.

### STATEMENT OF GOVERNMENT SUPPORT

This invention was made with government support under grant numbers NS096898, NS099114, NS116846 and NS097273 awarded by the National Institute of Health. The government has certain rights in the invention.

### BACKGROUND

Amyotrophic lateral sclerosis (ALS) and frontotemporal dementia (FTD) are progressive neurodegenerative disorders that manifest by motor impairment and cognitive, behavioral, and language deficits. The most common genetic cause of ALS and FTD is a microsatellite sequence, the GGGGCC hexanucleotide repeat expansion [G₄C₂^{exp}] in intron 1 of chromosome 9 open reading frame 72 (C9orf72), and the associated disease has been designated c9ALS/FTD (1, 2). While healthy individuals typically carry 2-30 G₄C₂ repeats, individuals with C9orf72-associated ALS or FTD (c9ALS/FTD) typically harbor hundreds to thousands of repeats (3, 4). Various studies have shown that the RNA transcribed from the expanded GGGGCC repeat, r(G₄C₂)^{exp}, is a toxic agent that plays a role in the development of c9ALS/FTD pathogenesis (FIG. 1A). Indeed, r(G₄C₂)^{exp}: (i) can operate by a gain-of-function mechanism in which the repeat binds and sequesters a subset of proteins affecting gene expression (1); (ii) is aberrantly translated into toxic dipeptide repeat proteins (DPRs) via repeat associated non-ATG (RAN) translation (5-9); and (iii) results in the formation of r(G₄C₂)^{exp}-containing foci in a subset of CNS cells (9, 10).¹ Previous studies have shown that r(G₄C₂)^{exp}-associated toxicity can be mitigated by small molecules that selectively bind r(G₄C₂)^{exp} (FIG. 1B) (12, 13). ¹ The C9orf72 antisense strand is also transcribed, producing r(G₂C4)^{exp}, which is also RAN translated and forms foci (8, 9, 11).

An objective, therefore, is to advance earlier studies and develop a treatment for ALS/FTD that displays potent ability to ameliorate the deleterious pathogenesis of the RNA transcribed from the expanded repeat and to achieve a significant therapeutic index.

### SUMMARY OF THE INVENTION

The present invention is directed to methods for treatment of a microsatellite originated disease, ALS/FTD. Aspects of the method relate to embodiments of an ALS compound which is capable of binding and/or complexing with the RNA sequence transcribed from the microsatellite G₄C₂ repeat in the C9ORF72 genotype. Embodiments of the ALS compound comprise in part a polycyclic heteroaromatic compound, specifically a pyridocarbazole moiety. The embodiments further comprise at least a substituent bound to the pyridocarbazole moiety comprising hydroxy, alkoxy or a bridging group bound to two pyridocarbazole moieties to form at least a dimer embodiment. In particular, embodiments of the ALS compound comprise a pyridocarbazole moiety with X according to Formula I

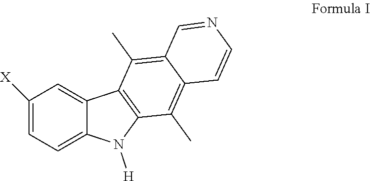

wherein X is hydrogen, hydroxyl, C₁ to C₄ alkoxy or a bridging group comprising a bridging polyoxyethylenyl group or a bridging R-aminobispolyoxyethylenyl group wherein the bridging group has the pyridocarbazole moiety covalently attached to each of its termini. The R-amino group of the R-aminobispolyoxyethylenyl group is R—N with R as hydrogen, acetyl, an RNase recruiting moiety or a 4,4′-diaza-oct-7-yn-1-oyl group. With X as a bridging group, the resulting ALS compound comprises a bridged dimer of the pyridocarbazole moiety, also described herein as a polyoxyethylenyl bispyridocarbazole and an optionally N-substituted aminobispolyoxyethylenyl bispyridocarbazole. Embodiments of the ALS compound also comprise the pharmaceutically acceptable salts thereof.

The bridged dimer or bis bridged pyridocarbazole may comprise Formula II

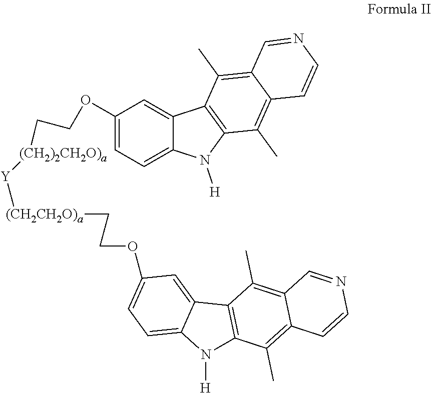

wherein a is an integer of 1 to 5, preferably 2 to 5, more preferably 2 or 2, especially more preferably 2, Y is oxygen or R—N wherein R is hydrogen, acetyl, the RNase recruiting moiety bound to N comprising Formula III or the diaza-4,4′-oct-7-yn-1-oyl group bound to N comprising Formula IV or a succinoyl group comprising Formula V:

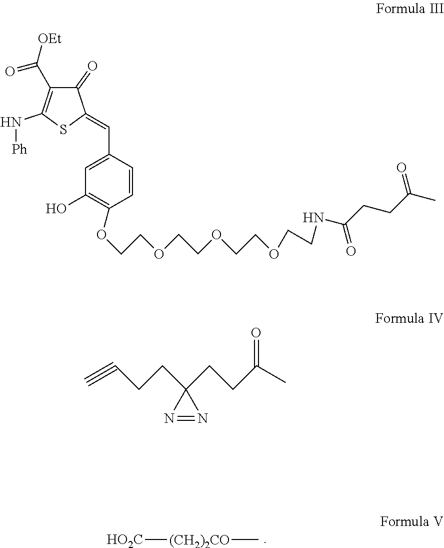

Formula V provides a link for bonding an amine such as an RNase recruiting moiety to the bridging Y of Formula II when Y is NH.

Preferable embodiments of the dimer include those in which Y is R—N. Especially preferable of these embodiments are those in which a is an integer of 2 or 3, more preferably 2. The more preferable embodiment of R is Formula III. Most preferable of these embodiments is one in which a is 2 and R is Formula III.

Compositional embodiments of the invention include the bis bridged pyridocarbazole of Formula II and the preferred compositional embodiment Formula II wherein Y is Formula III or IV. The pharmaceutically acceptable salts of these compositional embodiments are also included as aspects of the invention. In addition, a compositional embodiment is included which comprises Formula II with Y as an isomer of Formula III. The Formula III isomer has the meta attachment of the polyoxyethylenyl group to the catchol group (dihydroxybenzene group) instead of the para attachment shown by Formula III. This Formula III isomer is inactive as an L-RNase recruiting moiety.

Additional embodiments include methods for complexing and/or binding the ALS compound with the RNA repeat r(G₄C₂)^{exp} which is r(G₄C₂)ₘ with m as a integer designator of 1-1,000, preferable 2 to 500, more preferably 2 to 200, most preferable 2 to 30. These embodiments include methods for complexing and/or binding an abnormal number or RNA repeats in which m is at least 100, preferably at least 200, and more preferably at least 500 to at least 1000. For these embodiments the RNA repeat sequence is at least an RNA hairpin structure.

Further embodiments include methods in which the RNA repeat is present in cells such as but not limited to cell cultures, HEK293T cells transfected to express the RNA repeat expansion, ALS patient-derived cells, lymphoblastoid cells, induced pluripotent stem cells (c9 iPSCs cells) and iPSC-derived spinal neuron cells (c9 iPSNs). The RNA repeat may also be present as c9ALS/FTD BAC cells of a transgenic mouse. The RNA repeat r(G₄C₂)^{exp} may be present or may be transcribed in these cells when the cells contain chromosome 9 open reading frame 72 known as C9orf72 and r(G₄C₂)^{exp} as an abnormal repeat present in intron 1 of C9orf72.

Embodiments of the methods also enable the ALS compound as Formula II with Formula III to decrease and/or inhibit RAN translation of r(G₄C₂)^{e}XP RNA in cells including but not limited to those mentioned above. Further these methods preferably do not inhibit transcription of the C9orf 72.

Embodiments according to the invention further include pharmaceutical compositions comprising an ALS compound and a pharmaceutically acceptable carrier. Preferably, the ALS compound comprises Formula II with Formula III. More preferably, the ALS compound of Formula II/III has designation a as 2. Preferably, the pharmaceutical composition comprises an effective amount, preferably an effective dose of the ALS compound for treatment of ALS/FTD disease.

Embodiments according to the invention also include a method of treatment of patients suffering from ALS/FTD. These embodiments comprise administration of an effective amount of an ALS compound of Formula II/III, preferably with designation a as 2. These embodiments also comprise administration of a pharmaceutically acceptable composition with an effective amount or effective dose of the ALS compound of Formula II/III. Preferably routes of administration include oral, intraperitoneal (ip), intravenous (iv), intramuscular (im), subcutaneous (SC), oral, rectal, vaginal, intrathecal, and/or intradermal. Preferably, the disease is amyotrophic lateral sclerosis.

Additional embodiments according to the invention include methods for treatment of other diseases caused by r(G₄C₂) RNA repeat expansions. While ALS and FTD are two extremes of the disease spectrum associated with this repeat expansion, the disease spectrum includes a range of neuropsychological deficits such as cognitive impairment, behavioral impairment, and several other manifestations. All of these diseases may be treated as described herein for treatment of ALS/FTD and the ALS-FTD disease spectrum. See for example, M. B. Leko, et. al., Behav, Neurol., v. 2019, Jan. 15, 2019, 2909168.

### DESCRIPTION OF DRAWINGS

FIG. 1 depicts small molecules targeting r(G₄C₂)^{exp} in C9orf72 selectively affect disease-associated pathways. (A) and (B) show proposed pathology of r(G₄C₂)^{exp} in C9orf72 for neurotoxicity. The r(G₄C₂)^{exp} can generate dipeptide repeat proteins (GA, GP and GR) by RAN translation, sequester RNA binding proteins affecting gene expression, and forms RNA foci in a subset of cells. Using our lead identification strategy, Inforna, we identified small molecules which selectively binding to the G/G loop in the RNA hairpin structure formed by r(G₄C₂)^{exp}. A dimerization strategy was applied to target the repeat sequence with optimization of the linker. (C) illustrates the monomer compound used in this application. Compound 1 was previously reported to selectively binding to the G:G loop. (D) shows how Compound 2 was obtained by linker optimization. An amino NH group was introduced into the PEG linker to generate compound 3 for higher binding affinity.

FIG. 2 depicts how Compound 3 recognizes the three-dimensional structure of r(G₄C₂)^{exp} in cells and inhibits disease-associated RAN translation in a variety of cellular models. (A) shows a 3D model of 3 bound to a r(G₄C₂) repeat model construct, as determined by NMR spectroscopy and MD simulations. MSMS representation in VMD was used to display the lowest binding energy state. Compound 3 is highlighted in cyan. (B) shows a schematic representation of the transcript variants from C9orf72 that were measured by RT-qPCR. (C) provides a schematic representation of Chem-CLIP method. (D) shows a Fold-enrichment of C9orf72 mRNA variants in Chem-CLIP studies completed in c9ALS patient-derived LCLs or iPSC. Intron 1 harbors the r(G₄C₂)^{exp} repeat expansion while transcript variants containing Exon 1b do not (n=1 C9orf72 LCL and 1 C9orf72 iPSC line, 3 replicates per line). (E) illustrates the inhibitory effect of 3 on RAN translation, as assessed in HEK293T cells dually transfected with (G₄C₂)₆₆-Nano Luciferase (disease) and SV40-Firefly (normalization) (n=3 replicates). (F) shows the poly(GP) abundance in protein lysates from patient-derived lymphoblastoid cells treated with 3. Quantification was normalized to vehicle (n=3 C9orf72 LCL lines, 3 replicates per line). (G) shows the poly(GP) abundance in protein lysates from patient-derived iPSCs treated with 3. Quantification was normalized to vehicle (n=4 C9orf72 iPSC lines, 3 replicates per line). (H) shows the percentage of (G₄C₂)₆₆-No ATG-GFP mRNA transcript present within monosome- and polysome-containing fractions from HEK 293T cells treated with vehicle or 200 nM of 3, quantified by RT-qPCR analysis of each fraction (n=2 independent experiments). Fractions from the polysome gradients labeled as “Monosomes” contain 405, 605, and 80S ribosomal subunits (fractions 1-7); “LMW” indicates low molecular weight polysomes (fractions 8-10); and “HMW” indicates high molecular weight polysomes (fractions 11-12). *, P<0.05; **, P<0.01; ***, P<0.001; and ****, P<0.0001, as determined by a Repeated Measures ANOVA test with Tukey's multiple comparisons (panels F & G), a One-way ANOVA test with multiple comparisons (panel E), or unpaired t-test with Welch's correction (panels D & H). Error bars indicate SD.

FIG. 3 illustrates how a designer small molecule binds to r(G₄C₂)^{exp} and recruits RNase L to selectively cleave r(G₄C₂)^{exp} in cells. (A) discloses the proposed functionality of an RNase L-recruiting small molecule when bound selectively to a target RNA with readout by RT-qPCR and RNA sequencing (RNA-seq). (B) shows that Compound 6 is a derivative featuring the RNA-binding modules but lacks the RNase L recruiting module and was used as a control. (C) shows that Compound 7 was obtained by conjugation of a nuclease RNase L recruiter module to a r(G₄C₂)^{exp}-targeting dimer. Control compound 8 is a dimer conjugated to an inactive RNase L-recruiting module with altered regioselectivity.

FIG. 4 discloses how RIBOTAC 7 degrades r(G₄C₂)^{exp} in a cellular model of c9ALS/FTD by local recruitment of RNase L. (A) illustrates the inhibitory effect of 7 on RAN translation, as assessed in HEK293T cells dually transfected with (G₄C₂)₆₆-No ATG-Nano-Luciferase (disease) and SV40-Firefly (normalization) (n=3 replicates). (B) shows the effect of 7 on r(G₄C₂)^{exp} levels in HEK293T cells transfected with a plasmid encoding (G₄C₂)₆₆-No ATG-GFP (n=3 biological replicates) and ablation of this effect upon RNase L knockdown with an RNase L-targeting siRNA (n=3 replicates). (C) shows knockdown of RNase L levels by siRNA in HEK293T cells (n=3 replicates). (D) shows a scheme of an RNase L immunoprecipitation (IP) assay to study ternary complex formation between r(G₄C₂)₆₆, RNase L, and 7. (E) shows the results of the RNase L IP assay, demonstrating that a ternary complex indeed forms in cellulis (n=3 replicates). *, P<0.05; **, P<0.01; ***, P<0.001; and ****, P<0.0001, as determined by a One-way ANOVA with multiple comparisons (panels A-C), or unpaired t-test with Welch's correction (panel E). Error bars indicate SD.

FIG. 5 shows how the r(G₄C₂)^{exp}-targeting RIBOTAC 7 selectively cleaves the r(G₄C₂)^{exp}-containing intron 1 in C9orf72 and alleviates disease associated defects in patient-derived cells. (A) shows the effect of 7 on C9orf72 intron 1 levels, which harbors r(G₄C₂)^{exp}, in patient-derived lymphoblastoid cells, as determined by RT-qPCR with intron 1-specific primers (n=3 C9orf72 LCL lines, 3 replicates per line). (B) shows the effect of co-treating patient-derived lymphoblastoid cells with parent dimer 6 (increasing concentrations) and RIBOTAC 7 (constant concentration), as determined by RT-qPCR using intron 1-specific primers (n=1 C9orf72 LCL line, 3 replicates per line). (C) shows the effect of 7 and 8 in lymphoblastoid cells from healthy donors that do not express the RNA repeat expansion, as determined by quantification of intron 1 levels by RT-qPCR (n=1 control LCL line, 3 replicates per line). (D) shows the effect of 7 on Poly(GP) abundance in protein lysate extracted from patient-derived lymphoblastoid cells (n=3 C9orf72 LCL lines, 3 replicates per line). (E) shows the effect of 7 on C9orf72 exon 1b levels, present only in transcripts that do not have r(G₄C₂)^{exp}, in patient-derived lymphoblastoid cells, as determined by RT-qPCR with exon 1b-specific primers (n=1 C9orf72 LCL line, 3 replicates per line). (F) shows the effect of 7 (500 nM) or an ASO complementary to the repeat (100 nM) on human transcripts containing short, non-pathogenic r(G₄C₂) repeats in a patient-derived LCL (n=1 C9orf72 LCL line, 3 replicates per line), as determined by RT-qPCR using gene-specific primers (Table 2) and compared to vehicle. The relative abundance of each target RNA was calculated relative to GAPDH. The sequence of the ASO is complementary to the repeat expansion itself [5′-mG*mG*mC*C*C*C*G*G*C*C*C*C*G*G*C*C*C*mC*mG*mG, where m indicates a 2′-O-methyl residue and * indicates a locked nucleic acid (LNA) residue]. This ASO is different from a previously reported ASO that targets the intron upstream of the repeat (11, 23-25). (G) shows the effect of 7 on C9orf72 intron 1 levels, which harbors r(G₄C₂)^{exp}, in patient-derived iPSCs, as determined by RT-qPCR with intron 1-specific primers (n=4 C9orf72 iPSC lines, 3 replicates per line). (H) shows the effect of 7 on Poly(GP) abundance in protein lysate extracted from patient-derived iPSCs (n=4 C9orf72 iPSC lines, 3 replicates per line). (I) shows the effect of 7 on C9orf72 intron 1 levels, which harbors r(G₄C₂)^{exp}, in patient-derived iPSNs, as determined by RT-qPCR with intron 1-specific primers (n=2 C9orf72 iPSC lines, 3 replicates per line). (J) shows the effect of 7 on Poly(GP) abundance in protein lysate extracted from patient-derived iPSN (n=1 C9orf72 iPSC line, 3 replicates per line). (K) illustrates Intron 1: Exon 2 as determined by RNA-seq analysis of patient-derived iPSNs treated with 7 relative to vehicle (n=1 C9orf72 iPSC line, 3 replicates per line). *, P<0.05; **, P<0.01; ***, P<0.001; and ****, P<0.0001, as determined by a Repeated Measures ANOVA test with Tukey's multiple comparisons (panels A, D, & G-I), a One-way ANOVA test with multiple comparisons (panels B-C, E-F, & J), or unpaired t-test with Welch's correction (panel K). Error bars indicate SD.

FIG. 6 shows how the r(G₄C₂)^{exp}-targeting RIBOTAC 7 alleviates c9ALS-FTD-associated toxicity in a pre-clinical mouse mode of disease. (A) shows the effect of 33 nmol of 7 on r(G₄C₂)^{exp}-containing intron 1 levels in the +/+PWR500 C9orf72 BAC mouse model (Liu et al., 2016), as determined by RT-qPCR analysis of total RNA isolated from treated and untreated mice with intron 1-specific primers (n=6; each data point represents a sample from a different mouse). (B) shows the effect of 7 on C9orf72 exon 1b levels, found only in transcripts lacking r(G₄C₂)^{exp}, in +/+PWR500 mice, as determined by RT-qPCR analysis of total RNA isolated from treated and untreated mice with exon 1b-specific primers (n=6; each data point represents a sample from a different mouse). (C) shows the effect of 7 on Poly(GP) abundance in brain tissue harvested from +/+PWR500 mice, as determined by a sandwich immunoassay. Relative Poly(GP) abundance was normalized to total protein levels and vehicle treated mice (n=6; each data point represents a sample from a different mouse). (D) shows the effect of 7 on the abundance of control protein β-actin abundance in brain tissue harvested from +/+PWR500 mice, as determined by a sandwich immunoassay. Relative β-actin abundance was normalized to total protein levels and vehicle treated mice (n=6; each data point represents a sample from a different mouse). Arrows point to r(G₄C₂)^{exp} foci. (E) shows the effect of 7 on r(G₄C₂)^{exp}-containing foci in +/+PWR500, as determined by RNA-FISH (n=3). (F) shows representative histological images of cortex from PWR500+/+ mice treated with 7 compared with vehicle, visualizing Poly(GP), Poly(GA), or TDP-43. Scale bars 200 μm. Arrows point to Left: Poly(GP) aggregates, Middle: Poly(GA) aggregates, Right: TDP-43 inclusions. (G) shows quantification of Poly(GP) aggregates from histological analysis, including images in F (n=4 mice; all cells (˜200) were counted per section; 3 sections counted per mouse). (H) shows quantification of Poly(GA) aggregates from histological analysis, including images in F (n=4 mice; all cells (˜200) were counted per section; 3 sections counted per mouse). (I) shows quantification of TDP-43 inclusions from histological analysis, including images in F (n=4 mice; all cells (˜200) were counted per section; 3 sections counted per mouse). *, P<0.05; **, P<0.01; ***, P<0.001, ***, *P<0.0001, as determined by an unpaired t-test with Welch's correction. Error bars indicate SD.

FIG. 7 shows small molecules targeting r(G₄C₂)^{exp} directly engage its structure as determined by affinity measurements, NMR spectroscopy and MD simulations, and Chemical Cross-Linking and Isolation by Pull-down (Chem-CLIP) in vitro and in cells:

(A) shows dissociation constants of 2 and 3 for binding to r(G₄C₂)₈, d(G₄C₂)₈, base pair control r(GGCC)₈, and antisense r(G₂C₄)₈ obtained via BLI. Data are reported as means SD (n=2 independent experiments). (B) shows the 1D NMR spectrum of 3 bound to r(G₄C₂)₄. (C) shows the 1D NMR spectrum of 3 and a fully paired construct, r(GGCC)₄, in which no binding was observed. (D) shows the 2D NMR spectrum of 3 bound to r(G₄C₂)₄. (E) shows the structures of Chem-CLIP probes 4 and control Chem-CLIP probe 5, which lacks the RNA-binding modules. (F) shows a schematic of Chem-CLIP. (G) shows Chem-CLIP studies of 4 and r(G₄C₂)₈ in vitro. Left, plot of percentage of r(G₄C₂)₈ pulled down as a function of 4 or 5 concentration (n=3 independent experiments). Middle, plot of percentage of r(G₄C₂)₈ pulled down by 4, as a function of the concentration of 3, or a Competitive (C-)Chem-CLIP experiment (n=3 independent experiments). Right, plot of percentage of r(G₄C₂)₈, r(G₄C₂)₈+K+(quadruplex form), r(G₂C₂)₁₀, d(G₄C₂)₈, d(G₂C₂)₁₀ pulled down by 4 (n=3 independent experiments). (H) shows an ASO-Bind-Map target profiling of 3 in c9ALS patient-derived lymphoblastoid cells (n=3 biological replicates). *P, <0.05; **, P<0.01; **, *P<0.001; and ****, P<0.0001, as determined by a One-way ANOVA test with multiple comparison (panel G & H). Error bars indicate SD.

FIG. 8 shows how Compound 3 selectively affects various c9ALS/FTD-associated defects in various cellular models of disease. (A) shows relative levels of r(G₄C₂)^{exp} upon 3-treatment of transfected HEK293T cells (derived from r(G₄C₂)66-No ATG-GFP), patient-derived lymphoblastoid cells (intron 1 levels), and patient-derived iPSCs (intron 1 levels), as quantified by RT-qPCR (n=1 C9orf72 LCL and C9orf72 iPSC line, 3 replicates per line). (B) shows a representative gel image of the inhibition of topoisomerase II activity by 3 and 7, quantified as percentage linearized DNA compared to the positive control compound VP-16. (C) shows the relative abundance of WT C9ORF72 protein (bottom band) in patient-derived iPSC cells treated with vehicle or 3 at 5, 50, 500 nM, as determined by Western blotting and normalized to β-actin (n=1 C9orf72 iPSC line, 3 replicates per line). (D) shows a cell viability assay in patient-derived lymphoblastoid cells treated with 3 at a range of concentrations, as measured by CellTiter-Glo®. Viability was normalized to the DMSO treated samples (n=3). (E) shows the effect of 3 on total C9orf72 mRNA levels, as determined by RT-qPCR using primers for Exon 2-Exon 3 junction present in all transcripts in c9ALS patient-derived (n=3). Error bars indicate SD.

FIG. 9 shows how Compound 3 reduces the number of RNA foci in patient-derived lymphoblastoid cells. (A) shows representative images of the effect of 3 on r(G₄C₂)^{exp}-containing nuclear foci in patient-derived lymphoblastoid cells. (B) shows quantification of number of foci per nucleus in a single line of patient-derived LCLs treated with vehicle or 100 [nM] of 3 (n=1 C9orf72 LCL, 3 replicates; 200 nuclei counted per biological sample). Scale bars are 20 μm. ***P<0.001, as determined by an unpaired t-test with Welch's correction. Error bars indicate SD.

FIG. 10 shows how Compound 3 functions by inhibiting ribosome loading. (A) shows polysome profiles generated from HEK293T cells transfected with (G4C2)66-NoATG-GFP upon treatment with vehicle or 3 (200 nM). Polysome fractionation profiles are representative of two independent experiments. (B) shows the percentage of (G4C2)66-No ATG-GFP mRNA and GAPDH mRNA present in each fraction treated with vehicle or 3 (n=2). **, P<0.01; ***, P<0.001; and ****, P<0.0001, as determined by a One-way ANOVA test with multiple comparison. Error bars indicate SD.

FIG. 11 shows the ribonuclease recruiting compound 7 elicits cleavage of r(G₄C₂)^{exp} in vitro. (A) shows a schematic of detection of RNase L recruitment and cleavage of r(G4C2)4 by 7 using a FRET-based assay. (B) shows the percent cleavage of r(G4C2)4 by recruitment of RNase L (25 nM) as a function of 7 concentration (n=2 independent measurements). Background cleavage was subtracted out using signal from vehicle-treated samples. (C) shows the cleavage of r(G4C2)4 by RNase L recruitment by 7, as determined using 5′-[g-32P] RNA and gel electrophoresis. Left, Representative autogradiogram. Right, Quantification of gel autogradiograms. Relative r(G4C2)4 cleavage was calculated relative to vehicle-treated samples (n=2 independent measurements). *, P<0.05; **, P<0.01; ***, P<0.001; and ****, P<0.0001, as determined by a One-way ANOVA test with multiple comparison. Error bars indicate SD.

FIG. 12 shows how the RIBOTAC compound 7 alleviates various c9ALS/FTD defects in patient-derived cells. (A) shows the effect of a repeat-targeting ASO (100 nM) on C9orf72 intron 1 levels, which harbors r(G4C2)exp, treated in all cell lines tested (n=1 C9orf72 LCL and 1 C9orf72 iPSC line, 3 replicates per line). The sequence of the ASO is complementary to the repeat, 5′-mG*mG*mC*C*C*C*G*G*C*C*C*C*G*G*C*C*C*mC*mG*mG, where m indicates a 2′-O-methyl residue and * indicates a locked nucleic acid (LNA) residue. This ASO is different from a previously reported ASO that targets the intron upstream of the repeat (1-4). Vehicle indicates mock transfected cells. (B) shows the effect of a repeat-targeting ASO (100 nM) on Poly(GP) abundance treated in all cell lines tested (n=1 C9orf72 LCL and 1 C9orf72 iPSC line, 3 replicates per line). ASO as described in A. Vehicle indicates mock transfected cells. (C) shows the effect of 8, the control, inactive RIBOTAC that cannot recruit RNase L, in LCLs from c9ALS donors that express the RNA repeat expansion, as determined by quantification of intron 1 levels by RT-qPCR (n=1 C9orf72 LCL line, 3 replicates per line). (D) shows the effect of 7 on native C9ORF72 protein levels in c9ALS patient-derived iPSCs, as determined by Western blotting. Left, Representative image of a Western blot; Right, Quantification of native C9ORF72 protein levels, as normalized to levels of 3-actin (n=1 C9orf72 iPSC line, 3 replicates per line). (E) shows an RNA-seq analysis of patient-derived iPSNs treated with 50 nM of 7 (n=1 C9orf72 iPSC line, 3 replicates per line). The amounts of mutant allele containing the repeat expansion were calculated using the coding SNPs as a proxy for abundance. All other genes were plotted as standard in the field. **, P<0.01; **, P<0.001; and ****, P<0.0001, as determined by a One-way ANOVA test with multiple comparison (panels C & D), or unpaired t-test with Welch's correction (panels A & B). Error bars indicate SD.

FIG. 13 shows that RIBOTAC 7 has no effect in iPSCs from healthy donors. (A) shows the effect of 7 on C9orf72 intron 1 abundance, which harbors r(G₄C₂)^{exp}, in iPSC lines from healthy donors, as determined by RT-qPCR with intron 1-specific primers (n=4 control iPSC lines, 3 replicates per line). (B) shows the effect of 7 on C9orf72 intron 1 abundance, which harbors r(G₄C₂)^{exp}, in iPSCs from healthy donors, as determined by RT-qPCR with intron 1-specific primers relative to GAPDH, 18S, and b-actin housekeeping genes (n=3 for each cell line). (C) shows the effect of 7 on C9orf72 exon 2-3 abundance in iPSCs from healthy donors, as determined by RT-qPCR with exon 2-3-specific primers relative to GAPDH, 18S, and b-actin housekeeping genes (n=3 for each cell line). Error bars indicate SD.

FIG. 14 shows how RIBOTAC 7 alleviates C9orf72 hallmarks across multiple patient-derived cell lines with enhanced potency. (A) shows the effect of 7 on C9orf72 intron 1 levels, which harbors r(G4C2)^{exp}, in iPSC lines from healthy donors, as determined by RT-qPCR with intron 1-specific primers (n=4 control iPSC lines, 3 replicates per line). (B) shows a time-course evaluation on the return of Poly(GP) abundance after treatment with 50 nM of 3 or 7. Poly(GP) levels were measured at the indicated time post compound withdrawal (n=1 C9orf72 iPSC line, 3 replicates per line). (C) shows a direct comparison between 3 and 7 on reduction of Poly(GP) abundance in protein lysate extracted from c9ALS patient-derived iPSCs (n=4 C9orf72 iPSC lines, 3 replicates per line). (D) shows the effect of 7 on C9orf72 intron 1 levels, which harbors r(G₄C₂)^{exp}, in iPSNs from healthy donors, as determined by RT-qPCR with intron 1-specific primers (n=2 control iPSC lines, 3 replicates per line). (E) shows the effect of 50 nM of 7 on Poly(GP) abundance in protein lysate extracted from an additional patient-derived iPSN cell line (n=1 C9orf72 iPSC line, 3 replicates per line). *, P<0.05; **, P<0.01; and ***, P<0.001, as determined by a Repeated Measures ANOVA with Tukey's multiple comparison (panels A, C, & D), a One-way ANOVA with multiple comparison (panel B), or unpaired t-test with Welch's correction (panel E). Error bars indicate SD.

FIG. 15 shows how RIBOTAC 7 alleviates c9ALS/FTD-associated pathology in a mouse model. (A) shows the effect of 33 nmol of 7 on total C9orf72 mRNA levels upon treatment of +/+PWR500 mice, as determined by RT-qPCR using primers for Exon 2-Exon 3 junction present in all transcripts. Note, C9orf72 mRNA levels are reduced by the same levels accounting for intron 1 abundance, indicating selectivity for the repeat expansion (n=6 mice per treatment group). (B) shows an analysis of neurons from treated and untreated WT mice by microscopy, including RNA FISH [showing absence of r(G4C2)exp]. Scale bars 200 μm (n=3 mice per treatment group). (C) shows the effect of 7 on β-actin protein levels in +/+PWR500 mice (n=6 mice per treatment group). (D) shows an analysis of brain histology of treated and untreated WT mice including Poly(GP) and Poly(GA) staining. Scale bars 200 μm. (E) shows RNA-seq analysis of +/+PWR500 mice treated with 33 nmol of 7 (n=3 mice per treatment group). The amounts of mutant allele containing the repeat expansion were calculated using the coding SNPs as a proxy for abundance. All other genes were plotted as standard in the field. *P, <0.05; **, P<0.01; ***, P<0.001; and ***, P<0.0001, as determined by an unpaired t test with Welch's correction. Error bars indicate SD.

FIG. 16 shows that RIBOTAC 7 selectively reduces abundance of the C9orf72 repeat-containing intron in c9ALS patient-derived cells by RNA sequencing. (A) shows RNA-seq analysis of c9ALS patient-derived iPSCs treated with 50 nM of 7 plotted as average Log 2(Fold Change) vs −Log 10(q-value) (n=1 C9orf72 iPSC line, 3 replicates per line). The amounts of mutant allele containing the repeat expansion were calculated using the coding SNPs as a proxy for abundance. All other genes were plotted as standard in the field. (B) shows a number of sequencing reads of C9orf72 in c9ALS patient-derived iPSCs treated with 50 nM of 7 normalized to vehicle treated cells (n=1 C9orf72 iPSC line, 3 replicates per line). (C) shows the ratio of Intron 1:Exon 2 as determined by RNA-seq analysis of c9ALS patient-derived iPSCs treated with 7 relative to vehicle (n=1 C9orf72 iPSC line, 3 replicates per line). (D) shows an RNA-seq analysis of iPSCs from healthy donors treated with 50 nM of 7 plotted as average Log 2(Fold Change) vs −Log 10(q-value) (n=1 C9orf72 iPSC line, 3 replicates per line). The amounts of mutant allele containing the repeat expansion were calculated using the coding SNPs as a proxy for abundance. All other genes were plotted as standard in the field. (E) shows a number of sequencing reads of C9orf72 in iPSCs from healthy donors treated with 50 nM of 7 normalized to vehicle treated cells (n=1 C9orf72 iPSC line, 3 replicates per line). (F) shows a ratio of Intron 1:Exon 2 as determined by RNA-seq analysis of iPSCs from healthy donors treated with 7 relative to vehicle (n=1 C9orf72 iPSC line, 3 replicates per line). (G) shows the effect of 7 on C9orf72 intron 1 abundance, which harbors r(G4C2)exp, in iPSNs from healthy donors, as determined by RT-qPCR with intron 1-specific primers (n=2 control iPSC lines, 3 replicates per line). (H) shows an RNA-seq analysis of c9ALS patient-derived iPSNs treated with 50 nM of 7 plotted as average Log 10(TPM) (n=1 C9orf72 iPSN line, 3 replicates per line). The amounts of mutant allele containing the repeat expansion were calculated using the coding SNPs as a proxy for abundance. All other genes were plotted as standard in the field. *, P<0.05, as determined by an unpaired t-test with Welch's correction (Panels B & C). Error bars indicate SD.

FIG. 17 shows that RIBOTAC 7 selectively rescues Nup98 abundance in c9ALS iPSN cells. (A) shows maximum intensity projections from SIM imaging of Nup98 in nuclei isolated from healthy and C9orf72 iPSNs following 2 weeks of treatment with ASO or 50 nM of 7. Genotype is indicated on the left while treatment is indicated on the top. (B) shows quantification of Nup98 signal. (n=2 healthy and 2 C9orf72 iPSC lines, 50 NeuN+ nuclei analyzed per line/treatment). Scale bar=5 m. ****, P<0.0001, as determined by a Two-way ANOVA with Tukey's multiple comparison.

### DETAILED DESCRIPTION

### Overview of Target and ALS Compounds

Elimination of r(G₄C₂)^{exp} could possibly ameliorate all c9ALS/FTD molecular defects, a significant advantage over targeting a particular c9ALS/FTD pathway. This strategy could warrant benefits especially with r(G₄C₂)^{exp} presence within an intron, and not an open reading frame.

To enable such an approach, a high affinity compound, a pyridocarbazole compound of Formula I with X as hydrogen was discovered and optimized (X as methoxy) to selectively recognize r(G₄C₂)^{exp} through the use of structure-activity relationships (SAR), biophysical, and structural analyses. The optimal compound, as determined through a battery of cellular assays, was then converted into a RIBOTAC by appending it to an RNase L recruiter (14). By cleaving r(G₄C₂)^{exp}, this c9ALS/FTD RIBOTAC compound (the ALS compound of Formulas II and III) ameliorates C9orf72 hexanucleotide repeat expansion (HRE)-associated pathologies in cellular model systems, including patient-derived lymphoblastoid cell lines (LCLs), patient-derived induced pluripotent stem cells (c9 iPSCs), iPSC-derived spinal neurons (c9 iPSNs), and a c9ALS/FTD BAC transgenic mouse model. Importantly, transcriptome-wide studies in iPSNs and in the transgenic mouse confirm that the RIBOTAC is selective for the expanded r(G₄C₂) repeat in intron 1 of C9orf72. These studies demonstrate that the ALS compound of Formulas II and III would provide advantageous treatment of ALS/FTD disease.

Rational Design of a Dimeric ALS Compound that Binds r(G₄C₂)^{exp}

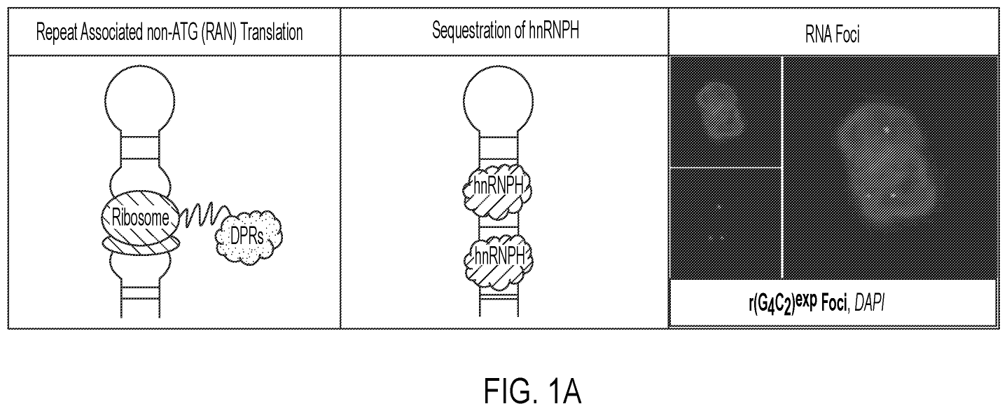

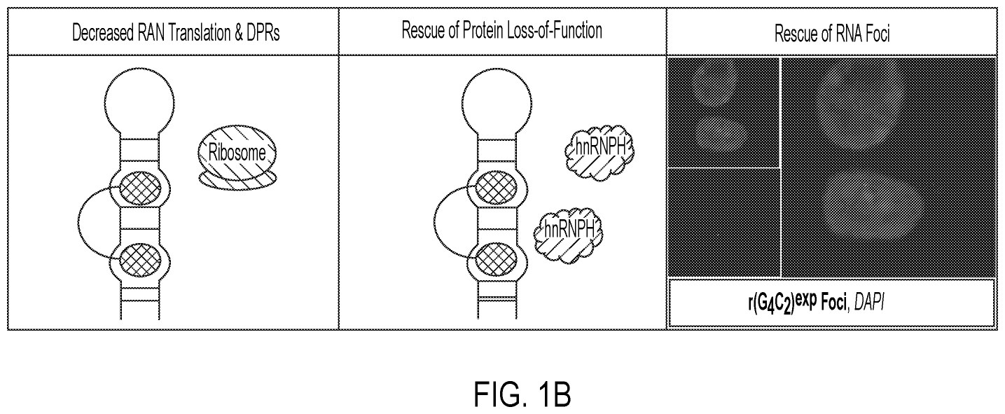

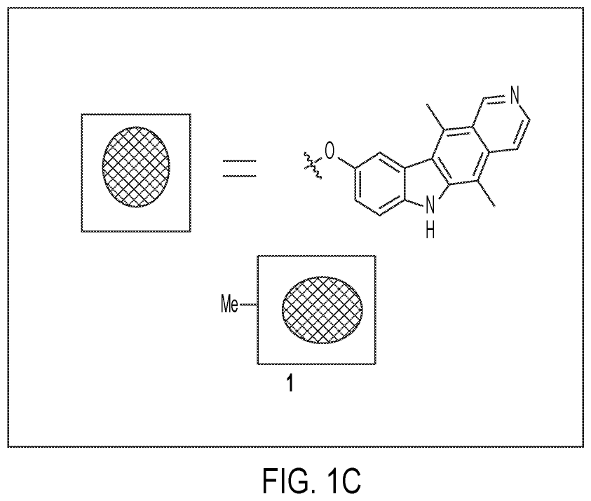

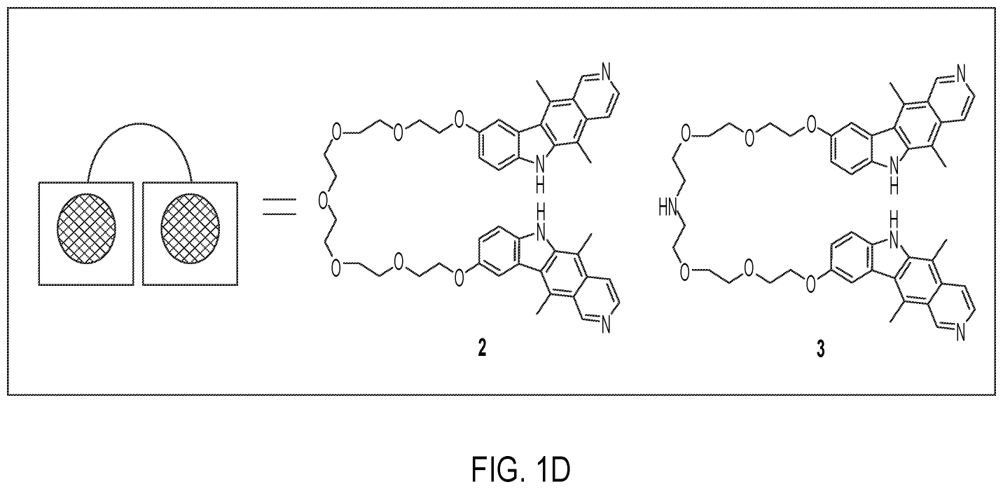

The r(G₄C₂)^{exp} that causes toxicity in c9ALS/FTD folds into two structures that are in equilibrium: a G-quadruplex and a hairpin with a periodic array of 1×1 nucleotide G/G internal loops (5′CCG̅GGG̅CC/3′GGG̅CCG̅GG) (12, 15). Using the lead identification strategy, Inforna (16, 17), and subsequent optimization, a small molecule was designed, 1 (FIG. 1-C, ALS compound of Formula I with X as methoxy), that binds these G/G internal loops selectively (12). This chemical probe enabled studies that demonstrated that the hairpin structure, not the G-quadruplex structure, is RAN translated in vitro and in cellulis. Therefore, further development of a possible treatment of ALS/FTD could involve a strategy directed to this hairpin structure.

This strategy was applied to r(G₄C₂)^{exp} (RNA repeat) guided by a model of 1 bound to r(G₄C₂) repeats (12). Positions 6 and 9 of 1 (the X position (methoxy) and the nitrogen position of the carbazole tricyclic ring of Formula I respectively) are possible sites for modification without loss of activity toward the RNA repeat. Functionalization of position 9 with an alkyne (1a) was previously synthesized (compound i7 of reference 12). Modification of position 6 with an alkyne moiety afforded 1b, (Experimental Section below). To confirm that these modifications do not affect molecular recognition, the affinities of 1a and 1b for r(G₄C₂) repeats were measured by biolayer interferometry (BLI) and compared to the affinity of 1 (ALS compound. Formula I with X as methoxy) (Table 1). Whereas the avidity of 1a (K_{d}=0.6±0.03 μM) was similar to that of 1 (K_{d}=0.3±0.03 μM), whereas 1b's avidity was ˜37-fold weaker with a K_{d} of 11±0.14 μM (Table 1). Thus, it was discovered that position 9 of the ALS compound of Formula I (X as hydroxyl) can be functionalized without affecting molecular recognition, in contrast to position 6 (nitrogen of the carbazole tricyclic ring).

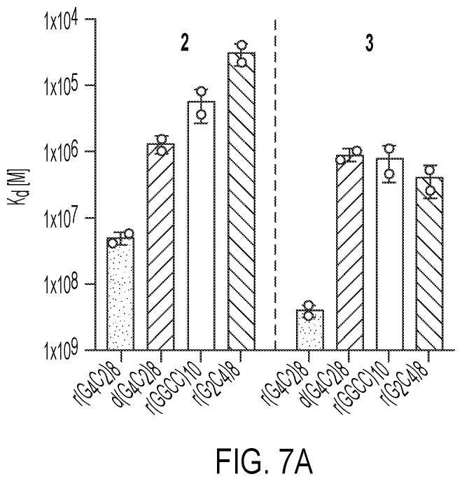

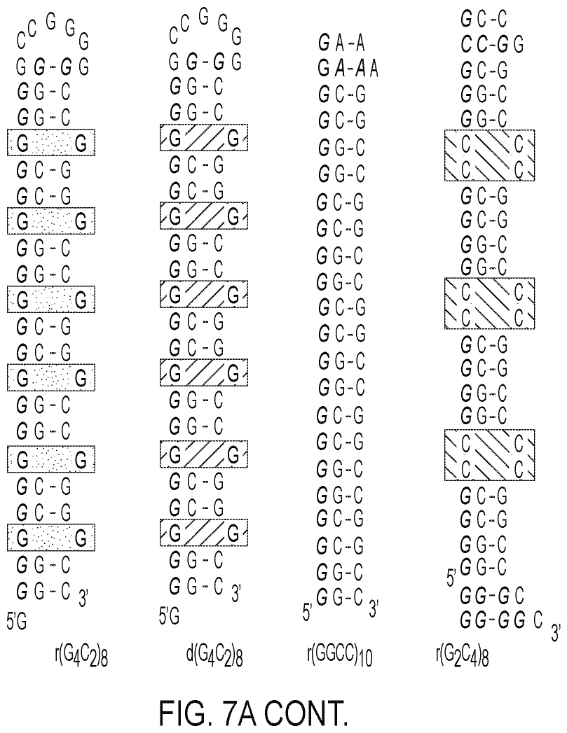

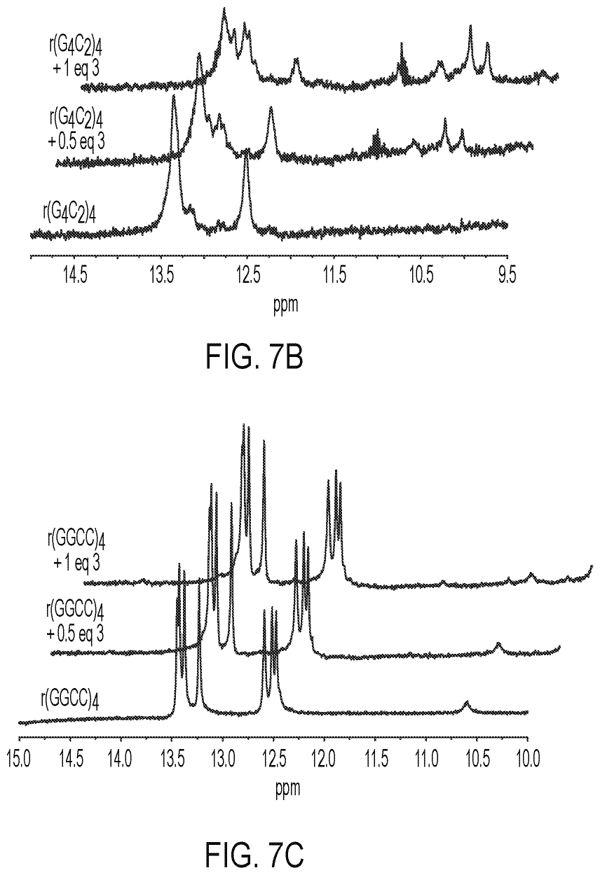

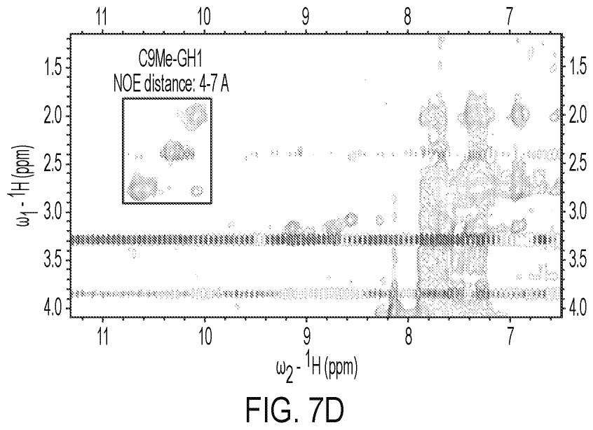

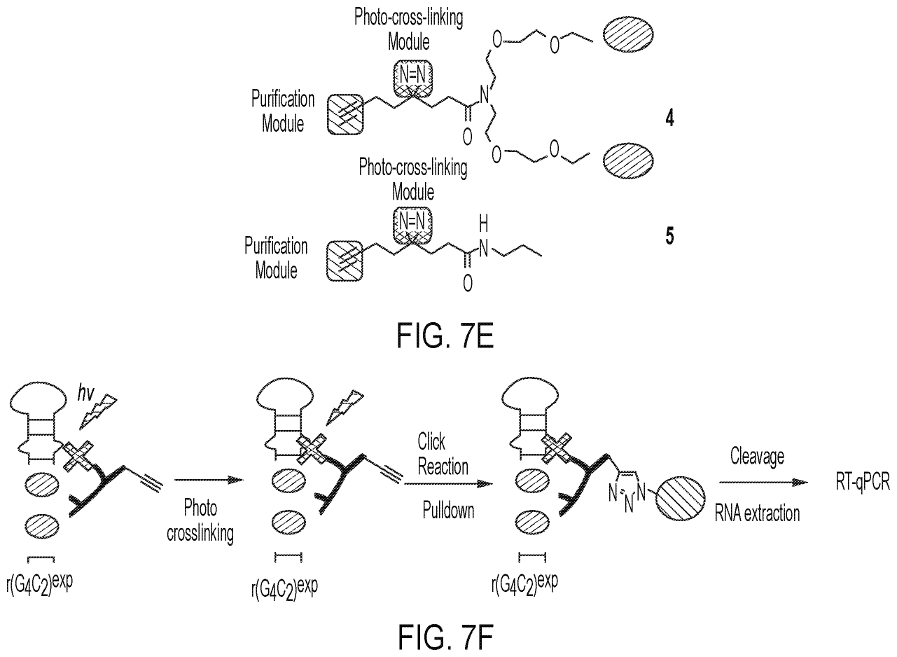

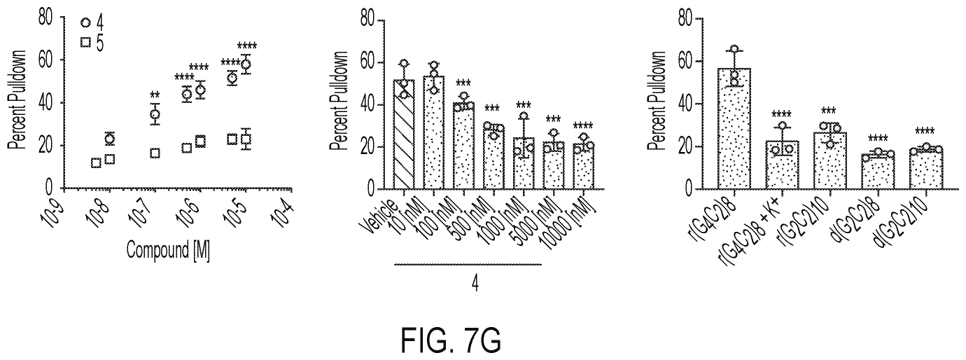

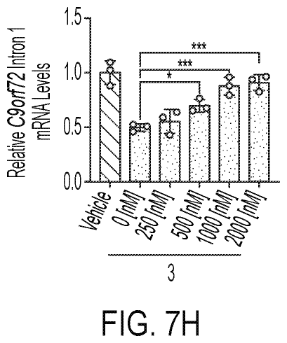

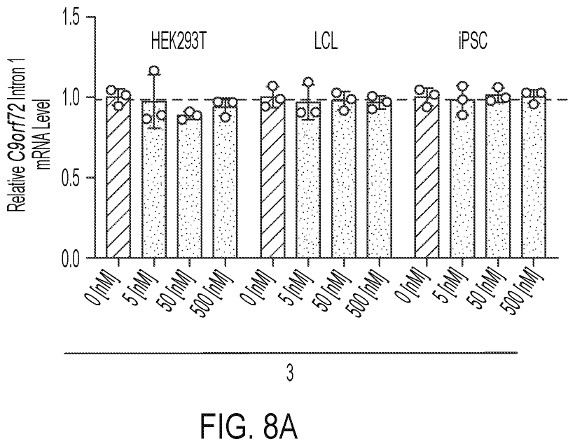

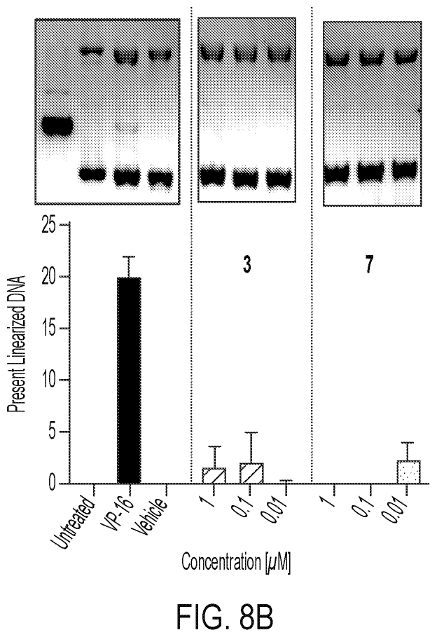

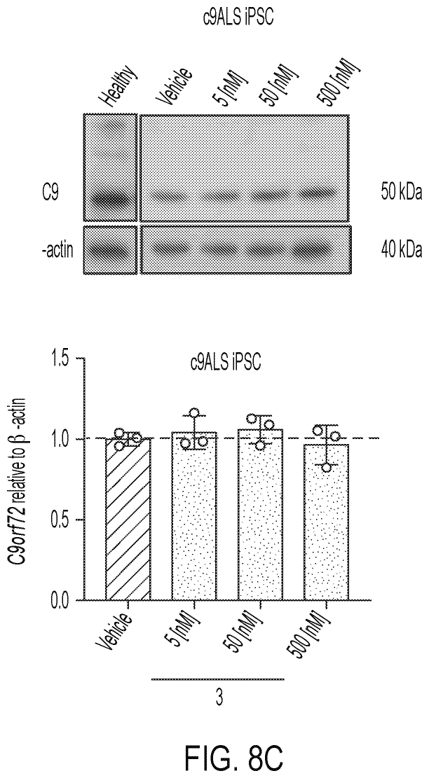

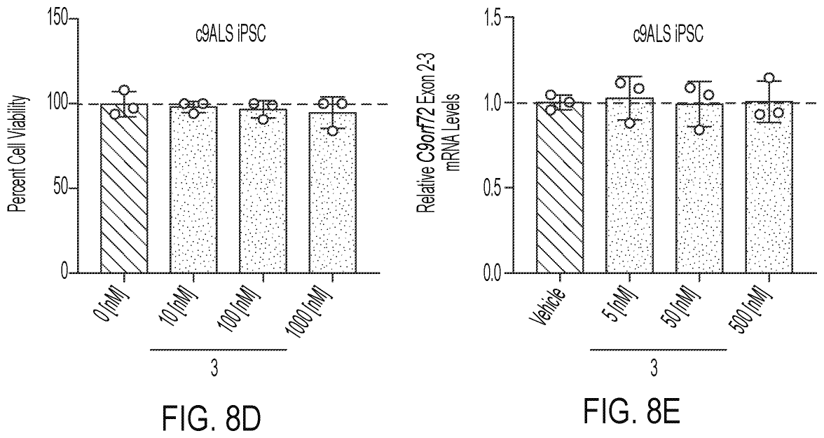

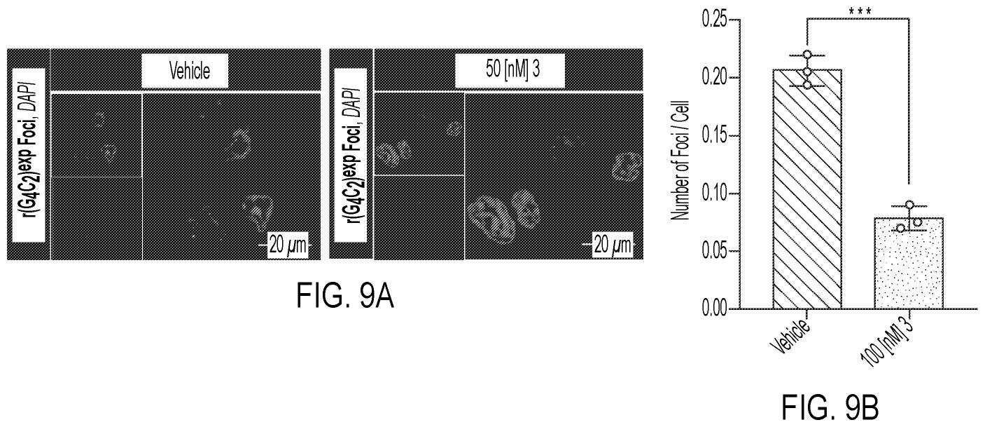

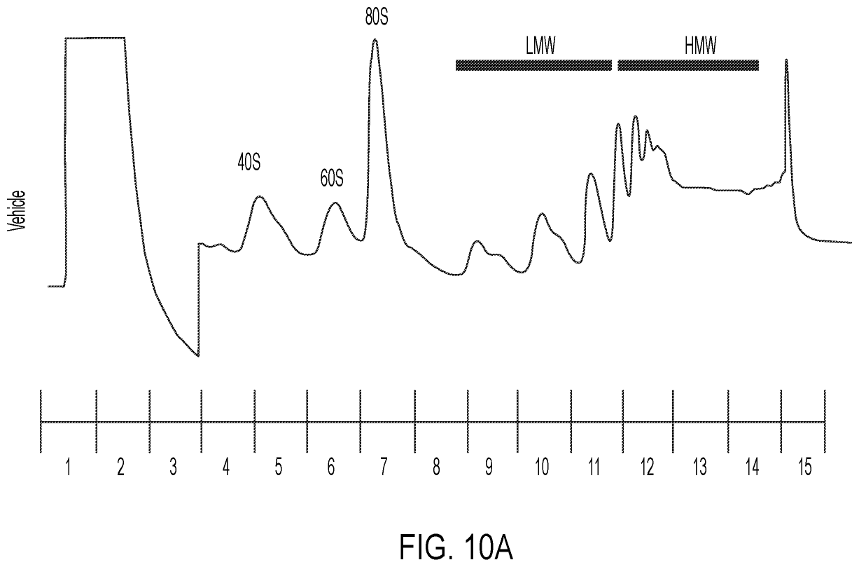

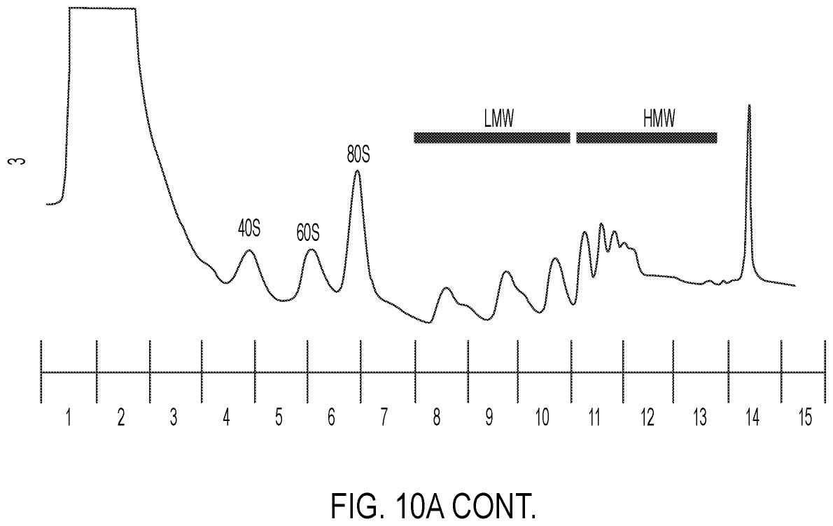

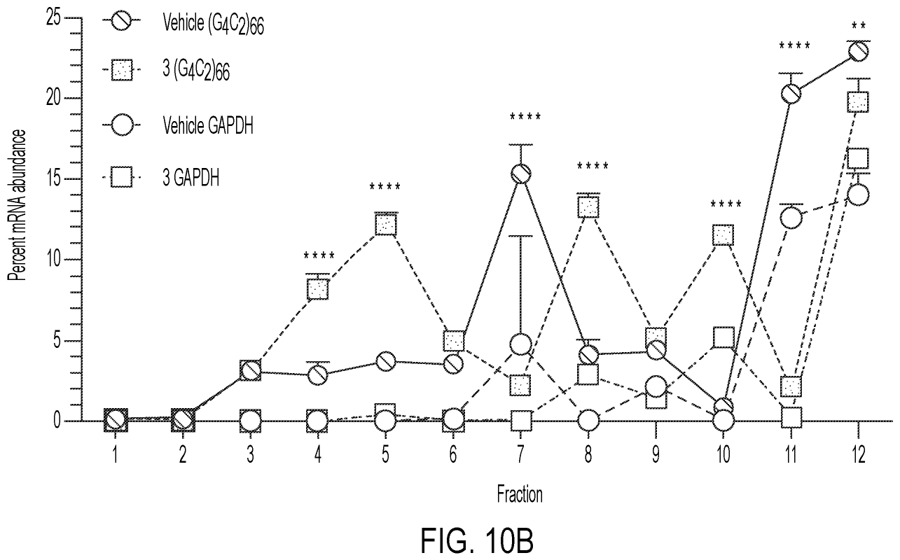

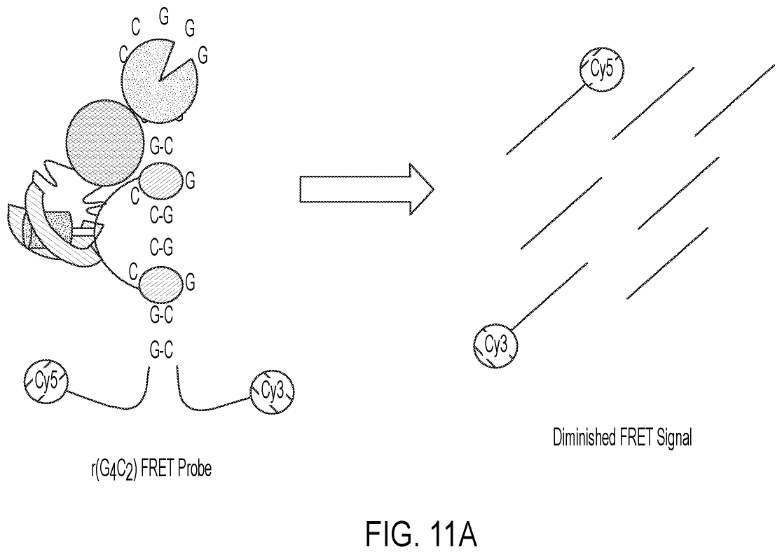

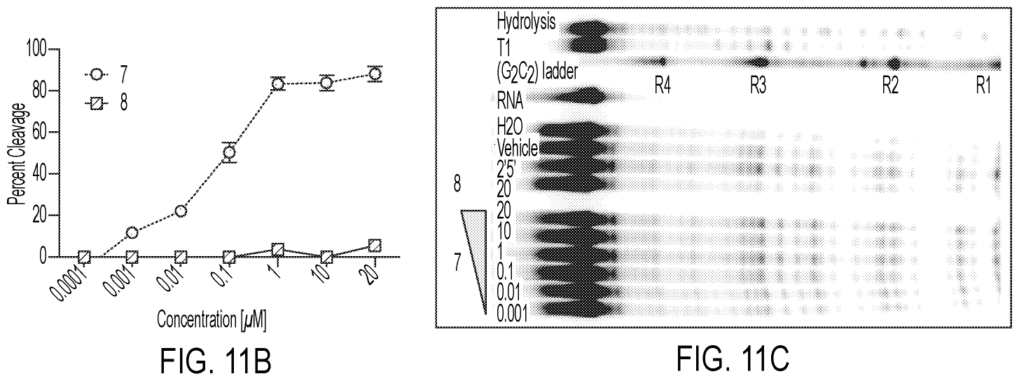

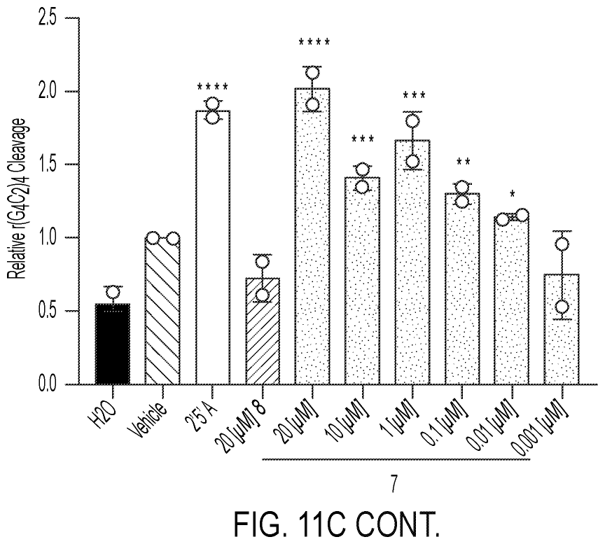

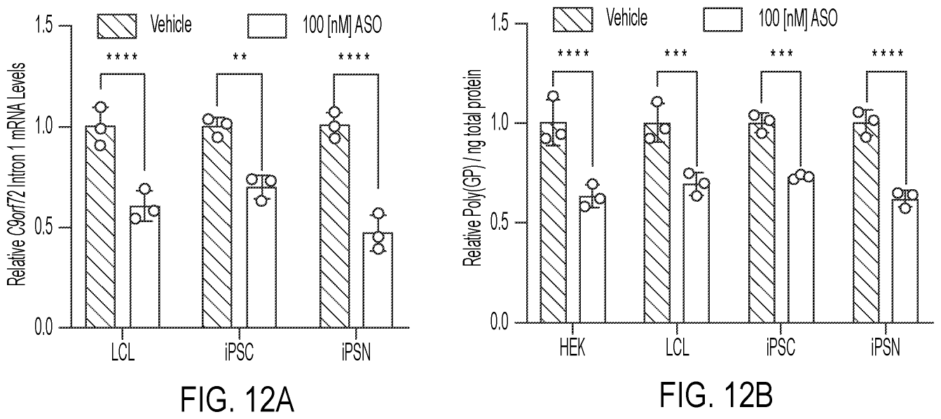

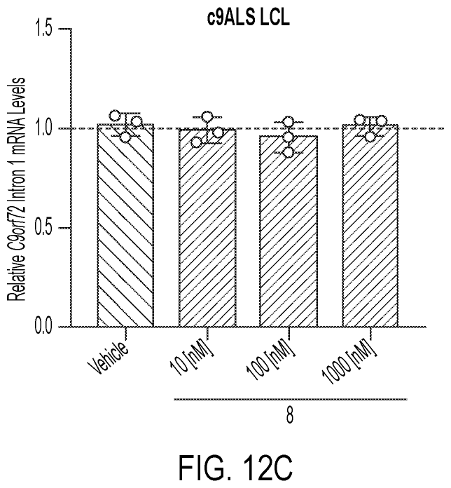

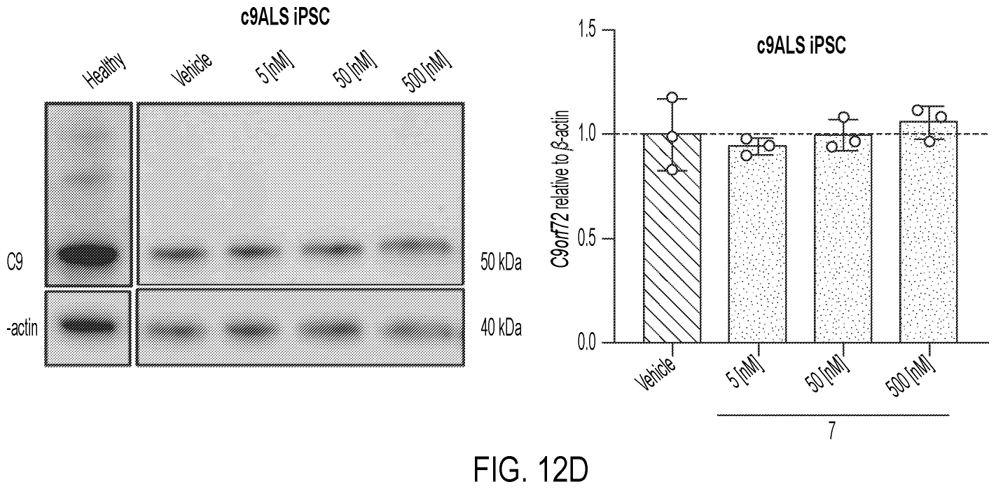

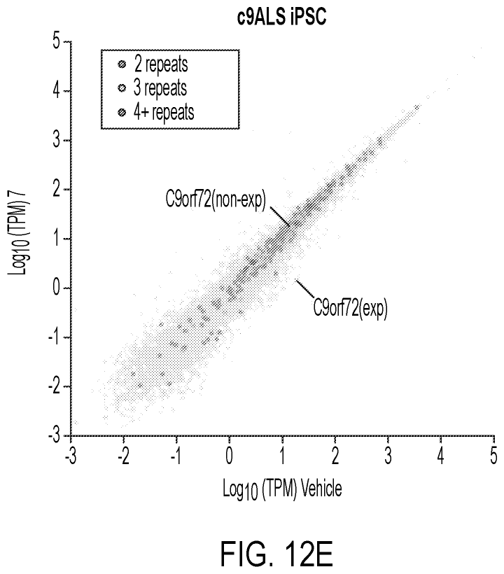

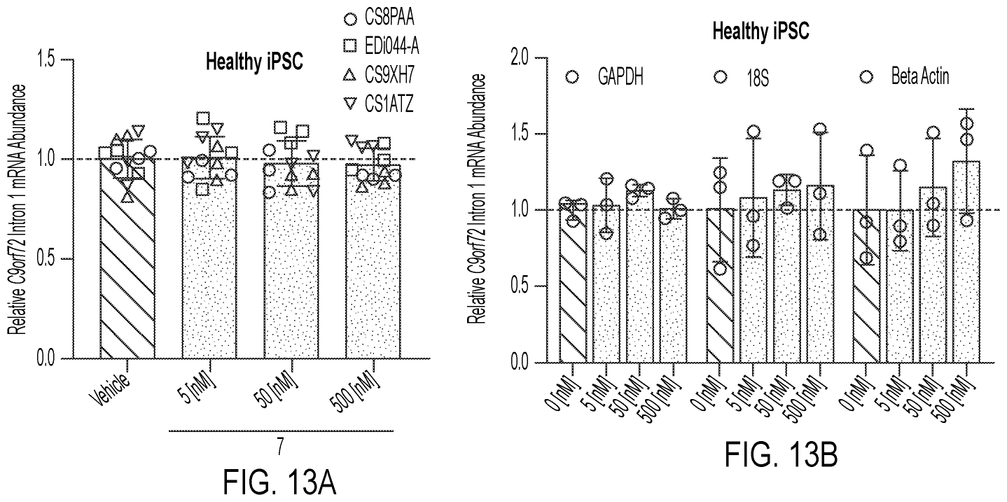

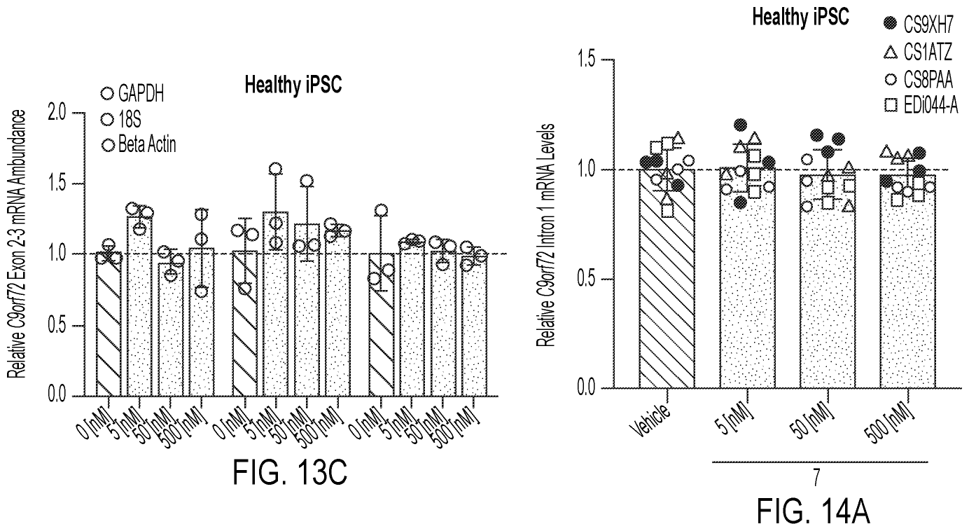

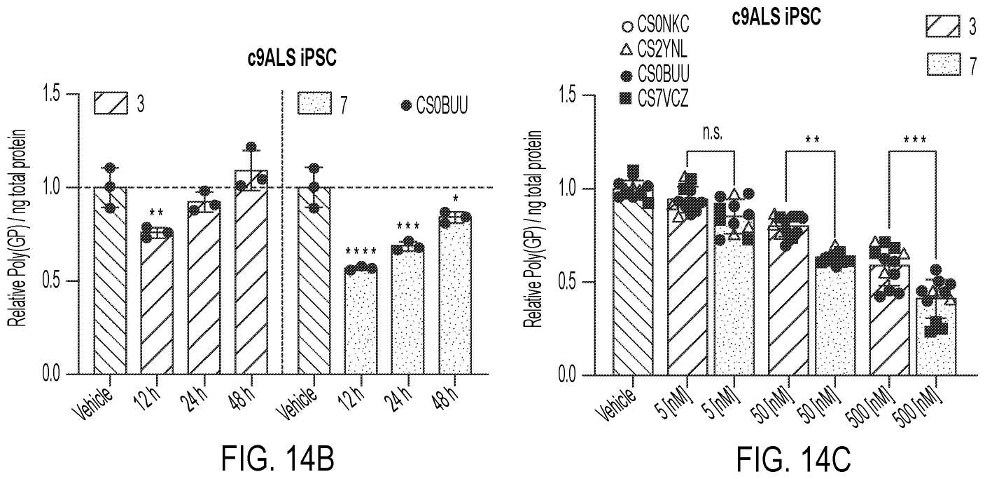

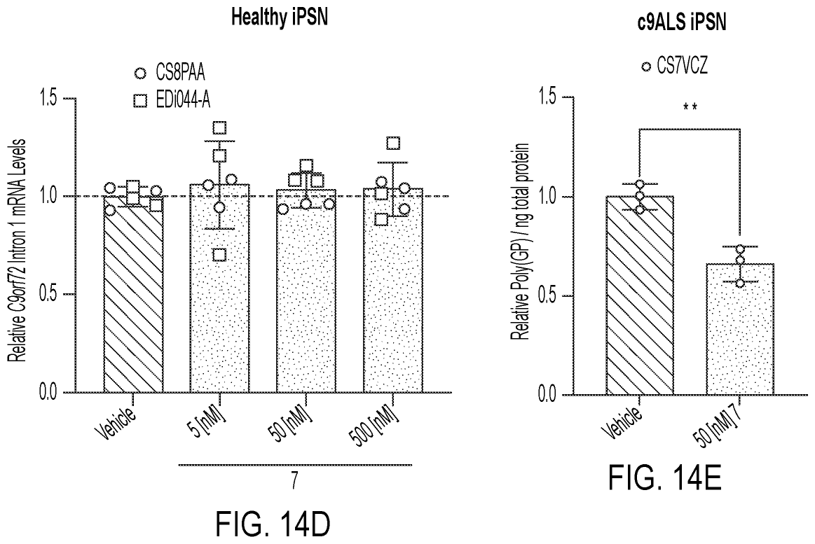

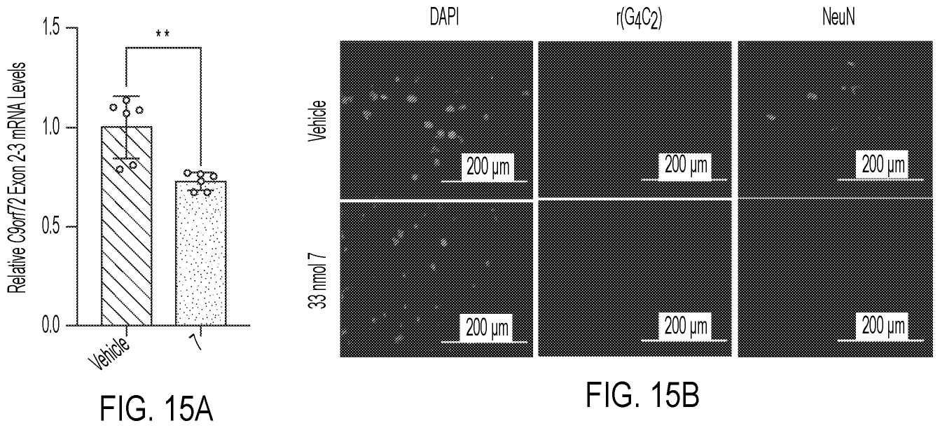

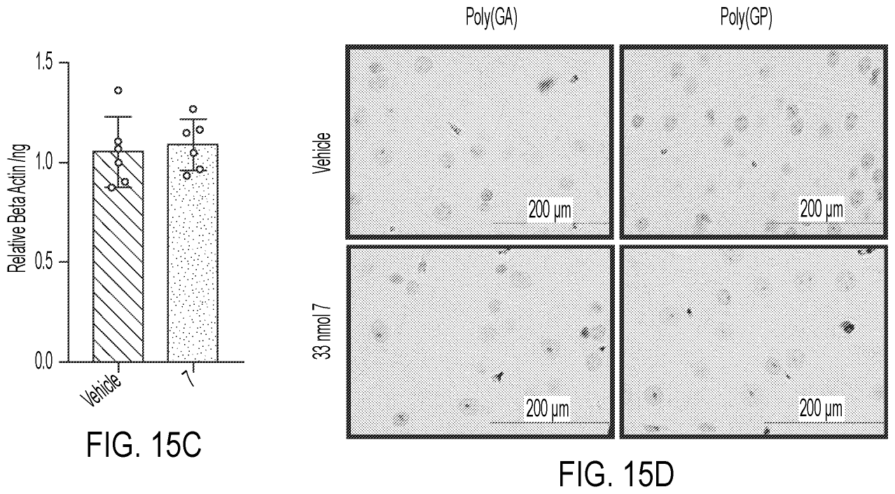

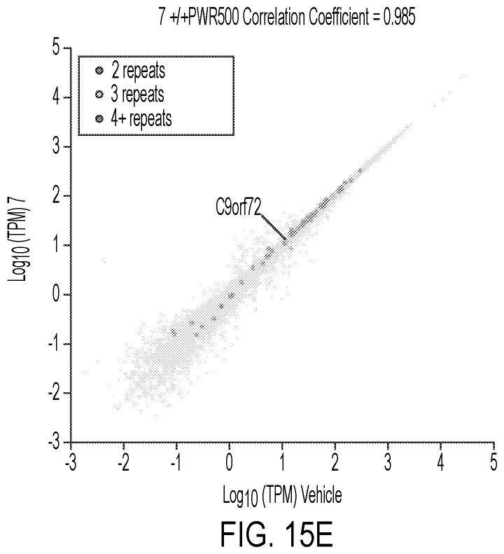

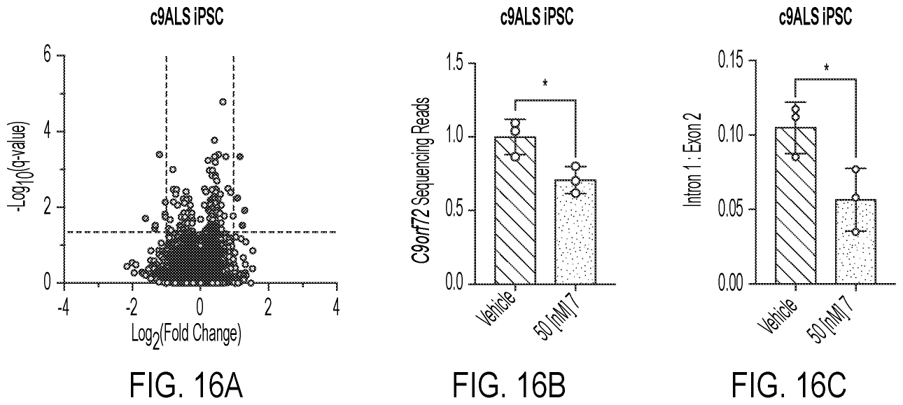

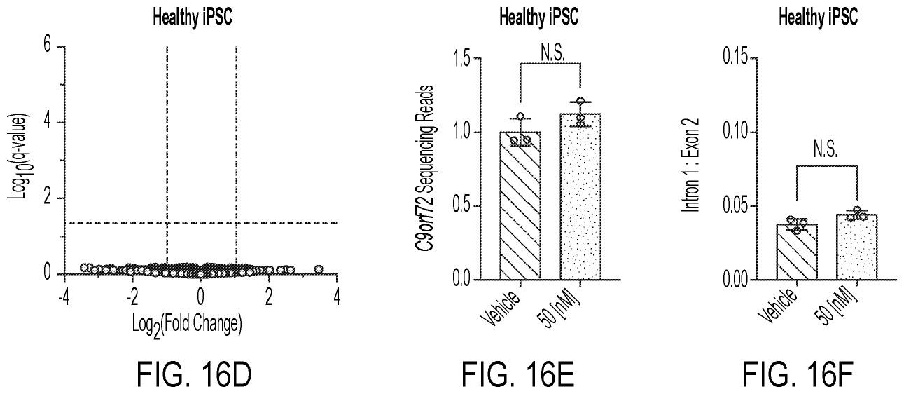

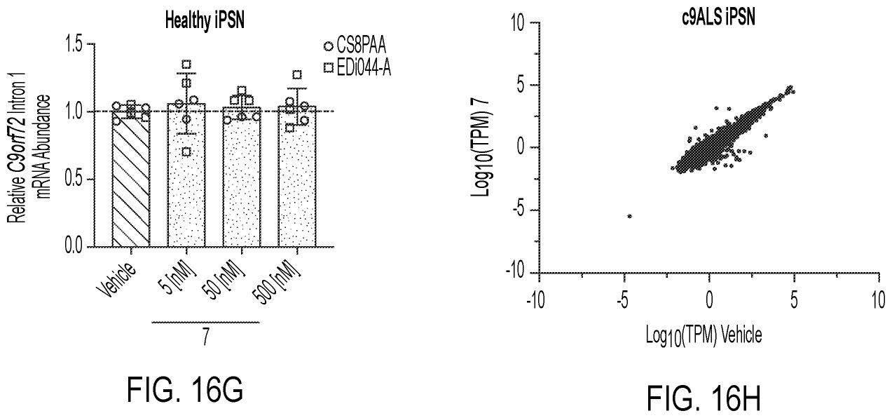

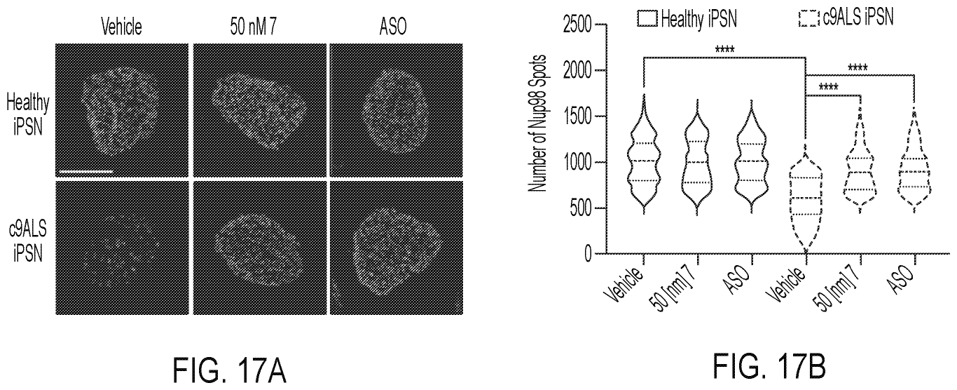

Based on these data, a library of dimers was synthesized that display two copies of the RNA-binding module, conjugated to polyethylene glycol (PEG, i.e. polyoxyethylenyl) linkers of different lengths via position 9 (Experimental Section below). To identify the PEG scaffold with optimal display of 1, that is, the same distance separating the loops in r(G₄C₂)^{exp}, their potencies for inhibiting the binding of heterogenous nuclear ribonucleoprotein H1 (hnRNP H1) in vitro was measured by facile time-resolved fluorescence resonance energy transfer (TR-FRET) assay (12). The most potent dimer, 2 (ALS compound Formula II, Y is oxygen, a is 2, FIG. 1-D) had an IC₅₀ of ˜1.8 μM, 10-fold more potent than 1 (IC₅₀˜19 μM) (12) and a K_{d} of 50±8 nM, as measured by BLI (Table 1). The selectivity of 2 for related oligonucleotides, d(G₄C₂)₈, r(G₂C₂)₁₀ (a hairpin with a fully paired stem), and r(G₂C₄)₈, which models the C9orf72 antisense repeat expansion, ranged from ˜26-fold to ˜640-fold (FIG. 7-A & Table 1). To further increase the binding affinity of 2 and provide a chemical handle for further functionalization, we replaced the oxygen atom within the ethyleneglycol linker with an NH group to generate 3 (ALS compound Formula II, Y is NH, a is 2, FIG. 1-D). Indeed, 3 bound ˜13-fold more avidly to r(G₄C₂)₈ than 2 with a K_{d} of 4±0.07 nM (Table 1). Notably, 3 had a slower dissociation rate (k_{off}) compared to 2, providing longer residence time on the target RNA.

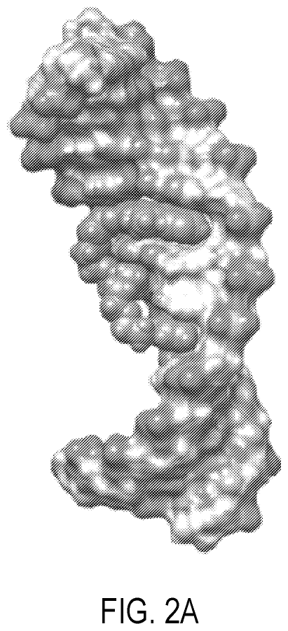

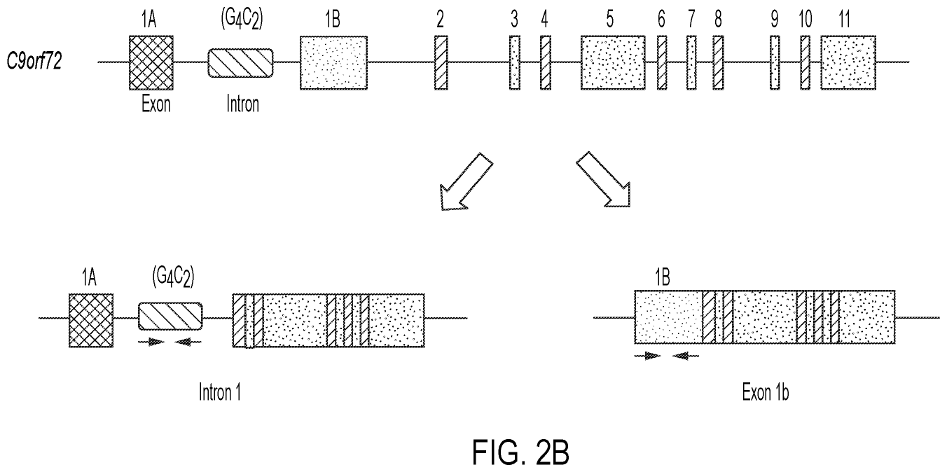

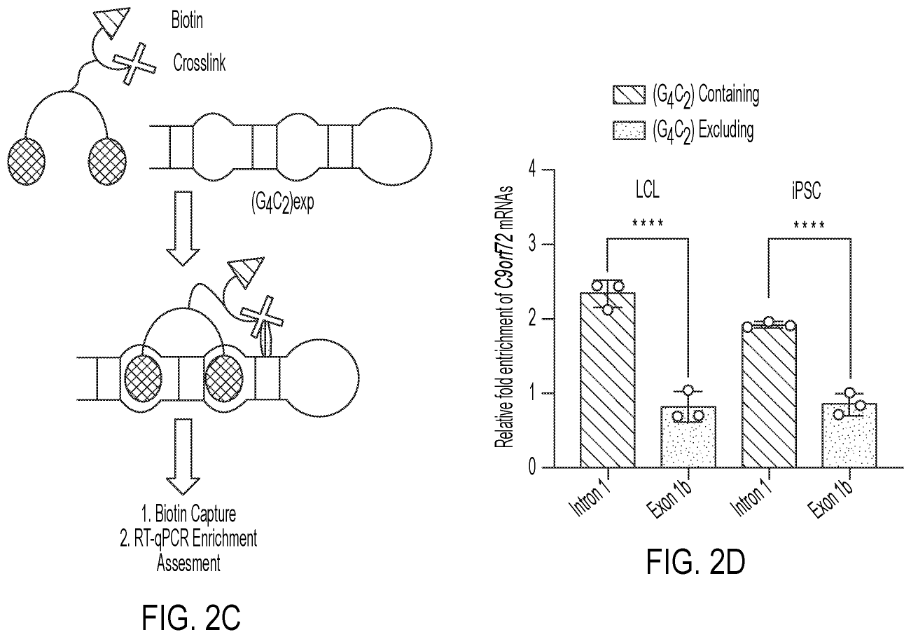

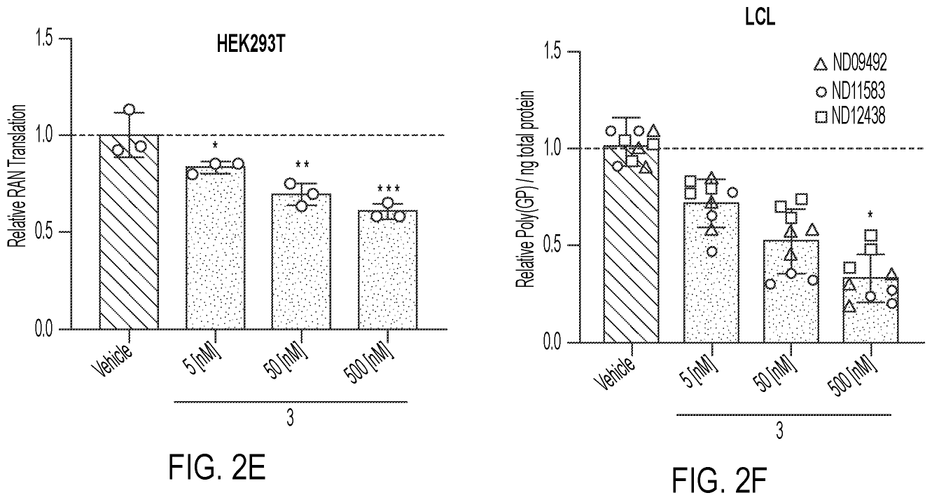

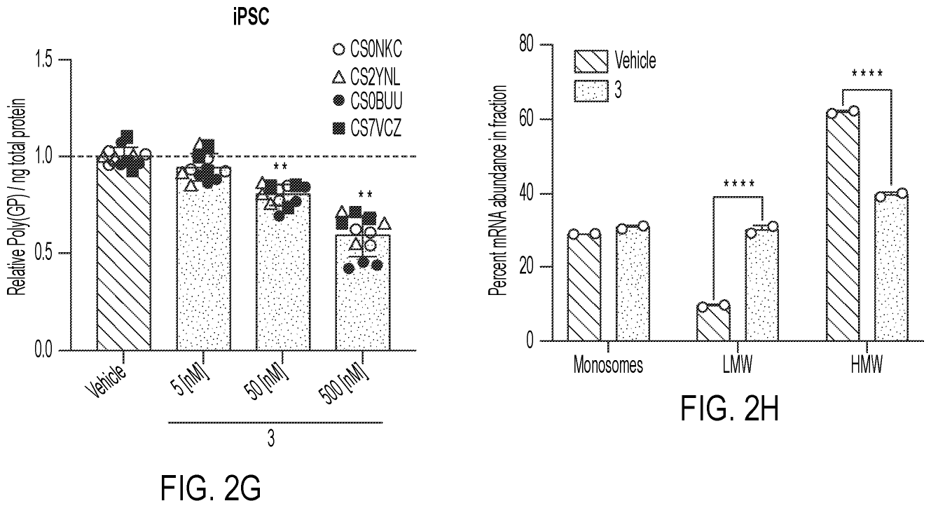

Like 2, 3 was also selective for r(G₄C₂)₈ over d(G₄C₂)₈ (˜225-fold), r(G₂C₂)₁₀ (˜200-fold), and r(G₂C₄)₈ (˜10-fold). This increase in avidity also increased its potency in the TR-FRET assay with an IC₅₀ of 0.9±0.1 μM (Table 1). Complex formation between 3 and r(G₄C₂)₄ was verified by monitoring the imino proton region via 1D NMR, in which two imino proton peaks emerged at 10.5 ppm, where non-canonically paired G residues appear (FIG. 7-B). Notably, no changes were observed in the 1D NMR spectrum of r(G₂C₂)₄, a hairpin with a fully paired stem, upon addition of 3 (FIG. 7-C). The 2D NOE spectra and completed molecular dynamics simulations (MD) were analyzed to generate a 3D model of 3 bound to the repeats (FIGS. 2-A & 7-D). The 3-r(G₄C₂) repeat complex was primarily stabilized by stacking interactions between each RNA binding module and the 1×1 nucleotide GG internal loop and a closing GC pair of each loop. Methyl groups on the ellipticines (carbazole tricyclic group) fill cavities in the major and minor grooves of the RNA, further stabilizing the complex via van der Waals interactions (FIGS. 2-A & 7-D).

To validate engagement of r(G₄C₂)^{exp} by 3 in vitro and in cellulis, a method developed in the Disney laboratory dubbed Chemical Cross-Linking and Isolation Pull-down (Chem-CLIP) and its competitive (C) variant (C-Chem-CLIP) (13, 18-20) was employed. These approaches enable target engagement studies in cells, cross-linking a small molecule to its target(s) followed by the subsequent isolation and analysis of the small molecule-RNA adducts. Compound 3 was functionalized to contain a diazirine cross-linking module (Formula IV, summary section), which upon exposure to light reacts with proximal targets, to afford Chem-CLIP probe 4 (ALS compound of Formulas II and IV); a control Chem-CLIP probe lacking RNA-binding modules, 5 (Formula IV with amide N bound to H and propyl), was also synthesized (See FIGS. 7-E and F). In vitro, 4 pulled down r(G₄C₂)₈ dose-dependently, with ˜40% of the RNA pulled down with 500 nM compound (FIG. 7-G). In contrast, control Chem-CLIP probe 5 only pulled down <25% RNA at all concentrations tested, up to 10 μM (FIG. 7-G). To confirm that 3 and 4 bind the same site within r(G₄C₂)₈, we completed C-Chem-CLIP studies with a constant concentration of Chem-CLIP probe 4 (500 nM) and increasing concentrations of 3. Indeed, 3 reduced the percentage of RNA pulled down in a dose dependent fashion (FIG. 7-G). Notably, 4 did not statistically significantly pull-down other nucleic acids, including the quadruplex form of r(G₄C₂) repeats (by addition of K⁺), base paired r(G₂C₂)₁₀, d(G₄C₂)₈, and d(G₂C₂)₁₀ more than 5 (FIG. 7-G). These results indicate that the observed pull-down of these other targets by 4 is due to the non-selective reaction of the diazirine.

Target engagement in ALS patient-derived LCLs and iPSCs, which both possess expanded G₄C₂ repeats, was next studied. When the mutant C9orf72 allele is processed, it produces variants that: (i) contain exon 1A and intron 1, which harbors r(G₄C₂)^{exp}; or (ii) lack exon 1A and intron 1 but contain exon 1B (FIG. 2-B). Therefore, we used two sets of RT-qPCR primers to determine if 4 selectively cross-links to variants containing the repeat expansion (FIG. 2-C). In both C9orf72 ALS patient-derived lymphoblastoid cells and iPSCs, 4 selectively pulled down variants with r(G₄C₂)^{exp}, enriching the RNA levels by 1.9±0.4 and 2.3±0.2 fold, respectively, while no enrichment was observed in variants that do not contain the repeat expansion (FIG. 2-D).

To further validate 3's binding site (3 is ALS compound of Formula II with R as H), we used a method developed in our lab named ASO-Bind-Map, which uses competition between antisense oligonucleotides (ASOs) and a small molecule to precisely define the small molecule's binding site within an RNA target (21, 22). That is, a small molecule that binds and stabilizes the RNA target will prevent binding of the ASO and hence its subsequent degradation by Rnase H. We treated patient-derived LCLs with 3 followed by transfection of an oligonucleotide complementary to r(G₄C₂)^{exp} [5′-mG*mG*mC*C*C*C*G*G*C*C*C*C*G*G*C*C*C*mC*mG*mG, where m indicates a 2′-O-methyl residue and * indicates a locked nucleic acid (LNA) residue]. This ASO is different from a previously reported ASOs that target the intron upstream of the repeat [5′-CCCGGCCCCTAGCGCGCGAC; 5′-GCCTTACTCTAGGACCAAGA; or TACAGGCTGCGGTTGTTTCC (Ionis)] (11, 23-25). As expected, a dose-dependent increase in the intron 1 levels of C9orf72 mRNA was observed, verifying that 3 competes with the complementary ASO for the r(G₄C₂)^{exp} repeat (FIG. 7-H). Collectively, each of these target engagement studies show that 3 binds r(G₄C₂)^{exp} in patient-derived cells.

Compound 3 (ALS compound of Formula II, R═H) Inhibits RAN Translation in c9ALS/FTD Patient-Derived Cells.

The target engagement studies showed that, 3 binds r(G₄C₂)^{exp}, as designed, in ALS patient-derived cells. To study if 3 can selectively modulate RAN translation, HEK293T cells were co-transfected with either a plasmid expressing (G₄C₂)₆₆-No ATG-Nano-luciferase, which solely undergoes RAN translation (12), and a plasmid encoding SV40-Firefly luciferase. Notably, 3 inhibited RAN translation of r(G₄C₂)₆₆ (as measured by reduction of Nano-luciferase activity); FIG. 2-E) without affecting r(G₄C₂)₆₆ levels, i.e., 3 did not inhibit transcription (FIG. 8-A). Consistent with its lack of effect on C9orf72 mRNA levels, 3 did not inhibit topoisomerase activity in vitro, indicating it does not bind DNA and further supporting specific engagement of r(G₄C₂)^{exp} (FIG. 8-B).

Patient-derived LCLs were next treated with 3 at 5, 50, and 500 nM for 4 days and then measured Poly(GP) abundance by an electroluminescent sandwich immunoassay. A dose-dependent decrease in Poly(GP) expression, significant at all concentrations tested, was observed (FIG. 2-F). Notably, compound 3 had no effect on expression levels of C9orf72 mRNA variants harboring the HRE, as determined by RT-qPCR with primers specific for intron 1 where r(G₄C₂)^{exp} resides (FIG. 8-A). Additionally, 3 also reduced the number of r(G₄C₂)^{exp}-containing nuclear foci in patient-derived LCLs by ˜60% upon treatment with 100 nM of 3 (FIG. 9).

In agreement with these observations, 3 also reduced Poly(GP) levels dose-dependently in iPSCs from c9ALS patients (FIGS. 2-G & 8-E), with no effect on C9orf72 intron 1 levels (FIG. 8-A) or wild-type (WT) C9ORF72 protein, as determined by Western blot (FIG. 8-C). [Notably, no toxicity was observed in c9 patient-derived iPSCs treated with 3 over a range of concentrations (1-1000 nM; FIG. 8-D).] Collectively, these data indicate that only RAN translation but not canonical translation, was inhibited by compound treatment.

### Compound 3's Mode of Action, Inhibiting Ribosome Loading.

The data showing that 3 did not reduce C9orf72 intron 1 or WT C9ORF72 protein levels in patient-derived cells (FIG. 2, E-G & FIG. 8, A) suggest that the compound either inhibits ribosome binding of r(G₄C₂)^{exp} or induces ribosome stalling on r(G₄C₂)^{exp}, both of which can be explored by polysome profiling (26, 27). Notably, r(G₄C₂)^{exp} causes retention of C9orf72 intron 1 (28). Whether 3 binds r(G₄C₂)^{exp} in the context of the entire transcript or the spliced out intron, it inhibits RAN translation and not canonical translation (FIG. 8-C) nor did it affect overall C9orf72 mRNA levels or intron 1 levels (FIGS. 8-A and E).

To gain insight into the mechanism of action of 3, HEK293T cells were transfected with a plasmid that encodes r(G₄C₂)₆₆-No ATG-GFP and performed polysome profiling. Treatment of HEK293T cells expressing r(G₄C₂)₆₆ with 3 reduced the amount of the transcript loaded into high-molecular-weight (HMW) polysomes fractions by 22±2% and increased the its abundance in low-molecular-weight (LMW) fractions by 17±1% (FIGS. 2-H and 10-A & B). Recent studies suggested that blocking formation of HMW polysomes leads to inhibition of RAN translation of r(G₄C₂)^{exp} (29). Importantly, 3 had no effect on polysome loading of GAPDH (control transcript) under the same experimental conditions (FIGS. 2-H and 10-A & B). Previous studies have found that small molecules targeting r(CGG)^{exp} similarly block loading of ribosomes onto the repeat mRNA, thus inhibiting RAN translation but not canonical translation (30).

Having validated the lead, binding compound 3 for selectively reducing C9orf72 HRE-associated pathologies and defining its mechanism of action, the next step for development of an ALS treatment of humans focuses on improvement of potency and selectivity such that compound 3 could be studied in a preclinical in vivo model of c9ALS/FTD.

Design and in vitro Validation of a r(G₄C₂)^{exp}-Targeting RIBOTAC.

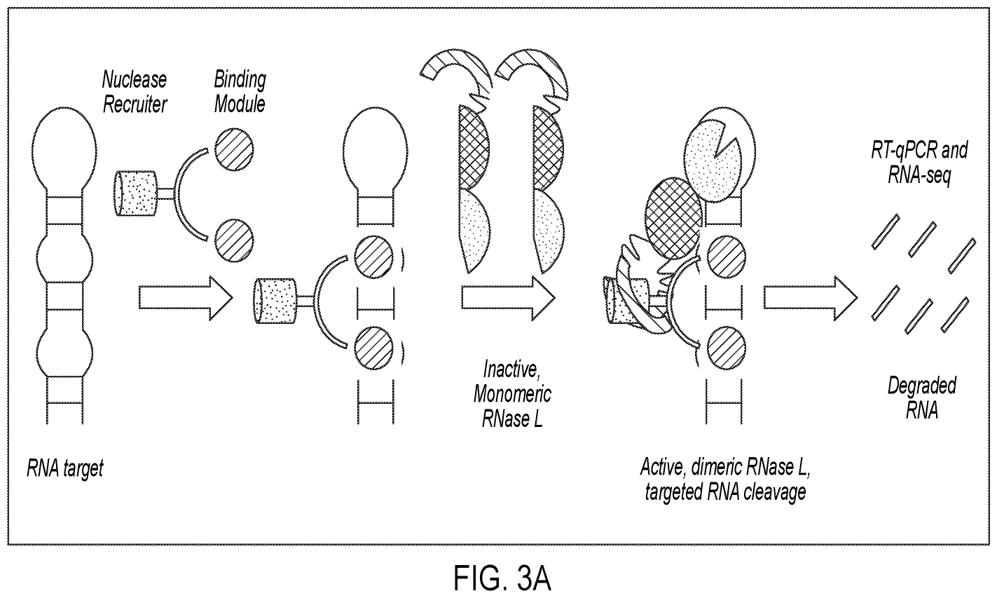

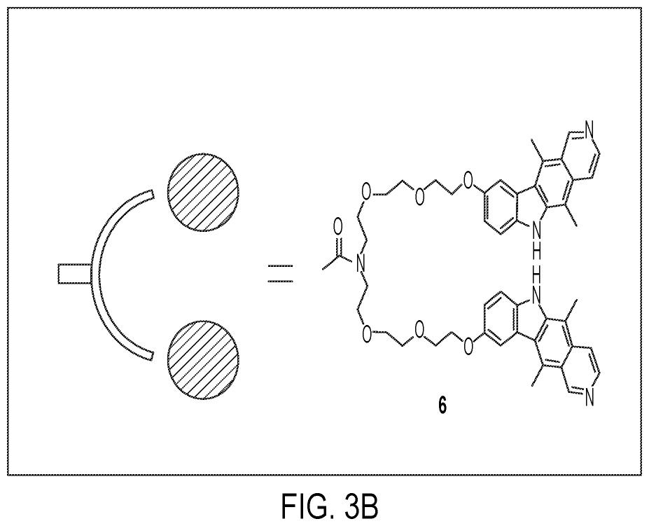

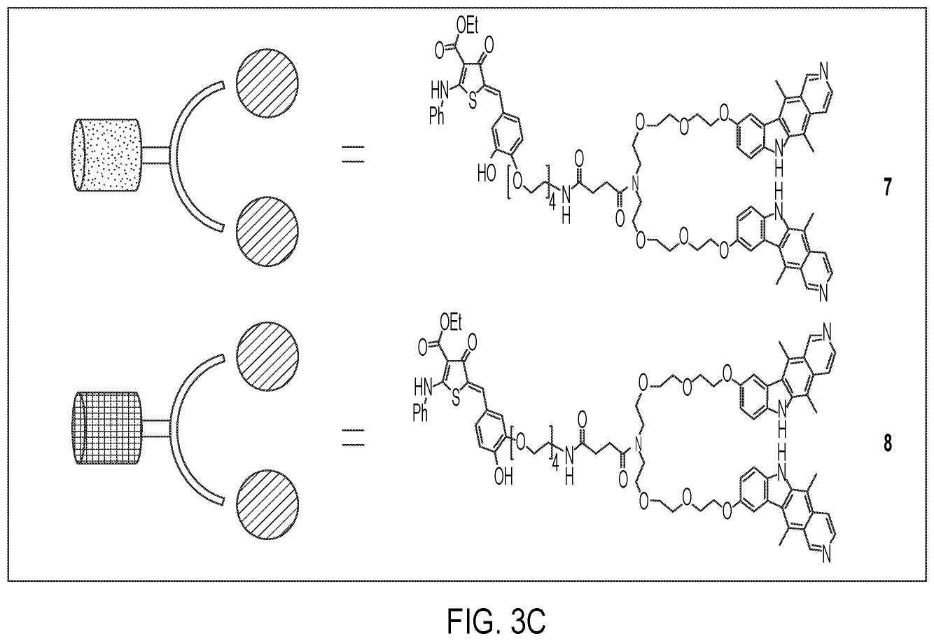

The Disney laboratory has developed a new class of small molecules, dubbed ribonuclease recruiting chimeras (RIBOTACs) that potently and selectively cleave an RNA target by recruiting an endogenous nuclease (FIG. 3-A) (14, 31, 32). Using this approach, the Disney laboratory designed a r(G₄C₂)^{exp}-targeting RIBOTAC using the small molecule Rnase L-recruiter ethyl (Z)-5-(3-hydroxy-4-methoxybenzylidene)-4-oxo-2-(phenylamino)-4,5-dihydrothiophene-3-carboxylate (Formula III with ethoxy replacing the N) (14), which was optimized from a previously reported small molecule (33). Rnase L, or latent Rnase, is expressed in minute quantities as an inactive monomeric subunit in all cells. It is dimerized and activated by 2′-5′poly(A), which is synthesized in response to viral infections (34). In contrast, the Disney RIBOTAC locally recruits Rnase L selectively to a r(G₄C₂)^{exp}, increasing its local, effective concentration to trigger cleavage (FIG. 3-A). This approach will interface RNA targets with RNA quality control pathways in a programmable manner to allow systematic elimination of the toxic transcript.

In brief, the Disney laboratory synthesized three compounds: (i) 6, in which 3's linker's amino group was acetylated (the ALS compound Formula II with Y as R—N and R as acetyl, FIG. 3-B) to study the effects of functionalization; (ii) 7, a r(G₄C₂)^{exp}-targeting RIBOTAC in which the Rnase L-recruiting heterocycle was installed via 3's linker amino group (ALS compound Formulas II and III, FIG. 3-C); and (iii) control compound, 8, in which an inactive Rnase L-recruiting compound was conjugated to 3 (ALS compound Formula II with the meta configured isomer of Formula III, see paragraph 0006 and FIG. 3-C). The effect of modifying 3 on molecular recognition was first studied by evaluating the potency of 6 and 7 (in the absence of Rnase L) for inhibiting r(G₄C₂)₈ and hnRNP H1 complex formation. As expected, the IC₅₀'s of 6 and 7 were similar to 3 being 2.3±0.3 μM, 2.4±0.1 μM, and 0.9±0.1 μM, respectively (Table 1). We next assessed 7's ability to recruit Rnase L and cleave the RNA using a FRET-based assay in which r(G₄C₂)₈ was dually labeled with Cy5 (5′ end) and Cy3 (3′ end) (FIG. 11-A). The addition of 7 and Rnase L dose dependently decreased the FRET signal while no change in FRET signal was observed upon addition of inactive RIBOTAC 8 and Rnase L (FIG. 11-B). Collectively, these results suggest that 7 induced r(G₄C₂)₈ cleavage by recruiting and activating Rnase L. These findings were confirmed using radioactively labeled r(G₄C₂)₈ and analysis by gel electrophoresis (FIG. 11-C).

Activity of a r(G₄C₂)^{exp}-Targeting RIBOTAC in a HEK293T Cellular Model.

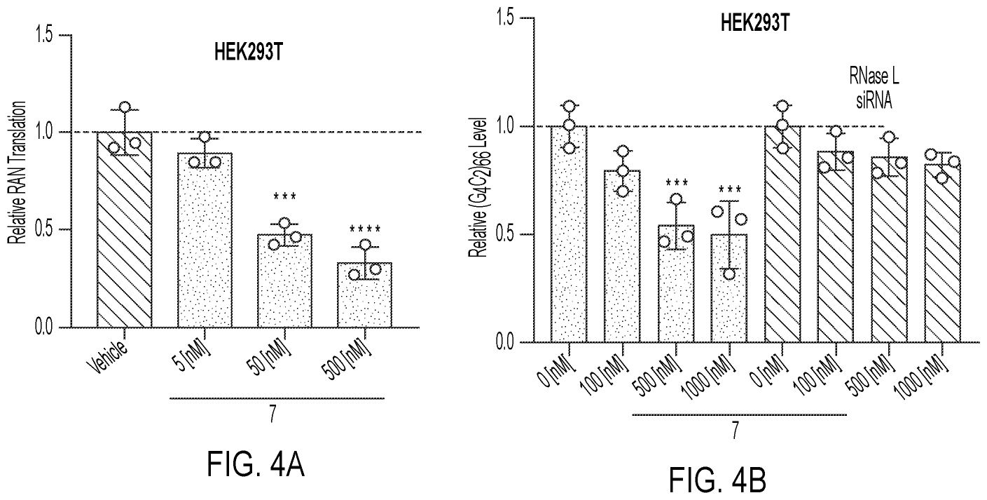

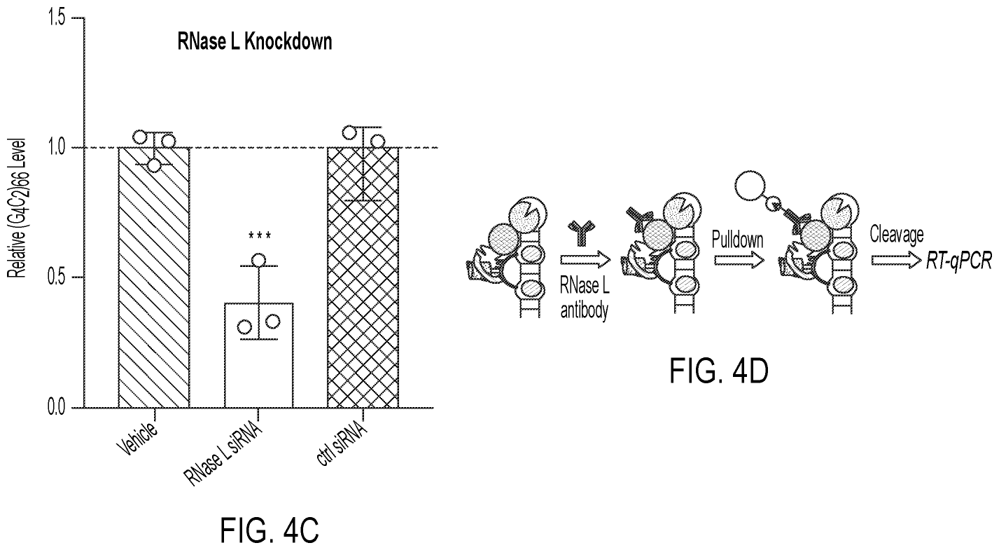

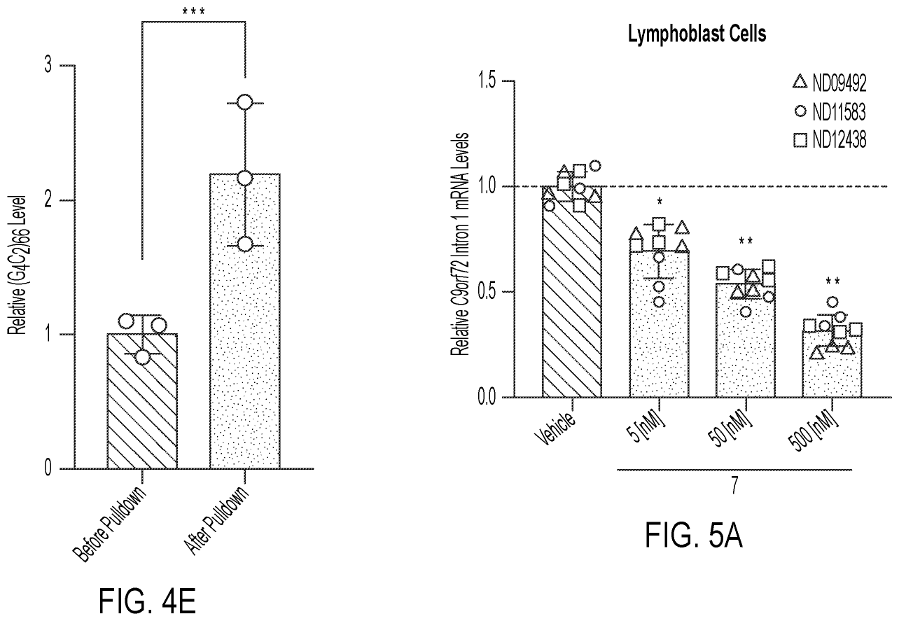

To assess if the r(G₄C₂)^{exp} cleavage observed in vitro translated to cellular activity, compound 7's ability to inhibit RAN translation was assessed in our HEK293T model. HEK293T cells were co-transfected with plasmids encoding (G₄C₂)₆₆-NO ATG-Nano-luciferase (RAN translation) and SV40-Firefly luciferase (control; canonical translation) and treated with 7. Indeed, 7 inhibited RAN translation of r(G₄C₂)₆₆ by ˜65% at a dose of 500 nM (as measured by reduction of Nano-luciferase activity normalized to firefly luciferase activity) (FIG. 4-A). We next characterized 7 in HEK293T cells transfected with the plasmid encoding (G₄C₂)₆₆-No ATG-GFP for reducing levels of the r(G₄C₂)^{exp}-containing transcript. Indeed, 7 reduced levels of the r(G₄C₂)^{exp}-containing mRNA in a dose-dependent fashion, with a 46±2% reduction observed upon treatment with 500 nM compound (FIG. 4-B). Confirming an Rnase L-dependent cleavage of the r(G₄C₂)^{exp}-containing mRNA by 7, it was found that: (i) knock down of Rnase L using an siRNA (by ˜70%), but not a control siRNA, ablated 7's ability to reduce levels of r(G₄C₂)^{exp} (FIGS. 4-B & C); and (ii) the r(G₄C₂)^{exp}-containing mRNA was enriched by ˜2-fold in immunoprecipitated fractions using an anti-Rnase L antibody, as compared to vehicle-treated samples (FIGS. 4-D & E).

r(G₄C₂)^{exp}-Targeting RIBOTAC Rescues Pathological Hallmarks in c9ALS Patient-Derived LCLs.

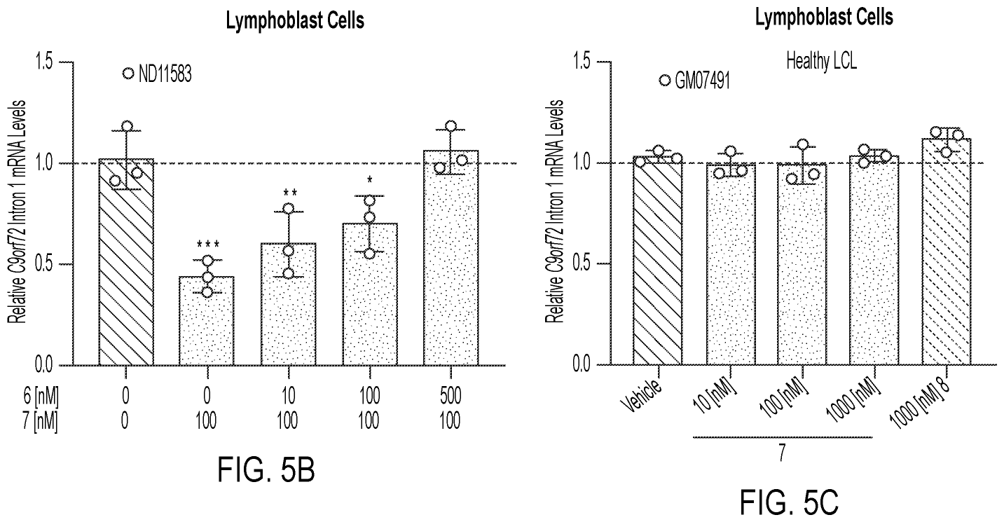

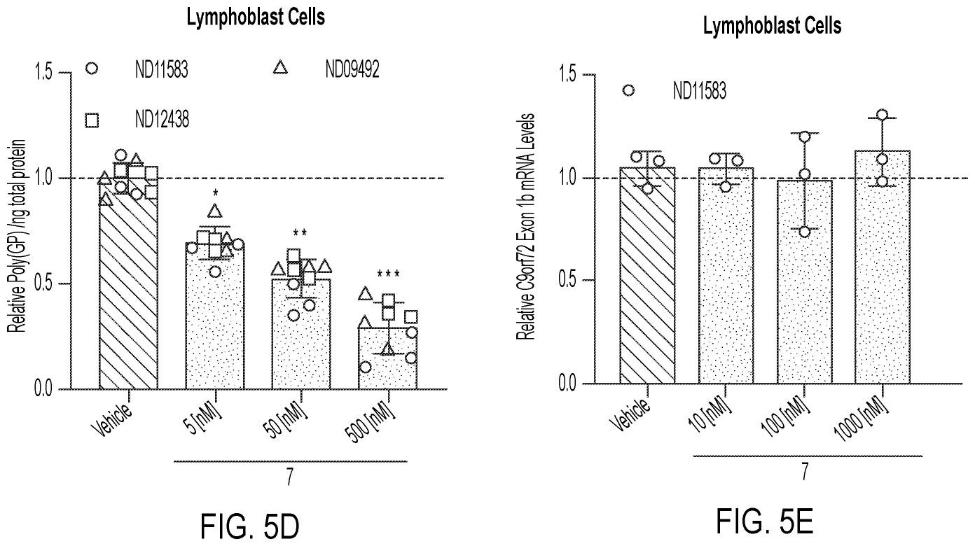

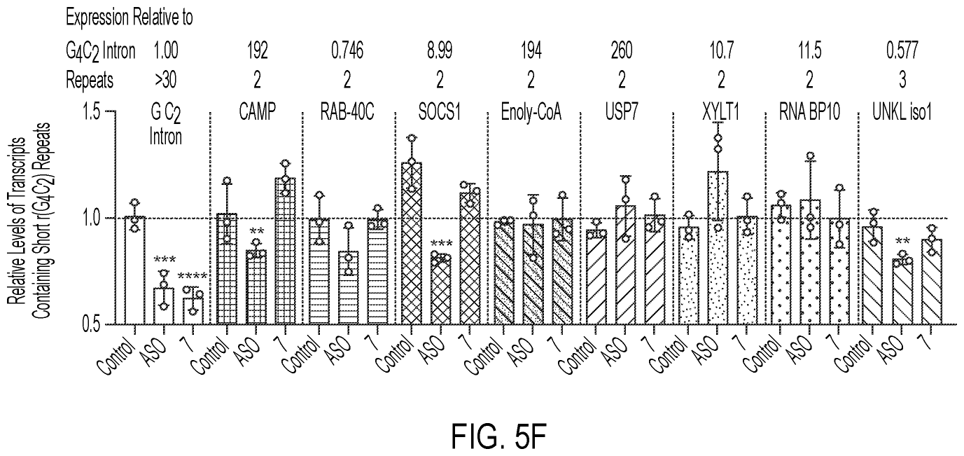

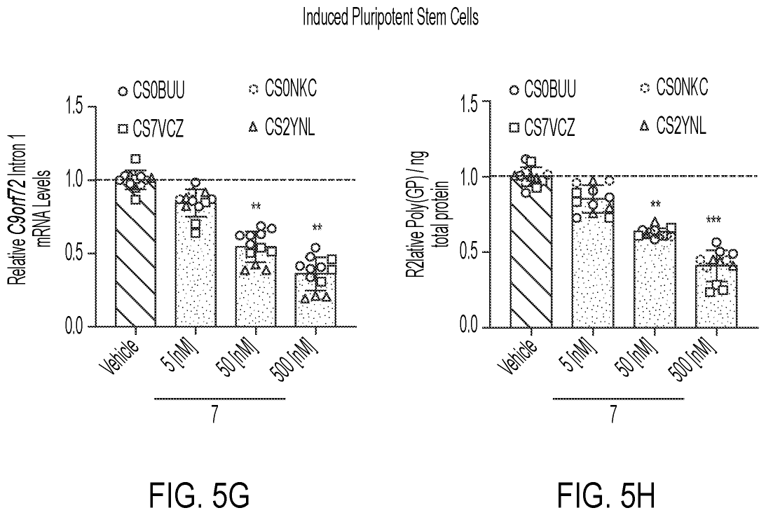

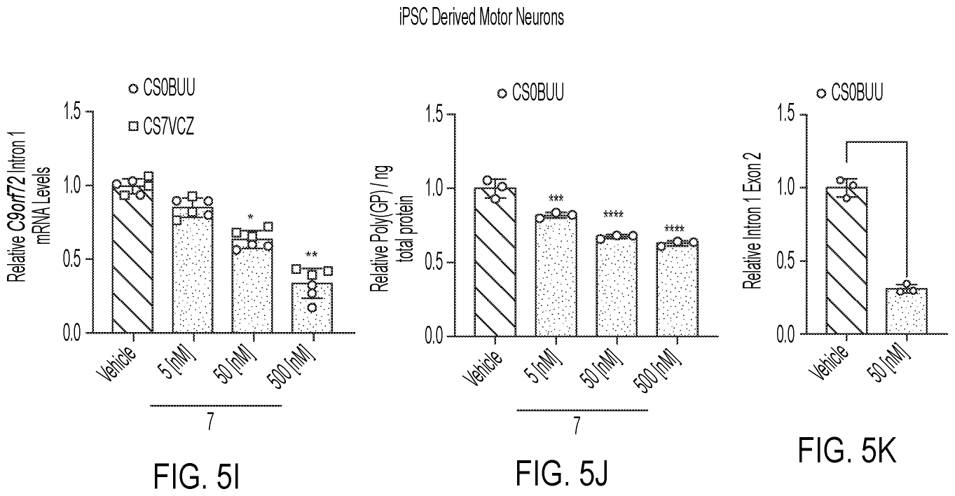

It was next assessed whether 7 could selectively cleave r(G₄C₂)^{exp}P in c9ALS patient-derived LCLs. It was found that 7 reduced C9orf72 intron 1 levels with an IC₅₀ of −50 nM (FIG. 5-A). Furthermore, C9orf72 intron 1 levels were dose-dependently restored when patient-derived LCLs were co-treated with increasing concentrations of 6, which binds but does not cleave the repeat expansion, and a constant concentration of 7 (FIG. 5-B). These experiments confirmed that 6 and 7 compete for the same binding site, r(G₄C₂)^{exp}. Importantly, 7 did not affect C9orf72 intron 1 levels in LCLs from a healthy donor (FIG. 5-C). As expected, control compound 8, which cannot recruit Rnase L, elicited no changes in intron 1 transcript abundance in both disease-affected and healthy LCLs, confirming specificity for the repeat expansion (FIGS. 5-C & 12-C).

The observed decrease in C9orf72 intron 1 levels due to cleavage of r(G₄C₂)^{exp} resulted in reduced abundance of Poly(GP). In particular, 500 nM of 7 reduced Poly(GP) levels by 71±11% (FIG. 5-D). Thus, nuclease recruitment resulted in a 5-fold increase in potency over the parent compound 3 (FIGS. 2-F & 5-D). Notably, the reduction of RAN translated Poly(GP) was selective given that canonically translated WT C9ORF72 protein levels were unchanged by treatment with 7, as determined by Western blotting (relative to b-actin and compared to vehicle-treated samples; FIG. 12-D).

These data indicate that 7 only cleaves C9orf72 transcripts containing r(G₄C₂)^{exp}, which are present in intron 1. To determine if this is indeed the case, C9orf72 transcripts were quantified using RT-qPCR primers selective for exon 1b, which is only present in transcripts lacking r(G₄C₂)^{exp}P (FIG. 2-B). In transcripts that lack r(G₄C₂)^{exp}, no decrease in C9orf72 was observed after treatment with 7 at any concentration (FIG. 5-E). To further assess compound selectivity, known genes containing short (i.e., non-pathogenic) r(G₄C₂) repeats were analyzed by RT-qPCR for off-target cleavage. No decrease in abundance was observed for any of these eight transcripts upon treatment with 500 nM of 7 (FIG. 5-F).

r(G₄C₂)^{exp}-Targeting RIBOTAC Rescues Pathological Hallmarks in c9ALS Patient-Derived iPSCs.

These foregoing observations were confirmed by investigation of LCLs in iPSCs derived from c9ALS patients. Indeed, after treatment with 50 and 500 nM of 7, r(G₄C₂)^{exp}-containing intron 1 levels were reduced by 45±11% and 64±10%, respectively (FIG. 5-G). Treatment with 7 had no effect on C9orf72 intron 1 levels in iPSC lines from healthy donors (FIG. 13-A). In agreement with reduced C9orf72 intron 1 levels, a significant reduction in the abundance of Poly(GP) was observed in in patient-derived iPSCs (FIG. 5-H). The reduction of RAN translated poly(GP) was selective given that canonically translated WT C9ORF72 protein abundance was unchanged by treatment with 7, as determined by Western blotting (relative to □-actin and compared to vehicle-treated samples; FIG. 14-A). Notably, nuclease recruitment resulted in a significant increase in potency and a prolonged period of activity for reducing Poly(GP) abundance, as compared to parent compound 3 (FIGS. 14-B & C).

To quantify the selectivity of 7 in c9ALS patient-derived iPSCs, we performed full RNA-sequencing (RNA-seq) analysis after treatment with 50 nM of compound 7 for 4 days. Indeed, abundance of C9orf72 transcripts containing the r(G₄C₂)^{exp}-harboring intron 1 were reduced, with no significant (P>0.05) effect on other transcripts containing short, non-pathogenic r(G₄C₂) repeats (FIG. 15, A-C). Further, no changes in the transcriptome were observed in iPSCs derived from healthy donors (FIG. 15-D) nor was there an effect on C9orf72 expression (FIGS. 15-E & F).

r(G₄C₂)^{exp}-Targeting RIBOTAC Rescues Pathological Hallmarks in c9ALS Patient-Derived iPSNs.

The study of 7 was next directed to c9ALS iPSC-derived spinal neurons (iSPNs), which recapitulate the genetic, transcriptional, and biochemical signatures of c9ALS patient brain tissue (24, 35). After treatment with 50 or 500 nM of 7, r(G₄C₂)^{exp}-containing intron 1 abundance was reduced by 45±11% and 64±10%, respectively (FIG. 5-G). The observed decrease in intron 1 abundance as well as the mature transcript, as assessed using RT-qPCR primers that amplify the exon 2-3 junction, was observed whether normalized to GAPDH, 18S, or b-actin housekeeping genes (FIG. 11, G-H). Importantly, treatment with 7 had no effect on C9orf72 intron 1 quantities in iPSC lines from healthy donors (FIG. 13, A-C). In agreement with reduced C9orf72 intron 1 abundance, we also observed a significant (P<0.05) reduction in the abundance of poly(GP) (FIG. 5-H). The reduction of RAN translated poly(GP) was selective given that canonically translated WT C9ORF72 protein abundance was unchanged by treatment with 7, as determined by Western blotting (relative to b-actin and compared to vehicle-treated samples; FIG. 14-A). Nuclease recruitment resulted in a significant (P<0.05) increase in potency and a prolonged duration of activity for reducing poly(GP) abundance, as compared to parent compound 3 (FIGS. 14-B & C).

To quantify the selectivity of 7 in c9ALS patient-derived iPSCs, we performed full RNA-sequencing (RNA-seq) analysis after treatment with 50 nM of compound 7 for 4 days. Indeed, abundance of C9orf72 transcripts containing the r(G₄C₂)^{exp}-harboring intron 1 were reduced, with no significant (P>0.05) effect on other transcripts containing short, non-pathogenic r(G₄C₂) repeats (FIG. 16, A-C). Further, no changes in the transcriptome were observed in iPSCs derived from healthy donors (FIG. 16-D) nor was there an effect on C9orf72 expression (FIGS. 16-E & F).

Previous studies have established a link between C9orf72-mediated ALS/FTD and defects in the nuclear pore complex (NPC) (36, 37). In particular, a recent study identified several nuclear pore proteins to be reduced in C9orf72 neurons, reversible upon ASO treatment (37). Using super-resolution structured illumination microscopy (SIM), it was found that treatment of patient-derived iPSNs with 50 nM of 7 reversed the loss of Nup98, a component of the central channel of the NPC involved in bidirectional transport previously reported in c9ALS iPSNs (38), mirroring the abundance found in healthy control iPSNs (FIG. 16, D-G).

r(G₄C₂)^{exp}-Targeting RIBOTAC Ameliorates c9ALS/FTD Pathology in vivo.

Finally, the therapeutic potential of 7 was determined in a C9orf72 BAC mouse model (+/+PWR500) developed by the Ranum laboratory, which expresses 500 r(G₄C₂) repeats and is dubbed +/+PWR500 (36). These mice exhibit several pathological hallmarks associated with c9ALS/FTD including formation of RNA foci and increased abundance of DPRs (36).

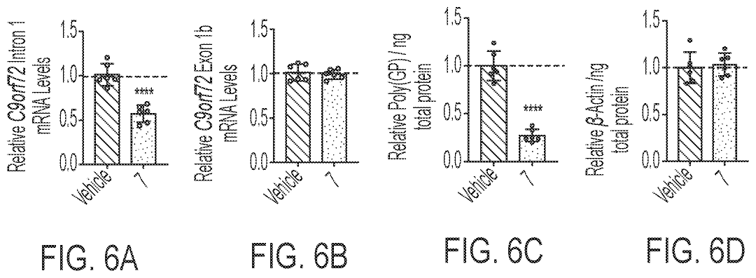

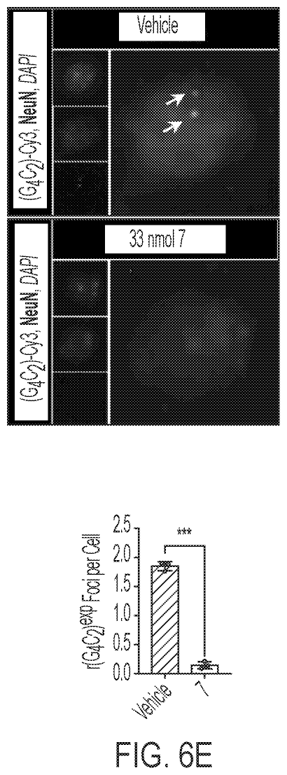

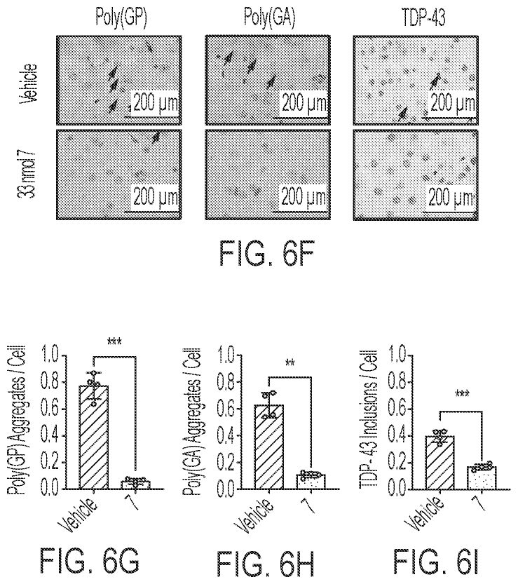

Mice were treated with RIBOTAC 7 (33 nmol) by a single intracerebroventricular (ICV) injection for three weeks. Post treatment, levels of the r(G₄C₂)^{exp}-containing intron 1 mRNA were measured by RT-qPCR, revealing a reduction of 44±22% compared to vehicle-treated controls (FIG. 6-A). Importantly, C9orf72 exon 1b RNA levels, which do not contain the repeat expansion, were not altered (FIG. 6-B). Levels of all C9orf72 transcripts were measured following treatment with 7 (using RT-qPCR primers for the exon 2-exon 3 junction common to all transcripts; FIG. 2-A). Indeed, C9orf72 transcript levels were reduced by 7 to an extent that reflects the percentage of transcripts that contain intron 1 and the amount by which it was reduced by treatment (FIG. 15-A). Collectively, these data suggest target cleavage by the compound is specific for the repeat expansion.

As expected, based on the reduction r(G₄C₂)^{exp} levels, the number of r(G₄C₂)^{exp}-containing nuclear foci was also decreased in the brains of +/+PWR500 mice treated with 7 compared to vehicle-treated mice, as determined by RNA fluorescence in situ hybridization (FISH) (FIG. 6-E; FIG. 15-B shows lack of foci by FISH staining in WT mice, whether treated or untreated). No effect was observed on the abundance of antisense r(G₂C₄)^{exp} foci in treated mice compared to vehicle, demonstrating that compound 7 is selective for the sense transcript (39). Poly(GP) translated from the expanded repeat was also significantly reduced by 74±24% in +/+PWR500 in mice treated with 7 (FIG. 6-C), while no decrease of micro-actin protein expression was observed in either +/+PWR500 mice or WT mice, as determined by the sandwich immunoassay (FIGS. 6-D & 15-C). These data indicate a selective effect on RAN translation, at least for Poly(GP).

An immunohistochemical analysis of sagittal brain sections was undertaken to assess inclusions immunopositive for Poly(GP), Poly(GA) or TAR DNA-binding protein 43 (TDP-43), the latter also a hallmark feature of a subset of cells in c9ALS/FTD (37) (FIG. 6, F-I). A significant decrease in Poly(GP) and Poly(GA) aggregates was seen in both the cortex of treated mice compared to mice that received only vehicle (FIG. 6, F-H), the former consistent with reduction of Poly(GP) levels measured in brain lysates (FIG. 6-C). [Note: Poly(GP) and Poly(GA) were not detected in WT mice, as expected (FIG. 15-D).] TDP-43 inclusions were also decreased throughout the cortex of treated mice relative to vehicle (FIGS. 6, F & I). Collectively, these results demonstrate that the RIBOTAC 7 can rescue c9ALS/FTD pathology in vivo.

### Mechanism of Action and Medical Treatment

In certain embodiments, the invention is directed to methods of inhibiting, suppressing, depressing and/or managing biolevel translation of the aberrant repeat RNA r(G₄C₂)^{exp} associated with amyotrophic lateral sclerosis (ALS) and frontotemporal dementia (FTD). These aberrant RNA repeats are present in cell lines, and patients afflicted with ALS and FTD. The ALS Compounds can reduce translation of the aberrant repeat RNA by binding the repeats or by inducing cleavage of the repeats. The ALS Compounds of Formula II and especially Formula II combined with Formula III as embodiments of the invention for use in the methods disclosed herein bind to the above identified RNA entities and ameliorate and/or inhibit their translation to disease-causing dipeptide repeat proteins.

Embodiments of the Compounds applied in methods of the invention and their pharmaceutical compositions are capable of acting as “inhibitors”, suppressors and or modulators of the above identified RNA entities which means that they are capable of blocking, suppressing or reducing the translation of the RNA entities by simple binding or by facilitating their cleavage. An inhibitor can act with competitive, uncompetitive, or noncompetitive inhibition. An inhibitor can bind reversibly or irreversibly.

The compounds useful for methods of the invention and their pharmaceutical compositions function as therapeutic agents in that they are capable of preventing, ameliorating, modifying and/or affecting a disorder or condition. The characterization of such compounds as therapeutic agents means that, in a statistical sample, the compounds reduce the occurrence of the disorder or condition in the treated sample relative to an untreated control sample or delays the onset or reduces the severity of one or more symptoms of the disorder or condition relative to the untreated control sample.

The ability to prevent, ameliorate, modify and/or affect in relation to a condition, such as a local recurrence (e.g., pain), a disease known as an ALS/FTD disease may be accomplished according to the embodiments of the methods of the invention and includes administration of a composition as described above which reduces, or delays or inhibits or retards the deleterious medical condition in an ALS/FTD subject relative to a subject which does not receive the composition.

The compounds of the present invention and their salts and solvates, thereof, may be employed alone or in combination with other therapeutic agents for the treatment of the diseases or conditions associated with the repeat RNA [G₄C₂^{exp}] in intron 1 of chromosome 9 open reading frame 72 (C9orf72).

The compounds of the invention and their pharmaceutical compositions are capable of functioning prophylactically and/or therapeutically and include administration to the host/patient of one or more of the subject compositions. If it is administered prior to clinical manifestation of the unwanted condition (e.g., disease or other unwanted state of the host animal/patient) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The compounds of the invention and their pharmaceutical compositions are capable of prophylactic and/or therapeutic treatments. If a compound or pharmaceutical composition is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). As used herein, the term “treating” or “treatment” includes reversing, reducing, or arresting the symptoms, clinical signs, and underlying pathology of a condition in manner to improve or stabilize a subject's condition.

The compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated, e.g., at a reasonable benefit/risk ratio applicable to any medical treatment.

### Administration

Compounds of the invention and their pharmaceutical compositions prepared as described herein can be administered according to the methods described herein through use of various forms, depending on the disorder to be treated and the age, condition, and body weight of the patient, as is well known in the art. As is consistent, recommended and required by medical authorities and the governmental registration authority for pharmaceuticals, administration is ultimately provided under the guidance and prescription of an attending physician whose wisdom, experience and knowledge control patient treatment.

For example, where the compounds are to be administered orally, they may be formulated as tablets, capsules, granules, powders, or syrups; or for parenteral administration, they may be formulated as injections (intravenous, intramuscular, subcutaneous or intrathecal), drop infusion preparations, or suppositories. For application by the ophthalmic mucous membrane route or other similar transmucosal route, they may be formulated as drops or ointments.

These formulations for administration orally or by a transmucosal route can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. Although the dosage will vary depending on the symptoms, age and body weight of the patient, the gender of the patient, the nature and severity of the disorder to be treated or prevented, the route of administration and the form of the drug, in general, a daily dosage of from 0.0001 to 2000 mg, preferably 0.001 to 1000 mg, more preferably 0.001 to 500 mg, especially more preferably 0.001 to 250 mg, most preferably 0.001 to 150 mg of the compound is recommended for an adult human patient, and this may be administered in a single dose or in divided doses. Alternatively, a daily dose can be given according to body weight such as 1 nanogram/kg (ng/kg) to 200 mg/kg, preferably 10 ng/kg to 100 mg/kg, more preferably 10 ng/kg to 10 mg/kg, most preferably 10 ng/kg to 1 mg/kg. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those excipients, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Pharmaceutical Compositions Incorporating ALS Compounds of Formula II

The pharmaceutical compositions of the invention incorporate embodiments of ALS Compounds of Formula II, preferably ALS compounds of Formulas II and III, useful for methods of the invention and a pharmaceutically acceptable carrier. The compositions and their pharmaceutical compositions can be administered orally, topically, parenterally, by inhalation or spray or rectally in dosage unit formulations. The term parenteral is described in detail below. The nature of the pharmaceutical carrier and the dose of these ALS Compounds depend upon the route of administration chosen, the effective dose for such a route and the wisdom and experience of the attending physician.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:
(1) fillers or extenders, such as starches, cyclodextrins, lactose, sucrose, glucose, mannitol, and/or silicic acid;
(2) binders, such as, for example, carboxymethylcellulose, alginates, gelatin, polyvinyl pyrrolidone, sucrose, and/or acacia;
(3) humectants, such as glycerol;
(4) disintegrating agents, such as agar-agar, calcium carbonate, potato or tapioca starch, alginic acid, certain silicates, and sodium carbonate;
(5) solution retarding agents, such as paraffin;
(6) absorption accelerators, such as quaternary ammonium compounds;
(7) wetting agents, such as, for example, acetyl alcohol and glycerol monostearate; (8) absorbents, such as kaolin and bentonite clay;
(9) lubricants, such a talc, calcium stearate, magnesium stearate, solid polyethylene glycols, sodium lauryl sulfate, and mixtures thereof; and
(10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound useful for application of methods of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound useful for practice of methods of the invention, it is desirable to slow the absorption of the compound from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically,” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) useful for application of the methods of the invention may be administered to humans and other animals for therapy by any suitable route of administration, including orally, nasally, as by, for example, a spray, rectally, intravaginally, parenterally, intracistemally, and topically, as by powders, ointments or drops, including buccally and sublingually.

Regardless of the route of administration selected, the compound(s) useful for application of methods of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) useful for application of methods of the invention in the pharmaceutical compositions of this invention may be varied so as to obtain an amount of the active ingredient which is effective to achieve the desired therapeutic response for a particular patient, composition, and mode of administration, without being toxic to the patient.

The concentration of a compound useful for application of methods of the invention in a pharmaceutically acceptable mixture will vary depending on several factors, including the dosage of the compound to be administered, the pharmacokinetic characteristics of the compound(s) employed, and the route of administration.

In general, the compositions useful for application of methods of this invention may be provided in an aqueous solution containing about 0.1-10% w/v of a compound disclosed herein, among other substances, for parenteral administration. Typical dose ranges are those given above and may preferably be from about 0.001 to about 500 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

### Experimental Examples

### Materials and Methods

### Quantification & Statistical Analysis

All quantification and statistical analyses (completed in GraphPad Prism version 8) were completed as described in the figure legends and in the methods. In brief, the statistical analysis for all experiments completed in c9ALS/FTD patient-derived cells accounted for repeated measurements of the same patient cell line using a Repeated Measures One- or Two-way ANOVA. Tukey's multiple comparison test was used to compare multiple samples as indicated in figure legends. For studies completed in vitro or in HEK293T cells, statistical significance was determined by using a One-way ANOVA or t-test as indicated. For all panels where statistical significance is indicated, * P<0.05, ** P<0.01, *** P<0.001, and **** P<0.0001. Bar graphs display individual data points and reported as the mean±SD. All compound-treated samples were normalized to vehicle unless otherwise noted. The DMSO concentration for vehicle-treated samples was always <0.1%.

### Biochemical & Biophysical Methods

General. RNAs and 5′-biotinylated oligonucleotides were purchased from Dharmacon (GE Healthcare) and deprotected according to the vendor's recommended procedure. After deprotection, the RNAs were desalted using PD-10 columns (GE Healthcare) and the concentrations were determined by UV/VIS spectrometry by measuring the absorbance at 260 nm at 90° C. using a Beckman Coulter DU 800 spectrophotometer and the corresponding extinction coefficient. DNA oligonucleotides were obtained from Integrated DNA Technologies (IDT) and used without further purification. Secondary structures were obtained by using the RNAstructure software (51, 52).

TR-FRET hnRNP H Binding Assay. TR-FRET assays were performed as previously described (12). The mixture was incubated for 15 min at room temperature and then hnRNP H1-His₆ was added. After an additional 15 min incubation, streptavidin-XL665 (HTRF, Cisbio Bioassays) and anti-His₆-Tb (HTRF, Cisbio Bioassays) were added to a final concentration of 40 nM and 0.44 ng/μL, respectively. After incubating for 1 h, TR-FRET was measured as previously described (12).

Biolayer Interferometry (BLI). BLI studies were performed similarly as previously described (12) using an Octet RED96. Briefly, 5′-biotinylated nucleic acids were folded by heating at 95° C. for 4 min in 10 mM sodium phosphate buffer, pH 7, followed by slowly cooling to room temperature. Then, to the folded RNA were added the following components: 1.386 mL 1×DPBS (21-031-CV, Corning), 180 μL freshly prepared 1% (w/v) BSA, 36 μL 1% (v/v) Tween-20, and 18 μL DMSO (1% final concentration) in a final volume of 1.8 mL. The final concentration of RNA was 0.5 μM. Separately, serial dilutions of the compound of interest were prepared in the same buffer.

Streptavidin sensors (SA, #18-5019, ForteBio) were equilibrated with buffer for 10 min at room temperature using 96-well black microplate (Greiner; Catalog #: 655209). The following time intervals were used during data acquisition (30° C. with shaking at 1000 rpm): baseline step, 180 s; loading of RNA, 900 s; washing, 180 s; association of compound, 1800 s; and dissociation of compound, 1800 s. The resulting curves were analyzed and processed using Octet Data Analysis software version 9.1 by subtracting the response of the sensors recorded upon incubation with solutions containing compound and no RNA (parallel reference). The data were then fitted assuming reversible binding using the entire time interval for association and dissociation using either 1:1 or the 2:1 heterogeneous binding curve fitting model. The reported K_{d,n}, k_{on,n} and k_{off,n} values represent the average of two independent experiments.

Preparation of MNR Samples. RNA constructs were purchased from GE Dharmacon, Inc. After deprotection and desalting, RNAs were dissolved in buffer comprising 10 mM sodium phosphates and 100 mM LiCl at pH 7.0 and reannealed by heating to 95° C. for 4 min followed by cooling to room temperature. Solvent consisted of H₂O, to which 20 μL of D₂O was added to provide a lock signal. Final concentrations of RNA were 100 μM for 1D NMR spectroscopy and 400 μM of RNA for 2D NMR spectroscopy. Dimeric derivatives 2 and 3 were dissolved in DMSO-d₆ to 10 mM concentrations. NMR samples did not contain more than 3% of DMSO-d₆ by volume.

NMR Spectroscopy. NMR spectra were acquired on samples in Shigemi tubes using Bruker Avance 600 and 700 MHz spectrometers equipped with cryoprobes. 1D spectra were recorded on free form RNA, to which 2 and 3 was added to final compound/RNA molar ratios of 0.5 and 1.0. 2D spectra were acquired with 400 ms mixing times on samples containing 3 at 1.0 compound/RNA molar ratio. In 1D and 2D NMR spectra, WATERGATE and excitation pulse sequences were used to suppress the water signal, respectively (53-55). Proton chemical shifts were referenced internally to 4,4-dimethyl-4-silapentane-1-sulfonic acid (DSS, 0.1 mM). 1D spectra were processed using MestreNOVA software. 2D spectra were processed using NMRPipe and assigned using SPARKY (56).

Topoisomerase Assay. Topoisomerase inhibition was assessed in vitro using the TopoGen Human Topoisomerase II Assay Kit per manufacturer's recommended protocol. Samples were run on a 1% agarose gel post-stained with ethidium bromide. Topoisomerase inhibition was quantified as a percentage of linearized DNA compared to the positive control compound VP-16 supplied.

RIBOTAC Cleavage of r(G₄C₂)₄ in vitro: Gel Analysis. A model of r(G₄C₂)^{exp}, r(G₄C₂)₈ was radioactively labeled at its 5′ end and purified on a denaturing 15% polyacrylamide gel as previously described (12, 57). Then, 25 nM of labeled RNA was folded by heating at 95° C. for 5 min in 1× Rnase L buffer (25 mM Tris-HCl, pH7.4, and 100 mM NaCl) and allowed to slowly cool to room temperature. To the folded RNA was added 2-Mercaptoethanol (7 mM final concentration), ATP (50 □M final concentration), and MgCl₂ (10 mM final concentration). Compound was added, and the samples were incubated for 15 min at room temperature. Next, Rnase L was added to a final concentration of 25 or 50 nM, and the samples were incubated at 37° C. (1 h or overnight). Fragments were separated on a denaturing 15% polyacrylamide gel, imaged by phosphorimaging with a Typhoon FLA 9500, and quantified with BioRad's QuantityOne software. All studies were performed in duplicate.

RIBOTAC Cleavage of r(G₄C₂)₈ in vitro: FRET Assay. A FRET assay was developed to monitor RIBOTAC-induced cleavage of r(G₄C₂)₈ by Rnase L. This assay uses r(G₄C₂)₈ labeled with Cy5 and Cy3, at the 5′ and 3′ end, respectively (Dharmacon). Samples were prepared as described in “RIBOTAC Cleavage of r(G₄C₂)₄ in vitro: Gel Analysis”. After addition of Rnase L, the samples were plated into a 384-well plate and incubated at 37° C. (1 h or overnight). Fluorescence was measured on a Molecular Devices SpectraMax M5 plate reader (Cy3, Ex.: 554 nm; Em.: 568 nm; Cy5, Ex.: 649 nm, Em.: 666 nm; FRET, Ex.: 554 nm, Em.: 666 nm). All studies were performed in duplicate.

In vitro Chemical Cross-linking and Isolation by Pull-Down (Chem-CLIP) and Competitive Chemical Cross-linking and Isolation by Pull-Down (C-Chem-CLIP): Approximately 10,000 counts of ³²P 5′-end labeled r(G₄C₂)₈ was folded in 1×Folding Buffer at 95° C. for 1 min. After cooling to room temperature, the Chem-CLIP probe at the desired concentrations (5, 10, 100, 500, 1000, 5000, 10000 nM) was added, and the samples were incubated at 37° C. for 18 h. For C-Chem-CLIP studies, the parent compound (10, 100, 500, 1000, 5000, 10000 nM) was incubated with RNA for 1 h before addition of the Chem-CLIP probe (500 nM).

Cross-linked RNAs were clicked to biotin disulfate azide (10 μL of a 5 mM stock solution in DMSO) by incubating in a solution containing 25 mM HEPES (pH 7.1), 250 mM sodium ascorbate, 10 mM CuSO₄, and 50 mM THPTA (tris-hydroxypropyltriazolylmethylamine) for 6 h at 37° C. A 300 μL of streptavidin-agarose beads (Sigma-Aldrich) was washed three times and then resuspended in 1×PBS. An aliquot (20 μL) of the slurry was added to each sample, which was incubated for 1 h at room temperature. The samples were centrifuged and the supernatant containing unbound RNA was transferred to an unused tube. The beads were then washed three times with 1×PBS supplemented with 0.10% (v/v) Tween-20 and centrifuged, with each wash supernatant being added to the tube containing unbound RNA. The amounts of radioactivity in the supernatant and associated with the beads were quantified with a Beckman Coulter LS6500 Liquid Scintillation Counter.

### Cellular Methods

Cell Culture. HEK293T cells (CRL-3216) were acquired from ATCC. LCLs were acquired from the Coriell Institute and iPSCs were generated through Answer ALS and obtained from the Cedars Sinai iPSC core. Please see Table 5 for a summary of cell line demographics and the studies in which they were used.

HEK293T cells. HEK293T cells were maintained in Dulbecco's Modified Eagle Medium (DMEM; Corning) supplemented with 10% fetal bovine serum (FBS; Sigma Aldrich), 1% penicillin-streptomycin (P/S; Corning) and 1% glutaGRO supplement (Corning) at 37° C. and 5% CO₂. HEK293T cells were batch transfected at ˜80% confluency in 100 mm diameter dishes and plated into appropriate experimental plates (96- or 384-well) and treated with compound for 24-48 h, as indicated.

Lymphoblastoid cell lines (LCLs). LCLs, both patient-derived and from healthy donors, were maintained in RPMI supplemented with 10% FBS and 1% PS (growth medium).

iPSCs and iPSCs-derived motor neuron cells. C9orf72 ALS/FTD and healthy iPSCs were maintained in the Matrigel (356234, Corning) coated plates with mTeSR™1 feeder-free medium (STEMCELL Technologies; Catalog #85850,) or according to manufacturer's instructions.

iPSCs were treated for 4 days in Matrigel (356234, Corning) coated plates with mTeSR™1 feeder-free medium (STEMCELL Technologies; Catalog #85850). Cells were treated in 6-well plates on day 1 and 3 with compounds diluted in 0.1% final DMSO.

iPSCs were differentiated into spinal neuron cells using a previously the previously described direct induced motor neuron (diMNs) differentiation protocol (58) with modifications. Briefly, iPSCs were plated into Matrigel-coated 100 mm dishes (30-40% confluence). Neuroepithelial induction was performed by replacing the iPSC maintenance medium with Stage 1 medium [47.5% IMDM (Iscove's Modified Dulbecco's Medium) medium, 47.5% F12 medium, 1% NEAA (Non-Essential Amino Acid) (Life Technologies), 2% B27 (Invitrogen), 1% N2, 1% PSA (Penicillin-Streptomycin-Amphotericin), 0.2 μM LDN193189 (Stemgent), 10 μM SB431542 (STEMCELL Technologies) and 3 μM CHIR99021 (Sigma-Aldrich)]. After daily medium changes for 6 days, cells were detached with Accutase and seeded into 6-well plates (1.5×10⁶ cells/well) in 3 mL Stage 2 medium. Cells were maintained in Stage 2 medium [Stage 1 medium supplemented with 0.1 μM All-trans RA (Sigma-Aldrich) and 1 μM *SAG (Cayman Chemicals)], with daily changes, through Day 11. At Day 12, the cell medium was switched to full Stage 3 medium [47.5% IMDM medium, 47.5% F12 medium, 1% NEAA, 2% B27, 1% N2, 1% PSA, 0.1 μM Compound E (Millipore; Catalog #: 565790), 2.5 μM DAPT (Sigma-Aldrich), 0.1 μM db-cAMP (Millipore), 0.5 μM All-trans RA, 0.1 μM SAG, 20 ng/mL ascorbic acid, 10 ng/mL BDNF (STEMCELL Technologies), and 10 ng/mL GDNF (STEMCELL Technologies)], which was replaced with fresh medium every other day.

Spinal neurons were treated starting at day 15 of differentiation. Cells were treated in Matrigel coated 6-well plates with stage 3 differentiation medium (see above). Cells were treated every 3-4 days until day 32. Compounds were diluted in 0.10% DMSO. On day 32, cells were harvested for analysis.

Cell Viability. Lymphoblastoid cells were seeded in 96-well plate (˜10⁴ cells/well) overnight and then treated with compounds for 48 h. The cell viability was measured by CellTiter-Fluor™ Cell Viability Assay per the manufacturer's protocol. For iPSCs, cells (diluted 1:4 after harvesting from a 100 mm dish) were seeded in Matrigel-coated 24-well plate. After 24 h, the compounds were added for 48 h and cell viability was measured by AlamarBlue™ Cell Viability Reagent (DAL1025, Thermo Fisher Scientific) per the manufacturer's protocol.

For iPSNs, cells (diluted 1:4 after harvesting from a 100 mm dish) were differentiated until Day 6 in Matrigel-coated 100 mm diameter dishes. Cells were then transferred to 96-well Matrigel-coated plates (˜10,000 cells/well) and differentiated until Day 18 or Day 32. Fresh medium containing compound was added starting on Day 15 and continuing every three days until the desired day was reached. Fresh medium was then added to the cells with AlamarBlue Cell Viability Reagent, and viability was measured per the manufacturer's protocol.

Cellular Proliferation. Patient-derived iPSCs (diluted 1:4 after harvesting from a 100 mm diameter dish) were seeded in Matrigel-coated 96-well plates (˜10,000 cells/well). The cells were treated for 48 h and 96 h, and cell proliferation was measured by Click-It EdU Cell Viability Reagent (C10337, Thermo Fisher Scientific) per the manufacturer's protocol. Patient-derived iPSNs (diluted 1:4 after harvesting from a 100 mm diameter dish) were seeded in Matrigel-coated 100 mm diameter dishes and differentiated until Day 6. Cells were then transferred to Matrigel-coated 96-well plates (˜10,000 cells/well) and differentiated until Day 15. Fresh medium containing compound was added starting on Day 15 and then every three days until the desired day was reached (either Day 18 or Day 32). Cellular proliferation was measured by Click-It EdU Cell Viability Reagent per the manufacturer's protocol.

RAN Translation Assay. Inhibition of RAN translation in HEK239T cells was completed as previously described (12). Briefly, HEK293T cells (6×10⁶ cells) were seeded in growth medium and incubated at 37° C. overnight. Cells were co-transfected with 2 μg of a plasmid encoding r(G₄C₂)₆₆-No ATG-NanoLuciferase and 1 μg SV40-Firefly luciferase plasmid with Lipofectamine 3000 (Life Technologies) per the manufacturer's protocols. Following transfection, the cells (˜8000/well) were plated in a clear bottom 384-well plate and incubated at 37° C. for 2 h. Compounds were added in growth medium such that the final concentration of DMSO was less than 1%. The G₄C₂-LNA and control-LNA (Table 3) were transfected with RNAiMax at a final concentration of 100 nM following transfection with RNAiMax (Life Technologies) per the manufacturer's recommended protocol. RAN and canonical translation was measured following 24 h treatment using a Dual-Luciferase® Reporter Assay System (Promega) following manufacturer's protocol. Experiments were performed as biological triplicates (n=3). Luminescence from Nano-Luciferase activity (RAN translation) was normalized to luminescence from firefly luciferase activity. This ratio for vehicle-treated samples was then set to 1 and the ratios for treated samples normalized accordingly.

Measuring RNA Levels in HEK Cells. HEK293T cells were seeded at 2×10⁶ in 100 mm dish and incubated at 37° C. overnight. Cells were transfected with 2.5 μg of the (G₄C₂)₆₆-No ATG-GFP plasmid with Lipofectamine 3000 (Life Technologies) per the manufacturer's protocols. After 5 h, the cells were collected and split into 24-well plate with 5×10⁴ cells per well. After 2 h, the compounds were added and incubated for another 24 h.

Total RNA was extracted using a Quick-RNA Miniprep Kit (Zymo Research) per the manufacturer's protocol. Approximately 1 μg of total RNA, as determined by Nanodrop, was used for reverse transcription using qScript Kit (Quantbio) per the manufacturer's protocol. RT-qPCR was performed on a 7900HT Fast Real Time PCR System (Applied Biosystem) using Power SYBR Green Master Mix (Applied Biosystems). Expression levels of mRNAs were normalized to β-actin. See Table 2 for a list of primers.

Measuring Levels of C9orf72 and C9orf72 Variants by RT-qPCR. LCLs were seeded in 6-well plate (˜10⁶ cells in 2 mL of growth medium) and incubated overnight at 37° C. Cells were then treated with various concentrations of compound for 24 h or with G₄C₂-ASO and Control-ASO (100 nM), delivered by transfection with Lipofectamine RNAiMax. RNA was extracted as described above and the expression of C9orf72 variants was determined by RT-qPCR. See Table 2 for a list of primers. For iPSC and iPSC-derived motor neuron cell line, the cells were seeded into Matrigel-coated 6-well plate to ˜60% confluence before treated with 3 μM C9orf72-ASO or Control-ASO and various concentrations of compound for 1 to 2 weeks. For compound treatment well, medium change was performed with fresh compound every 3-4 days. RNA was extracted as described above and the expression of C9orf72 variants was determined by RT-qPCR. See Table 2 for a list of primers.

Measuring Poly(GP) Levels by Electrochemiluminescence. LCLs were seeded in T-25 flash at 400,000 cells/mL in 5 mL of growth medium. After overnight incubation, the cells were treated with compound or ASO for 4 days at 37° C. After the treatment period, the cells were pelleted, and total protein was extracted by incubating with CoIP2 Buffer (50 mM Tris-HCL, pH 7.4, 300 mM NaCl, 5 mM EDTA, 1% (v/v) Triton-X 100, 2% (w/v) sodium dodecyl sulfate, and 0.01% (v/v) protease and phosphatase inhibitors) on ice for 5 min and then sonication (3 s intervals at 35% power for ˜40 s). Detergent was then removed using Pierce™ Detergent Removal Spin Columns following manufacturer's protocol. Protein concentration was measured by Pierce™ BCA Protein Assay Kit.

Levels of poly(GP) were measured using Meso Scale Discovery MSD Gold 96-well Streptavidin SECTOR plates. Both capture and detection antibodies were generated using an anti-poly(GP) antibody kindly provided by the Target ALS Foundation. To serve as the capture antibody, the poly(GP) antibody was conjugated to biotin using EZ-Link™ Sulfo-NHS-LC-Biotin (Thermo Fisher) as per the manufacturer's protocol. To serve as the detection antibody, the poly(GP) antibody was sulfo-tagged using MSD GOLD SULFO-TAG NHS-Ester (Meso Scale Discovery) as per the manufacturer's protocol. The conjugated antibodies were desalted with 40K Zeba™ Spin Desalting Columns (Thermo Fisher Scientific), and their concentrations were determined by Pierce™ BCA Protein Assay Kit.

Briefly, the plates were incubated with 2 μg/ml of anti-poly(GP)-biotin antibody at 4° C. overnight. The wells were then washed three times with 1×TBST [1×Tris-buffered saline (TBS) containing 0.1% (v/v) Tween-20] and blocked with 3% BSA in 1×TBST for 1 h at room temperature (with shaking). The blocking solution was removed, and the wells were washed 3 times with 150 μL of 1×TBST. Next, 80 □g of cell lysate or 100 □g of tissue lysate from in vitro and in vivo studies, respectively, were added to each well, and the samples were incubated at room temperature for 2 h with shaking. [No lysate was added to three wells to serve as background controls.] The wells were then washed with 150 μL of 1×TBST three times. The anti-poly(GP)-SULFO-Tag antibody (MSD; 4 μg/mL) was added in 3% BSA in 1×TBST, and the plate was incubated for 1 h at room temperature (with shaking). After washing with 150 μL of 1×TBST three times, 1×MSD GOLD Read Buffer was added to each well, and electrochemiluminescence was measured using a SECTOR Imager (MSD).

The average background signal from wells lacking lysate was subtracted from vehicle- and compound-treated samples. The signal from vehicle treated samples was set to 1 and compound-treated signals were normalized accordingly.

Measuring C9ORF72 Expression Level by Western Blotting. Samples analyzed by Western blot were prepared as described in “Measuring poly(GP) Levels by Electrochemiluminescence”. Approximately 20 μg of total protein was separated on a 10% SDS-polyacrylamide gel, and then transferred to a PVDF membrane. The membrane was washed with 1×TBS and then blocked in 5% milk in 1×TBST for 1 h at room temperature. After washing with 1×TBST, C9orf72 antibody (GeneTex, GTX119776; 1:3000 dilution) was added in 1×TBST containing 5% milk overnight at 4° C. The membrane was washed with 1×TBST and incubated with 1:2000 anti-mouse IgG horseradish-peroxidase secondary antibody conjugate (Cell Signaling Technology) in 1×TBST containing 5% (w/v) milk for 1 h at room temperature. The membrane was washed with 1×TBST, and protein expression was quantified using SuperSignal West Pico Chemiluminescent Substrate (Pierce Biotechnology) per the manufacturer's protocol.

To quantify β-actin expression, the membrane was stripped using 1× Stripping Buffer (200 mM glycine, pH 2.2 and 0.1% SDS) followed by washing in 1×TBST. The membrane was blocked and probed for p-actin similarly using 1:5000 β-actin primary antibody (Cell Signaling Technology) in 1×TBST containing 5% milk at room temperature for 1 h. The membrane was washed with 1×TBST and incubated with 1:10,000 anti-rabbit IgG horseradish-peroxidase secondary antibody conjugate (Cell Signaling Technology) in 1×TBST containing 5% (w/v) milk for 1 h at room temperature. β-actin protein expression was quantified using SuperSignal West Pico Chemiluminescent Substrate (Pierce Biotechnology) per the manufacturer's protocol. manufacturer's protocol.

GAPDH and β-tubulin expression were quantified following the same protocol as for β-actin expression, however, using; (i) a 1:5000 dilution of GAPDH primary antibody (Abcam, ab8245), followed by a 1:10,000 dilution of anti-mouse IgG horseradish-peroxidase secondary antibody conjugate (Cell Signaling Technology, 7076S) or (ii) a 1:5000 dilution of β-tubulin primary antibody (Abcam, ab52623) followed by a 1:10,000 dilution of anti-rabbit IgG horseradish-peroxidase secondary antibody conjugate (Cell Signaling Technology, 7074S). Protein expression for both proteins was quantified using SuperSignal West Pico Plus Chemiluminescence Substrate (Pierce Biotechnology) per the manufacturer's recommended protocol.

Imaging RNA Foci in LCLs. RNA foci containing r(G₄C₂)^{exp} were imaged using RNA fluorescence in situ hybridization (FISH) as previously described (13). Briefly, ALS patient-derived LCLs were treated with compound or ASO as described in “Measuring poly(GP) Levels by Electrochemiluminescence”. The cells were harvested, pelleted, and washed with 1×PBS. LCLs were then fixed in 2% paraformaldehyde in 1×PBS for 15 min at room temperature and then washed three times with 1×PBS. The fixed cells were then centrifuged, washed twice with 1×PBS, and seeded into poly lysine coated, glass bottom 96-well plates (˜5×10⁵ cells/well). To ensure the cells adhered to the plate, they were incubated at 37° C. for >2 h. The cells were then incubated with 70% ethanol overnight at 37° C. The next day, LCLs were washed with 1×PBS for 15 min at room temperature, and then with 1×PBS containing 0.1% (v/v) Triton X-100 for 5 min at room temperature. The cells were then washed with 40% formamide in 2×SSC (saline sodium citrate) for 15 min at room temperature followed by addition of the FISH probe [5 ng/μL 5′-Cy3-d(G₂C₄)₄] in 2×SSC containing 40% formamide, 2 μg/mL BSA, 330 ng/mL yeast tRNA, and 2 mM vanadyl complex. The samples were incubated at 37° C. for ˜24 h and then washed three times with 2×SSC (15 min, room temperature each) and then 1×PBS three times (15 min, room temperature each). Nuclei were stained with 1 μg/mL DAPI in 1×PBS for 10 min, and the cells were washed with 1×PBS. Cells were imaged in fresh 1×PBS using an Olympus Fluoview 1000 confocal microscope. Quantification was performed in triplicate with ˜200 nuclei counted per sample. Average number of foci per nuclei was assessed for each sample and reported. Statistical analysis was measured using an unpaired t-test with Welch's correction.

Target profiling by ASO-Bind-Map. LCLs were first treated with compounds overnight at 37° C. and then transfected with G₄C₂-ASO (100 nM; Table 3). The cells were then incubated for another 24 h at 37° C. followed by extraction of total RNA as described above. The levels of various fragments were then measured by RT-qPCR as described above.

Cellular Chem-CLIP. For cellular Chem-CLIP studies, LCLs were grown in T-25 flasks while iPSCs were grown in 100 mm dishes. The cells were treated with Chem-CLIP probe (500 nM) overnight at 37° C. After removing the medium and washing with 1×PBS, fresh, ice-cold 1×PBS was added, and crosslinking was performed by UV irradiation at 365 nm for 10 min. The cells were collected, and total RNA was extracted with Qiagen micro RNEasy Kit per the manufacturer's protocol.

Dynabeads MyOne Streptavidin C1 beads (Thermo Fisher Scientific 65001) were pre-loaded with biotin disulfide azide (Bioconjugate Technologies LLC dba Click Chemistry Tools) by incubating (10 μL of a 5 mM stock solution in DMSO in 500 μL PBS) with beads at room temperature for 30 min before magnetic capture. The beads were washed with High Salt Wash Buffer: (10 mM Tris-HCl pH 7.0, 1 mM EDTA, 4 M NaCl, 0.2% Tween-20) followed by two washes with Nanopure water.

The click reaction between cross-linked RNAs and the Dynabeads displaying biotin disulfide azide was completed in a solution containing 25 mM HEPES, pH 7.1, 250 mM sodium ascorbate, 10 mM CuSO₄, and 50 mM THPTA for 6 h at 37° C. The beads were washed with High Salt Wash Buffer (10 mM Tris-HCl, pH 7.0, 1 mM EDTA, 4 M NaCl, and 0.2% (v/v) Tween-20) and then twice with Nanopure water. The pulled-down RNA was then cleaved from the beads using a 50 mM DTT in 1×PBS (with shaking; 30 min at 37° C.). The supernatants of different dishes were combined, and the RNA was purified with RNA Clean XP beads (Beckman Coulter). The levels of C9orf72 mRNA in the purified, pull-down fraction were then measured by RT-qPCR as described above.

Polysome Profiling. Polysome profiling studies were completed similarly to previously described methods (12, 30) Yang et al., 2015). Briefly, 100 mm dishes were plated with ˜3.5×10⁶ HEK293T cells and batch-transfected for 5 h with 2.5 μg (G₄C₂)₆₆-No ATG-GFP using Lipofectamine 3000 reagent according to manufacturer protocol. Then, cells were collected and plated overnight in 100 mm dishes with ˜2.5×10⁶ HEK293T cells and treated with vehicle (DMSO, 0.5% final concentration) or 3 (200 nM). Upon 24 h treatment, cells were treated with cycloheximide (CHX, 0.1 mg/mL) and incubated for 5 min at 37° C. The medium was removed, and cells were washed by 2×7.5 mL ice cold DPBS supplemented with 0.1 mg/mL CHX. Cells were then lysed with 250 μL of ice-cold Cell Lysis Buffer [10 mM NaCl, 10 mM MgCl₂, 10 mM Tris, pH 7.5, 1% Triton X-100, 1% sodium deoxycholate, 1 mM DTT supplemented with 0.1 mg/L cycloheximide, and 0.2 U/μL RNAsin (Promega)]. The lysate obtained from 3×100 mm dishes were combined (˜500 μL final volume) and transferred to an Eppendorf tube, passed through a 21G needle several times and centrifuged for 5 min at 13,200 rpm and 4° C. The supernatant was transferred to a new tube and stored at −80° C. until further use. Cell lysates were then loaded on 10-50% sucrose gradients (prepared in 20 mM HEPES, pH 7.4, 100 mM KCl, 5 mM MgCl₂, 3 mM DTT, and 0.1 mg/mL CHX) and separated at 40000 rpm for 2 h at 4° C. RNA was extracted from 200 μL aliquots from each fraction (total volume of 500 μL) by adding 400 μL RNA lysis buffer and 600 μL EtOH following the Quick RNA™ Mini-Prep (Zymo Research) manufacturer's protocol. cDNA was generated from 100 ng of RNA using a Qscript cDNA Synthesis Kit according to manufacturer's recommended procedure. The levels of (G₄C₂)₆₆-No ATG-GFP mRNA in each fraction were measured by qPCR as described above with primers specific for GFP (non-canonical translation), GAPDH, respectively (canonical translation) (Table 1). Data from vehicle- and compound-treated samples were normalized as follows: triplicate Cₜ values were averaged and ΔCₜ values were calculated; ΔΔCₜ values were afforded by comparing treated to untreated ΔCₜ values. Data were then normalized to the fraction with the lowest abundance of GFP.

RNA Immunoprecipitation. HEK293T cells were transfected with (G₄C₂)₆₆-No ATG-GFP plasmid as described above, split into in 6-well plates, grown to ˜70% confluency, and treated with 500 nM 7 diluted in growth media for 24 h. The cell was washed with 1×PBS, removed from the plate with Accutase (Innovative Cell Technologies, Inc.), and then washed with 1×PBS. Cells were then lysed in 100 μL of M-PER buffer supplemented with 80 U RNaseOUT Recombinant Ribonuclease Inhibitor (Invitrogen) and 1× Protease Inhibitor Cocktail III for Mammalian Cells (Research Products International Corp.) per manufacturer's recommendations. The protein samples were tumbled overnight at 4° C. with Dynabeads Protein A (Life Technologies) that were bound to either β-actin mouse primary antibody (Cell Signaling Technologies; 3700S) or Rnase L mouse primary antibody (Santa Cruz Biotechnology; sc-74405). After antibody incubation, the beads were washed three times with 1×PBS supplemented with 0.02% Tween-20 and total RNA was extracted from beads using a miRNeasy Mini Kit (Qiagen) according to manufacturer's instructions. RT-qPCR was completed as described above with primers listed in Table 2.

Relative RNA expression levels before and after pull-down were determined by the ΔΔCₜ method and normalized to β-actin as a housekeeping gene.

Rnase L Knockdown. HEK293T cells were seeded in growth medium in a 100 mm dish and incubated at 37° C. until ˜80% confluent. Cells were transfected with 2.5 □g (G₄C₂)₆₆-No-ATG-GFP for 5 h with Lipofectamine 3000 (Life Technologies) per the manufacturer's recommended protocol. Following transfection, the cells were plated in 6-well plates and incubated at 37° C. until ˜80% confluent. Compounds were added in growth medium and incubated for 24 h. Following compound treatment, the siRNA of interest (Table 3) were transfected with RNAiMax (Life Technologies) at a final concentration of 50 nM for 24 h per the manufacturer's recommended protocol. Total RNA was extracted using a Quick-RNA Miniprep Kit (Zymo Research) per the manufacturer's protocol. Intron 1 abundance was assessed by RT-qPCR as described above.

Transcriptome-wide Studies via RNA-seq. Patient-derived iPSCs were differentiated into motor neuron cells as described above. At day 21, the cell in Matrigel-coated 6-well plate was treated with 3 μM C9orf72-ASO, 3 μM Control-ASO and various concentrations of compounds. For the compound treatment, the medium was changed every 3 to 4 days with fresh compounds. After 2 weeks' treatment, total RNA was extracted with Qiagen Rneasy Mini Kit. The cleavage of target G₄C₂-containing C9orf72 variants was confirmed by RT-qPCR with primers listed in Table 2. The RNA samples were then subjected to RNA-seq analysis.

Nuclei Isolation and Super Resolution Structured Illumination Microscopy. iPSC-derived spinal neurons were generated according to the previously published diMNs protocol (37). On day 15 and 18 of differentiation, 5 QM G₄C₂ repeat-targeting ASO was added to media as previously described (59). For compound treatment, 50 nM 7 was added to media on days 15, 18, 21, 24, 27, and 30 of differentiation. On day 32 of differentiation, nuclei isolation was performed using the Nuclei Pure Prep Nuclei Isolation Kit (Sigma Aldrich) as previously described (68). Isolated nuclei were spun onto collagen coated slides and immunostained following standard protocols as previously described (68). Primary antibodies: 1:250 rat anti-Nup98 (Abcam), 1:500 chicken anti-NeuN (Millipore); secondary antibodies: goat anti-rabbit Alexa 488 (Invitrogen), goat anti-chicken Alexa 647 (Invitrogen). NeuN positive nuclei were imaged by super resolution structured illumination microscopy (SIM) using a Zeiss ELYRA S.1 as previously described (14). Next, 50 nuclei per iPSC line/treatment group were analyzed using the 3D suite plugin in FIJI. The average number of spots from the 50 nuclei for each cell line and treatment group was used for statistical analyses. Two-way ANOVA with Tukey's multiple comparison test was used to calculate statistical significance. Images are presented as maximum intensity projections. Quantification is presented as violin plots to represent the full variability of all nuclei analyzed.

### In Vivo Methods

Therapeutic Efficacy in a c9ALS/FD mouse model. All animal studies were completed as approved by the Scripps Florida Institutional Animal Care and Use Committee. A total of 24 mice (12+/+PWR500 [7 Male and 5 Female] and 12 WT [6 Male and 6 Female]) mice, age- and gender-matched and ranging in age from 18-22 weeks old were used in therapeutic efficacy studies (Table 6). Mice were anesthetized by intraperitoneal (i.p.) injection of 10 mg/mL Ketamine:1 mg/mL Xylazine solution. Intracerebroventricular (ICV) stereotactic injections of 10 μL of 7 (33 nmol) were administered into the right ventricle using the following coordinates: 0.2 mm posterior and 1.0 mm lateral to the right from the bregma and 3 mm deep. Mice were treated with 7 or vehicle consisting of 1% DMSO in Saline. Three weeks post-treatment, mice were euthanized, and tissue was harvested for study.

Measuring C9orf72 Variants by RT-qPCR. Postmortem brain tissue was harvested and sliced along the midline. The left hemisphere was frozen for RNA and protein analysis. The left hemisphere of each brain was homogenized in 300 μL ice cold Tris-EDTA buffer with 2× protease inhibitors. 150 μL of homogenized tissue was used for RNA extraction. 450 μL of triazol LS was added to the homogenized tissue and centrifuged for 15 min at 16,000 rpm. Following centrifugation, supernatant was collected and an equal volume (600 μL) of 100% ethanol was added and mixed thoroughly. RNA was purified using a Direct-zol RNA Kit (Zymo Research) per the manufacturer's protocol. RT-qPCR was performed as described above. Expression levels of mRNAs were normalized to mouse (3-actin. See Table 2 for a list of primers. Statistical analysis was measured using an unpaired t-test with Welch's correction.

Measuring Poly(GP) by MSD. Postmortem brain tissue was harvested and sliced sagittal at the midline. The left hemisphere was frozen for protein and RNA analysis. The left hemisphere of each brain was homogenized in 300 μL of ice-cold Tris-EDTA buffer with 2× protease inhibitors. 150 μL of homogenized tissue was mixed with 2×lysis buffer (50 mM Tris pH 7.4, 250 mM NaCl, 2% Triton X-100, 4% SDS, and 1× protease inhibitor). Brain lysates were sonicated on ice at 1 s on/off intervals at 30% for 15 s. Protein concentrations were measured by BCA assay (Pierce Biotechnology) and Poly(GP) was measured as described above. Statistical analysis was measured using an unpaired t-test with Welch's correction.

Immunohistochemistry. Tissue was excised postmortem, and brain tissue was sliced at the midline. The right hemisphere was stored in 10% neutral buffered formalin for 48 h. Tissue processing, embedding and sectioning were carried out by the Scripps Florida's Histology Core. Formalin-fixed tissue was placed on a paraffin processor (Sakura Tissue-Tek VIPS), embedded in paraffin, sectioned a 4 μm, and mounted on plus slides. The slides were optimized with the appropriate primary antibody dilutions (see below) on the Leica BOND-MAX platform followed by the Leica Refine Detection Kit containing the secondary polymer, DAB chromagen and counterstain. After dehydration in graded alcohols and cleared in xylene, the slides were cover slipped with a permanent mounting medium, Cytoseal 60 (Thermo Scientific). Images were taken in the right hemisphere of the cortex due north of the hippocampus with 5 images were taken per slice. Average number of aggregates or inclusions per cell was assessed for each sample and reported. Statistical analysis was measured using an unpaired t-test with Welch's correction. Immunohistochemical staining and analyses were completed blinded.

Antibodies. NeuN (1:2000, RRID: AB_177621 Millipore); Poly-GA (1:2000, MABN889, Millipore); Poly-GP (1:5000, ABN455, Millipore); Calbindin (1:5000, RRID: AB_476894, Millipore); ChAT (1:100, RRID: AB_262156, Millipore); TDP-43 (1:2000, RRID: AB_615042, Proteintech).

Fluorescence in situ Hybridization (FISH) with Immunofluorescence (IF). Tissues were excised, and brain tissue was sliced at the midline. The right hemisphere was fresh frozen with OCT in 2-methylbutane in liquid nitrogen, and 10 μm thick sections were prepared using a cryostat. The slides were then stained as previously described (36). Briefly, frozen sections were fixed in 4% paraformaldehyde in 1×DPBS for 20 min and incubated in ice-cold 70% ethanol for 30 min at 4° C. Sections were then incubated in 40% formamide in 2×SSC Buffer for 10 min at room temperature. Slides were blocked in 1× Hybridization Buffer (40% formamide, 2×SSC, 20 μg/mL BSA, 100 mg/mL dextran sulfate, 250 μg/mL tRNA, and 2 mM vanadyl sulfate) for 30 min at 55° C. Following blocking, slides were incubated with 200 ng/mL of denatured LNA probe (Table 3) in 1× Hybridization Buffer for 3 h at 55° C. Slides were then washed three times in 40% formamide in 2×SSC Buffer followed by one wash in 1×DPBS. Slides were co-stained with NeuN (Sigma: MAB377B) as follows. The slides were incubated for 15 min with 0.5% (v/v) Triton X-100 in 1×DPBS at 4° C. and then blocked with 2% goat serum diluted in 1×DPBS [blocking solution] for 1.5 h at 4° C. After blocking, the slides were incubated overnight at 4° C. with NeuN (1:500, MAB377B, Millipore) diluted in the blocking solution. After washing three times with 1×DPBS, the sample were incubated with donkey anti-goat conjugated to Alexa Fluor 488 (AbCam Inc), diluted 1:500 in 1×DPBS, for 1 h at room temperature. The slides were washed three with 1×DPBS and quenched with 0.25% Sudan Black B (Millipore) diluted in 70% ethanol. After washing three times with 1×DPBS, the slides were allowed to completely dry, mounted with mounting medium containing DAPI (Invitrogen) and imaged using a 60×objective. Average number of foci per nuclei was assessed for each sample and reported. Statistical analysis was measured using an unpaired t-test with Welch's correction.

### Computational Methods

Parameterization Small Molecule 1 (Compound 1), PEG and NH-modified PEG. All Amber force field parameters of 1 (FIG. 1 and below) were obtained from previous study (table below) (5). The force field parameters of the NH-modified PEG linker (NH-PEG; below) were prepared as previously described (6-8). The AMBER GAFF force field (9), was used to define the atom types while RESP charges were derived following the multimolecular RESP charge fitting protocol (Table 4) (10, 11). The molecules were optimized and the electrostatic potentials as a set of grid points were calculated at the HF level using the 6-31G* basis set, where Gaussian09 (12), was used to perform these calculations. These residues were used to build the dimer structure of 3, which consist of two 1 RNA-binding modules and NH-modified PEG linker (FIG. 15).

Binding Study. Previously, we determined the binding modes of 1 to a model of r(G₄C₂) repeats (5). The lowest energy binding modes were used to homology model two 1 molecules bound to 5′-GCCCGGGGCCCG-3′/5′-CGGGGCCGGGGC-3′, a duplex model of r(G₄C₂) repeats. This structure was used to build the bound states of 3. The structures were minimized to remove artifacts such as poor contacts and unrealistic bonds, arising from homology modeling.

Explicit Solvent Molecular Dynamics (MD) Simulation. Explicit water MD simulations were performed to find optimal bound conformations. The initial coordinates for MD simulations were extracted from the results of homology modeling, and 21 Na⁺ ions (13), were added to make the system neutral. TIP3P water molecules were added to the systems so that all the atoms of RNA and 3 were at least 8.0 Å away from the edge of the simulation box. Long-range electrostatic interactions were calculated using the Particle Mesh Ewald method (14). Temperature and the pressure were maintained through the simulations as 300 K and 1 bar using Langevin dynamics and Berendsen barostat. Three independent MD simulations for 500 ns with a time step of 1 fs were completed. The total of 1.5 μs combined MD trajectories were produced and used in cluster analysis.

Cluster Analysis and MM-PBSA Calculation. Cluster analysis was conducted to determine structure population using CPPTRAJ. CPPTRAJ groups similar conformations together in the a given trajectory file by Root-mean-square deviation (RMSD) analysis. The density-based scanning algorithm was used with RMSD cutoff distance of 1.3 Å to form a cluster. Cluster analysis revealed three stable binding conformations. MM-PBSA analyses were conducted on each cluster to determine the lowest binding free energy states. The MMPBSA.py module of AMBER16 was used and the results of relative binding free energies for are presented in Table 4. The binding conformations with the lowest binding energies were selected as the most stable binding conformations.

### Synthetic Methods

Abbreviations: Ac₂O, acetic anhydride; CDCl₃, chloroform-d; CD₃OD, methanol-d₄; Cs₂CO₃, Cesium carbonate; DIPEA, N,N-diisopropylethylamine; DCM, dichloromethane; DMF, N,N-dimethylformamide; DMSO, dimethyl sulfoxide; EDC, N-Ethyl-N′-(3-dimethylaminopropyl)carbodiimide hydrochloride; Et₃N, triethylamine; EtOAc, ethyl acetate; HCl, hydrochloric acid; H₂O, water; HOBt, 1-hydroxybenzotriazole; HPLC, high performance liquid chromatography; K₂CO₃, potassium carbonate; LiCl, lithium chloride; MALDI, matrix-assisted laser desorption/ionization; MeOH, methanol; NaH, sodium hydride; NaHCO₃, sodium bicarbonate; NaI, sodium iodide; NaOMe, sodium methoxide; Na₂SO₄, sodium sulfate; NMR, nuclear magnetic resonance; PEG, polyethylene glycol; TFA, trifluoroacetic acid; THF, tetrahydrofuran; TLC, thin layer chromatography.

General. Reagents and solvents were purchase from standard suppliers and used without further purification. Reactions were monitored by TLC. Spots were visualized with UV light or by phosphomolybdic acid or Ninhydrin staining. Products were purified by Isolera One flash chromatography system (Biotage) using pre-packed silica gel column (Agela Technologies) or by HPLC (Waters 2489 pump and 1525 detector) using a SunFire® Prep C18 OBD™ 5 μm column (19×150 mm) with the flow rate of 5 mL/min. Compound purity was analyzed by HPLC using a SunFire® C18 3.5 μm column (4.6×150 mm) with the flow rate of 1 mL/min. NMR spectra were measured by a 400 UltraShield™ (Bruker) (400 MHz for ¹H and 100 MHz for ¹³C) or Ascend™ 600 (Bruker) (600 MHz for ¹H and 150 MHz for ¹³C). Chemical shifts are expressed in ppm relative to trimethylsilane (TMS) for ¹H and residual solvent for ¹³C as internal standards. Coupling constant (J values) are reported in Hz. High resolution mass spectra were recorded on a 4800 Plus MALDI TOF/TOF Analyzer (Applied Biosystems) with α-cyano-4-hydroxycinnamic acid matrix and TOF/TOF Calibration Mixture (AB Sciex Pte. Ltd.) or an Agilent 1260 Infinity LC system coupled to an Agilent 6230 TOF (HR-ESI) equipped with a Poroshell 120 EC-C18 column (Agilent, 50 mm×4.6 mm, 2.7 μm).

### Synthetic Experimental Procedures

Intermediate compounds i3-i7 were synthesized according to literature procedures as follows: i3-i5 (15), i6 (16) and i7 (17). Derivative i14 is commercially available and was used as received. Syntheses of dimeric compounds 9-12 were completed similarly to derivative 2.

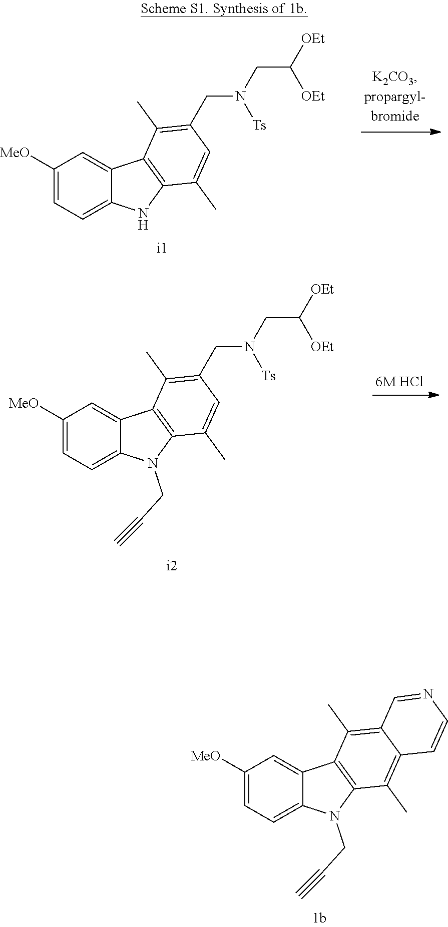 [SMILES: CCOC(CN(Cc1cc(C)c2[nH]c3ccc(OC)cc3c2c1C)S(=O)(=O)c1ccc(C)cc1)OCC ; C#CCn1c2ccc(OC)cc2c2c(C)c(CN(CC(OCC)OCC)S(=O)(=O)c3ccc(C)cc3)cc(C)c21 ; C#CCn1c2ccc(OC)cc2c2c(C)c3cnccc3c(C)c21]

### Compound i2

To a solution of i1 (18) (200 mg, 0.38 mmol) in anhydrous DMF (2 mL), was added NaH (60% oil suspension, 14 mg, 1.5 eq) at 0° C. The solution turned yellow immediately and the mixture was stirred for an additional 15 min. Afterwards, propargyl bromide (46 mg, 1 eq) was added dropwise and the mixture was left to warm to room temperature and stirred for an additional 4 h. The reaction mixture was diluted with ethyl acetate (EtOAc; 15 mL) and partitioned with water. The organic layer was washed with 5% LiCl and brine, dried over anhydrous Na₂SO₄, filtered, and concentrated under reduced pressure. The residue was purified by flash chromatography (20%-40% linear gradient in hexanes), affording i2 (180 mg, 84%) as a white solid.

¹H NMR (400 MHz, CDCl₃): δ=7.80-7.67 (m, 3H), 7.38 (d, J=8.8 Hz, 1H), 7.33-7.23 (m, 3H), 7.15 (dd, J=2.5, 8.8 Hz, 1H), 6.91 (s, 1H), 5.20 (d, J=2.5 Hz, 2H), 4.66 (s, 2H), 4.45 (t, J=5.4 Hz, 1H), 3.95 (s, 3H), 3.67-3.47 (m, 2H), 3.47-3.25 (m, 2H), 3.22 (d, J=5.4 Hz, 2H), 2.79 (s, 3H), 2.75 (s, 3H), 2.40 (s, 3H), 2.29 (t, J=2.5 Hz, 1H), 1.11 (t, J=7.0 Hz, 6H); ¹³C NMR (101 MHz, CDCl₃): δ=154.06, 143.19, 139.16, 137.49, 136.30, 131.85, 131.22, 129.58, 127.38, 124.81, 124.33, 123.18, 117.45, 113.42, 109.12, 107.41, 102.19, 79.33, 72.85, 63.26, 56.36, 50.81, 49.66, 34.84, 21.54, 19.57, 16.17, 15.39; HRMS (ESI) calculated for C₃₂H₃₉N₂O₅S [M+H]⁺: 563.2574. found: 563.2582.

Synthesis of 1b. Intermediate i2 (180 mg, 0.32 mmol) was dissolved in dioxane (20 mL) and 6 M HCl (5 mL) was added. The reaction mixture was heated to 110° C. for 3 h. Excess of HCl was removed under air stream, the resulting mixture was concentrated under reduced pressure and resuspended in MeOH. The product was purified by reversed phase flash chromatography (C18 column; gradient of 15% to 100% MeOH/water+0.10% TFA), yielding 1b (97 mg, 71%) as a yellow solid.

¹H NMR (400 MHz, DMSO-d₆): δ=9.77 (s, 1H), 8.35 (s, 2H), 7.62 (d, J=8.6 Hz, 1H), 7.51 (s, 1H), 7.22 (d, J=8.4 Hz, 1H), 5.37 (s, 2H), 3.82 (s, 3H), 3.52 (s, 1H), 2.99 (s, 3H), 2.95 (s, 3H); ¹³C NMR: (101 MHz, DMSO-d₆) δ: 154.57, 143.57, 143.25, 137.70, 134.85, 133.26, 128.13, 126.03, 122.11, 119.91, 119.58, 116.18, 110.59, 110.54, 107.93, 79.63, 76.51, 55.65, 35.67, 14.79, 12.96; HRMS (ESI) calculated for C₂₁H₁₉N₂O⁺ [M+H]⁺: 315.1492. found: 315.1498.

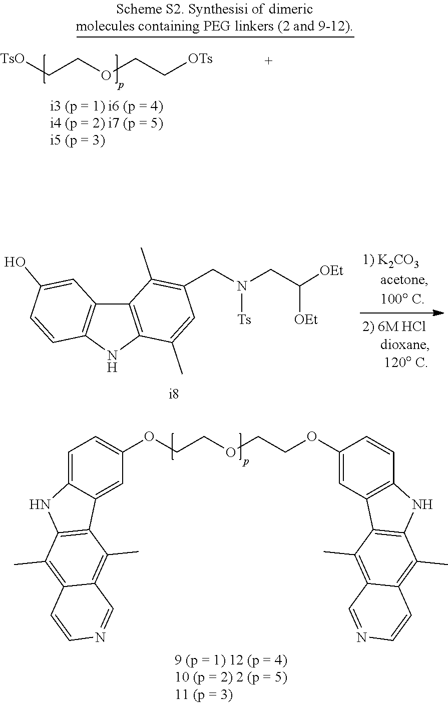

### Dimeric Compound 2

To a suspension of i8 (5) (80 mg, 0.156 mmol, 2.5 eq) in acetone (1.5 mL) was added i7 (0.062 mmol, 1.0 eq) and K₂CO₃ (0.156 mmol, 2.5 eq). The reaction mixture was heated to 100° C. and stirred for 12 hours before it was diluted with H₂O and EtOAc (10 mL each). The phases were separated, and the product was extracted with EtOAc (3×15 mL). The combined organic extracts were dried over Na₂SO₄, filtered and the solvent was removed in vacuo. The crude product was co-evaporated with silica and purified by flash chromatography (10-100% EtOAc linear gradient in hexanes) providing the O-alkylated dimer, which was subsequently dissolved in dioxane (1.5 mL), treated with HCl (6 M, 270 μL) and heated to 120° C. for 6 h. The solvent was evaporated, and the crude product was purified by HPLC (linear gradient 10-100% acetonitrile/MeOH=1:1, 0.1% TFA, 10 min) yielding 2 (18.2 mg, 38% over 2 steps). All the compound concentrations were assessed via UV absorption using the extinction coefficient of compound 1a measured at 300 nm in 100% DMSO (ε_{300 nm}=33572 mol·L⁻¹ cm⁻¹).

¹H NMR (700 MHz, CD₃OD): δ=8.88 (s, 1H), 7.91 (d, J=6.3 Hz, 1H), 7.64 (d, J=6.3 Hz, 1H), 6.63 (d, J=8.1 Hz, 1H), 6.41 (dd, J=2.3, 8.1 Hz, 1H), 6.36 (s, 1H), 3.82-3.75 (m, 10H), 3.72-3.67 (m, 2H), 2.13 (s, 6H); ¹³C NMR (176 MHz, CD₃OD): δ=152.51, 143.51, 141.26, 135.75, 132.80, 131.59, 126.21, 124.45, 121.24, 118.47, 118.40, 115.63, 110.41, 108.76, 106.65, 70.20, 70.16, 69.96, 69.50, 67.40, 13.04, 10.25; HRMS (ESI) calcd. for C₄₆H₅₁N₄O₇+[M+H]⁺: 771.3752. found: 771.3776.

### Compound 9

¹H NMR (400 MHz, DMSO-d₆): δ=11.71 (s, 1H), 9.70 (s, 1H), 8.31 (d, J=7.0 Hz, 1H), 8.15 (d, J=7.0 Hz, 1H), 7.61 (d, J=2.4 Hz, 1H), 7.46 (d, J=8.7 Hz, 1H), 7.23 (dd, J=2.4, 8.7 Hz, 1H), 4.34-4.24 (m, 2H), 4.00-3.89 (m, 2H), 2.89 (s, 3H), 2.53 (s, 3H); HRMS (ESI) calcd. for C₃₈H₃₅N₄O₃+[M+H]⁺: 595.2704. found: 595.2726.

### Compound 10

¹H NMR (400 MHz, CD₃OD): δ=8.88 (s, 1H), 7.81 (d, J=6.8 Hz, 1H), 7.47 (d, J=6.8 Hz, 1H), 6.67 (d, J=8.6 Hz, 1H), 6.52 (dd, J=2.3, 8.6 Hz, 1H), 6.35 (d, J=2.3 Hz, 1H), 4.00-3.79 (m, 6H), 2.23 (s, 3H), 2.00 (s, 3H); HRMS (ESI) calcd. for C₄₀H₃₉N₄O₄+[M+H]⁺: 639.2966. found: 639.2948.

### Compound 11

¹H NMR (400 MHz, CD₃OD): δ=9.04 (s, 1H), 7.94 (d, J=6.9 Hz, 1H), 7.70 (d, J=6.9 Hz, 1H), 6.57 (d, J=9.1 Hz, 1H), 6.23-6.20 (m, 2H), 3.91-3.83 (m, 2H), 3.81-3.74 (m, 2H), 3.58-3.53 (m, 4H), 2.30 (s, 3H), 2.18 (s, 3H); HRMS (ESI) calcd. for C₄₂H₄₃N₄O₅+[M+H]⁺: 683.3228. found: 683.3226.

### Compound 12

¹H NMR (400 MHz, CD₃OD): δ=9.02 (s, 1H), 7.94 (d, J=6.9 Hz, 1H), 7.75 (d, J=6.9 Hz, 1H), 6.71 (d, J=8.6 Hz, 1H), 6.51 (s, 1H), 6.46 (dd, J=2.3, 8.6 Hz, 1H), 3.81-3.74 (m, 4H), 3.74-3.68 (m, 4H), 3.67-3.58 (m, 2H), 2.30 (s, 3H), 2.21 (s, 3H); HRMS (ESI) calcd. for C₄₄H₄₇N₄O₆+[M+H]⁺: 727.3490. found: 727.3506.

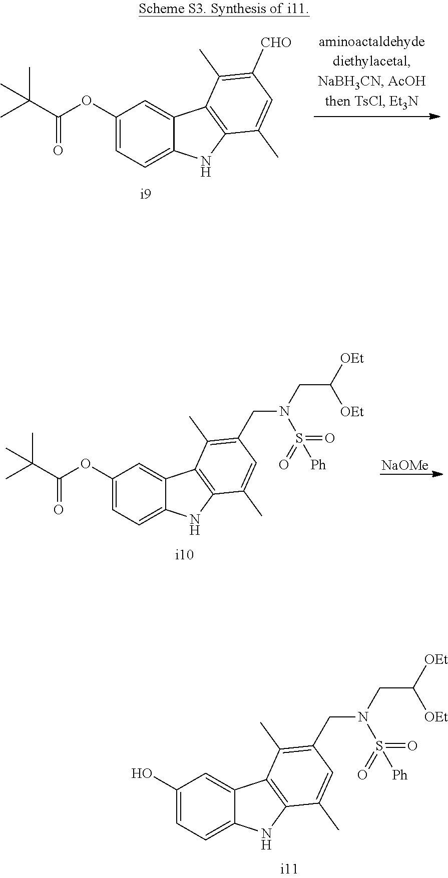

### Compound i10

A mixture of i9 (5) (1.9 g, 6 mmol), aminoacetaldehyde diethylacetal (1.1 mL, 7.5 mmol), acetic acid (601 μL, 10.5 mmol), and sodium cyanoborohydride (660 mg, 10.5 mmol) in ethanol (45 mL) was heated to 83° C. for 1 h. The reaction was quenched by the addition of a saturated NaHCO₃ solution. The product was extracted with DCM, washed with brine, and dried over Na₂SO₄. The solvent was evaporated, and the product was used for the next step without further purification. Tosylchloride (842 μL, 6.6 mmol) was added to a mixture of the crude product and Et₃N (1.7 mL, 12 mmol) in THF (14 mL) at 0° C. The reaction mixture was stirred at room temperature for 1.5 h. The solvent was evaporated, and the product was extracted with EtOAc. The combined organic extracts were washed with sat. NaHCO₃ and brine, dried over anhydrous Na₂SO₄ and the solvent was removed under reduced pressure.

The crude product was purified by silica gel column chromatography (Hexane:DCM:AcOEt=6:3:1, 1% Et₃N) to give i10 (2.5 g, 4.4 mmol, 73% for 2 steps) as a white solid.

¹H NMR (600 MHz, CDCl₃): δ=8.09 (s, 1H), 7.90-7.86 (2H), 7.76 (m, 1H), 7.56 (m, 1H), 7.52-7.47 (2H), 7.35 (d, J=8.6 Hz, 1H), 7.05 (dd, J=8.6, 2.2 Hz, 1H), 6.95 (s, 1H), 4.69 (s, 2H), 4.41 (t, J=5.5 Hz, 1H), 3.54-3.48 (2H), 3.30-3.24 (2H), 3.20 (d, J=5.5 Hz, 2H), 2.71 (s, 3H), 2.37 (s, 3H), 1.43 (s, 9H), 1.06 (t, J=7.0 Hz, 6H); ¹³C NMR (150 MHz, CDCl₃): δ=178.2, 144.2, 140.5, 139.3, 137.3, 132.2, 130.5, 129.0, 128.9, 127.2, 124.6, 124.0, 121.7, 118.7, 116.9, 115.0, 110.8, 101.8, 63.0, 50.4, 49.1, 39.1, 27.3, 16.4, 15.7, 15.2; HR-MS (MALDI): Calcd. for C₃₂H₄₀N₂NaO₆S [M+Na]⁺: 603.2499. found: 603.2506.

### Compound i11

To a solution of i10 (1.6 g, 2.8 mmol) in MeOH (15 mL) was added NaOMe (300 mg, 5.6 mmol). The mixture was stirred at room temperature for 3 h. The solvent was evaporated, water was added, and the product was extracted with EtOAc. The combined organic extracts were dried over Na₂SO₄, filtered and the solvent was removed under reduced pressure. The crude product was purified by silica gel column chromatography (20-40% EtOAc/Hexanes) to give i11 as a white solid (700 mg, 1.4 mmol, 50%).

¹H NMR (600 MHz, DMSO-d₆): δ=10.84 (m, 1H), 8.88 (m, 1H), 7.95-7.86 (2H), 7.72 (m, 1H), 7.68-7.62 (2H), 7.54 (m, 1H), 7.32 (t, J=8.3 Hz, 1H), 6.94-6.86 (2H), 4.52 (s, 2H), 4.05 (m, 1H), 3.42-3.25 (4H, including residual water peak), 3.14-3.00 (4H), 2.70 (m, 3H), 2.39 (m, 3H), 0.91 (t, J=7.0 Hz, 6H); ¹³C NMR (150 MHz, DMSO-d₆): δ=150.3, 139.5, 139.4, 134.2, 132.8, 129.5, 129.4, 128.2, 127.1, 123.9, 122.2, 121.0, 116.9, 114.1, 111.2, 107.3, 100.4, 62.0, 50.6, 48.9, 16.7, 15.3, 15.0; HR-MS (MALDI): Calcd. for C₂₇H₃₂N₂NaO₅S [M+Na]⁺: 519.1924. found: 519.1931.

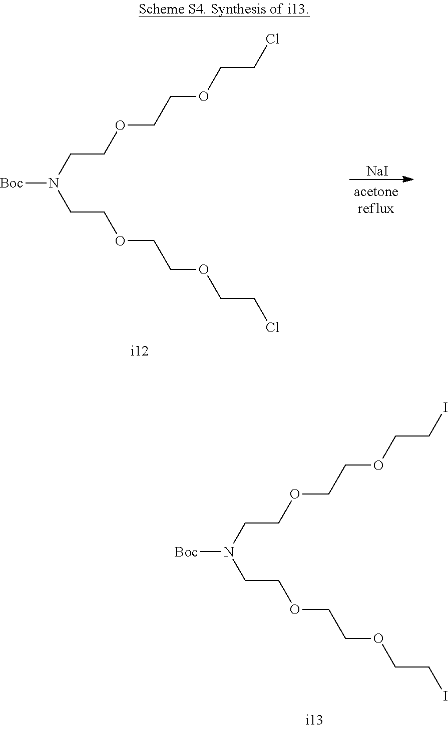 [SMILES: CC(C)(C)OC(=O)N(CCOCCOCCCl)CCOCCOCCCl ; "NaI" ; CC(C)(C)OC(=O)N(CCOCCOCCI)CCOCCOCCI]

### Compound i13

To a solution of 12 (19, 20) (837 mg, 2 mmol) in acetone (30 mL) was added NaI (6 g, 40 mmol). The reaction mixture was refluxed overnight. After evaporation of the solvent, water was added and the product was extracted with EtOAc. The combined organic extracts were washed with brine, dried over Na₂SO₄, filtered and the solvent was removed under reduced pressure to give compound i13 as a yellow oil (1.03 g, 1.7 mmol, 86%). The product was used without further purification.

¹H NMR (600 MHz, CDCl₃): δ=3.75 (t, J=6.9, 4H), 3.66-3.55 (12H), 3.50-3.41 (4H), 3.26 (t, J=6.9, 4H), 1.45 (s, 9H); ¹³C NMR (150 MHz, CDCl₃): δ=155.4, 79.6, 72.0, 70.4, 70.2, 69.9, 69.7, 47.8, 47.6, 28.5, 2.9; HR-MS (MALDI): Calcd. for C₁₇H₃₃NnaO₆⁺ [M+Na]⁺: 624.0289. found: 624.0295.

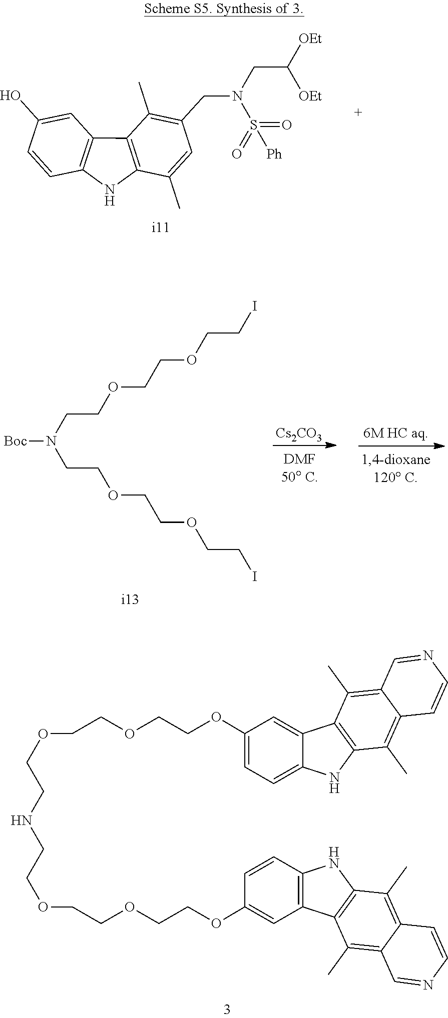 [SMILES: CCOC(CN(Cc1cc(C)c2[nH]c3ccc(O)cc3c2c1C)S(=O)(=O)c1ccccc1)OCC ; CC(C)(C)OC(=O)N(CCOCCOCCI)CCOCCOCCI ; "Cs2CO3" ; Cc1c2ccncc2c(C)c2c1[nH]c1ccc(OCCOCCOCCNCCOCCOCCOc3ccc4[nH]c5c(C)c6ccncc6c(C)c5c4c3)cc12]

### Compound 3

To a solution of i11 (496 mg, 1 mmol) and i13 (262 mg, 400 μmol) in DMF (4 mL) was added Cs₂CO₃ (522 mg, 1.6 mmol). The mixture was stirred at 50° C. overnight. The solvent was evaporated, and the product was extracted with EtOAc. The combined organic extracts were washed with sat. NaHCO₃, brine, and dried over Na₂SO₄. After solvent removal under reduced pressure, the crude product was obtained. To a solution of the crude product in 1,4-dioxane (10 mL) was added 6 M HCl aq. (2 mL), and the solution was heated at 120° C. for 1 h under microwave irradiation. The solution was concentrated, and the crude product was purified by HPLC (30-55% MeOH/H₂O, 0.1% TFA) to give 3 (TFA salt) as a red solid (84.3 mg, 75.8 μmol, 19%).

¹H NMR (400 MHz, DMSO-d₆): δ=11.80 (s, 2H), 9.77 (s, 2H), 8.68 (brs, 2H), 8.32 (d, J=7.0, 2H), 8.20 (d, J=7.0, 2H), 7.60 (d, J=2.3, 2H), 7.46 (d, J=8.7, 2H), 7.19 (dd, J=8.8, 2.4, 2H), 4.22-4.13 (4H), 3.89-3.80 (4H), 3.80-3.62 (12H), 3.29-3.18 (4H), 3.04 (s, 6H), 2.64 (s, 6H); ¹³C NMR (100 MHz, DMSO-d₆): δ=153.1, 144.4, 143.6, 137.0, 133.3, 133.2, 127.4, 125.4, 122.4, 119.1, 119.1, 117.0, 111.9, 109.7, 108.3, 69.8, 69.8, 69.2, 67.8, 65.7, 46.2, 14.6, 11.7; HR-MS (MALDI): Calcd. for C₄₆H₅₂N₅O₆⁺ [M+H]⁺: 770.3912. found: 770.3888.

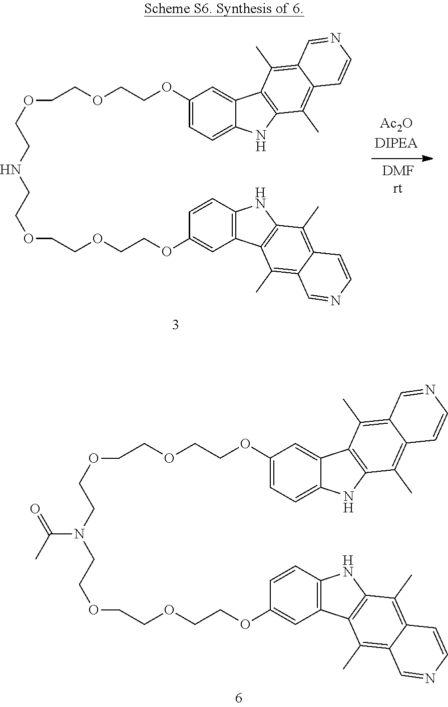 [SMILES: Cc1c2ccncc2c(C)c2c1[nH]c1ccc(OCCOCCOCCNCCOCCOCCOc3ccc4[nH]c5c(C)c6ccncc6c(C)c5c4c3)cc12 ; CC(=O)N(CCOCCOCCOc1ccc2[nH]c3c(C)c4ccncc4c(C)c3c2c1)CCOCCOCCOc1ccc2[nH]c3c(C)c4ccncc4c(C)c3c2c1]

### Compound 6

To a solution of the TFA salt of dimer 3 (5.6 mg, 5 μmol) and DIPEA (8.7 μL, 50 μmol) in DMF (300 μL) was added Ac₂O (1.1 μL, 15 μmol) in DMF (100 μL). The mixture was stirred at room temperature for 1 h. The solvent was evaporated, and the product was purified by HPLC (40-70% MeOH/H₂O, 0.1% TFA) to give 6 (TFA salt) as a yellow oil (2.7 mg, 2.6 μmol, 52%).

¹H NMR (600 MHz, CD₃OD): δ=9.37-9.29 (2H), 8.16-8.02 (4H), 7.37-7.26 (2H), 7.16-7.09 (2H), 7.05-6.95 (2H), 4.15-4.06 (4H), 3.98 (s, 2H), 3.95-3.86 (4H), 3.84-3.65 (16H), 2.82-2.72 (6H), 2.55-2.45 (6H), 2.20 (s, 3H); ¹³C NMR (150 MHz, CD₃OD): δ=174.1, 155.0, 155.0, 146.0, 146.0, 143.6, 138.2, 134.9, 134.9, 134.3, 127.8, 127.7, 127.1, 127.1, 123.7, 120.7, 120.7, 120.3, 120.3, 118.3, 118.2, 112.6, 112.6, 110.8, 110.8, 109.9, 109.8, 72.0, 72.0, 71.9, 71.7, 71.2, 70.5, 70.1, 69.6, 69.6, 55.1, 51.1, 47.7, 21.9, 15.0, 15.0, 11.9, 11.9; HR-MS (MALDI): Calcd. for C₄₈H₅₄N₅O₇+[M+H]⁺: 812.4018. found: 812.4016.

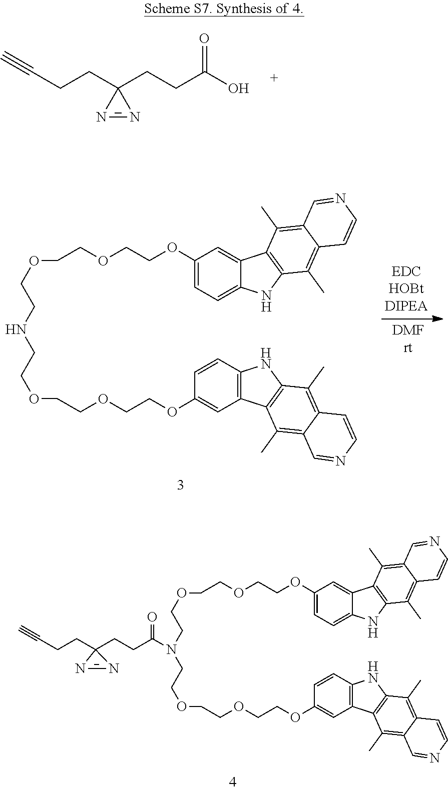 [SMILES: C#CCCC1(CCC(=O)O)N=N1 ; Cc1c2ccncc2c(C)c2c1[nH]c1ccc(OCCOCCOCCNCCOCCOCCOc3ccc4[nH]c5c(C)c6ccncc6c(C)c5c4c3)cc12 ; C#CCCC1(CCC(=O)N(CCOCCOCCOc2ccc3[nH]c4c(C)c5ccncc5c(C)c4c3c2)CCOCCOCCOc2ccc3[nH]c4c(C)c5ccncc5c(C)c4c3c2)N=N1]

### Compound 4

To a solution of the TFA salt of dimer 3 (5.6 mg, 5 μmol) and DIPEA (8.7 μL, 50 μmol) in DMF (200 μL) was added a solution of i4 (2.5 mg, 15 μmol), EDC (2.9 mg, 15 μmol), and HOBt (2.3 mg, 15 μmol) in DMF (200 μL). The mixture was stirred at room temperature overnight. The solvent was evaporated, and the product was purified by HPLC (40-70% MeOH/H₂O, 0.1% TFA) to give 4 (TFA salt) as a red solid (3.4 mg, 3.0 μmol, 59%).

¹H NMR (600 MHz, DMSO-d₆): δ=11.75 (s, 2H), 9.75 (s, 2H), 8.37-8.27 (m, 2H), 8.27-8.17 (m, 2H), 7.65 (d, J=1.8 Hz, 2H), 7.44 (d, J=8.7 Hz, 2H), 7.20 (dd, J=8.7, 2.1 Hz, 2H), 4.18 (m, 4H), 3.82 (m, 4H), 3.69-3.63 (4H), 3.63-3.56 (6H), 3.55-3.44 (6H), 3.06 (s, 6H), 2.79 (m, 1H), 2.65 (s, 6H), 2.19 (t, J=7.5 Hz, 2H), 1.93 (td, J=7.4, 2.6 Hz, 2H), 1.60 (t, J=7.5 Hz, 2H), 1.54 (t, J=7.4 Hz, 2H; ¹³C NMR (150 MHz, DMSO-d₆): δ=170.9, 153.2, 144.4, 143.6, 136.9, 133.3, 133.2, 127.4, 125.4, 122.5, 119.2, 119.1, 117.0, 111.9, 109.6, 108.5, 108.5, 83.2, 71.7, 70.0, 70.0, 69.8, 69.2, 69.2, 69.0, 68.4, 67.9, 48.0, 45.8, 31.5, 28.3, 27.8, 26.3, 14.7, 12.6, 11.8; HR-MS (MALDI): Calcd. for C₅₄H₆₀N₇O₇⁺[M+H]⁺: 918.4549; found: 918.4526.

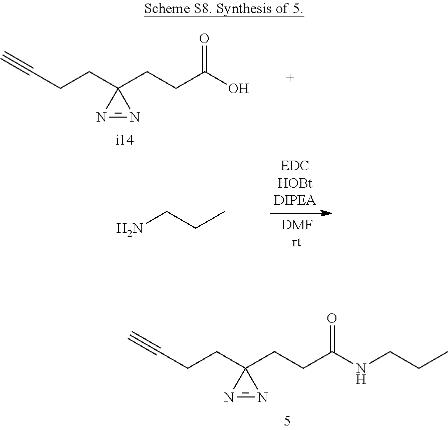

### Compound 5

The synthetic procedure for compound 5 is the same as described for the synthesis of compound 4.

¹H NMR (400 MHz, CDCl₃): δ=5.43 (br, 1H), 3.21 (m, 2H), 2.99 (td, J=7.3, 2.5 Hz, 2H), 1.95-1.89 (m, 2H), 1.87-1.82 (m, 2H), 1.65 (t, J=7.4 Hz, 2H), 1.52 (m, 2H), 0.92 (t, J=7.4 Hz, 3H); ¹³C NMR (100 MHz, CDCl₃): δ=171.1, 82.7, 69.2, 41.4, 32.4, 30.4, 29.7, 28.4, 22.8, 13.3, 11.3; HR-MS (ESI): Calcd. for C₁₂H₁₈N₃O₃⁻ [M+HCOO]⁻: 252.1354. found: 252.1347.

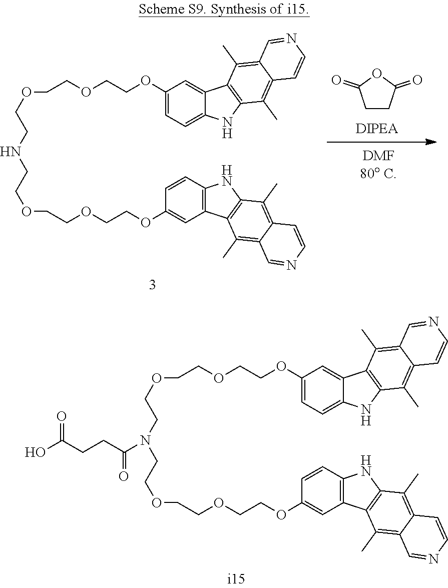 [SMILES: Cc1c2ccncc2c(C)c2c1[nH]c1ccc(OCCOCCOCCNCCOCCOCCOc3ccc4[nH]c5c(C)c6ccncc6c(C)c5c4c3)cc12 ; O=C1CCC(=O)O1 ; Cc1c2ccncc2c(C)c2c1[nH]c1ccc(OCCOCCOCCN(CCOCCOCCOc3ccc4[nH]c5c(C)c6ccncc6c(C)c5c4c3)C(=O)CCC(=O)O)cc12]

### Compound i15

To a solution of compound 3 (29 mg, 23.9 μmol as 3×TFA salt) and DIPEA (20.8 μL, 120 μmol) in anhydrous DMF (1 mL) was added succinic anhydride (2.9 mg, 28.7 μmol). After stirring at 80° C. for 1 h the solvent was evaporated and the crude product was purified by HPLC (15-40% acetonitrile/H₂O, 0.1% TFA) to give i15 (TFA salt) as a red solid (24 mg, 21.9 μmol, 91%).

¹H NMR (600 MHz, DMSO-d₆): δ=15.20 (br, 1H), 11.68 (s, 2H), 9.68 (s, 2H), 8.28 (d, J=6.9 Hz, 2H), 8.15 (d, J=6.9 Hz, 2H), 7.55 (m, 2H), 7.39 (dd, J=8.7, 1.1 Hz, 2H), 7.16 (dd, J=8.7, 2.3 Hz, 2H), 4.18-4.11 (4H), 3.86-3.78 (4H), 3.70-3.55 (12H), 3.55-3.48 (4H), 3.01-2.94 (6H), 2.63-2.55 (8H), 2.41 (m, 2H); ¹³C NMR (150 MHz, DMSO-d₆): δ=174.0, 171.3, 158.3 (q, J=34.6 Hz), 153.1, 144.3, 143.4, 136.9, 133.2, 133.1, 127.3, 125.3, 122.3, 119.0, 119.0, 117.0, 116.2 (q, J=293.9 Hz), 111.8, 109.5, 108.3, 70.1, 70.0, 70.0, 69.8, 69.3, 69.2, 69.0, 68.5, 67.8, 47.9, 45.9, 29.1, 27.5, 14.6, 11.7; HRMS (MALDI) calculated for C₅₀H₅₆N₅O₉⁺[M+H]⁺: 870.4073. found: 870.4059.

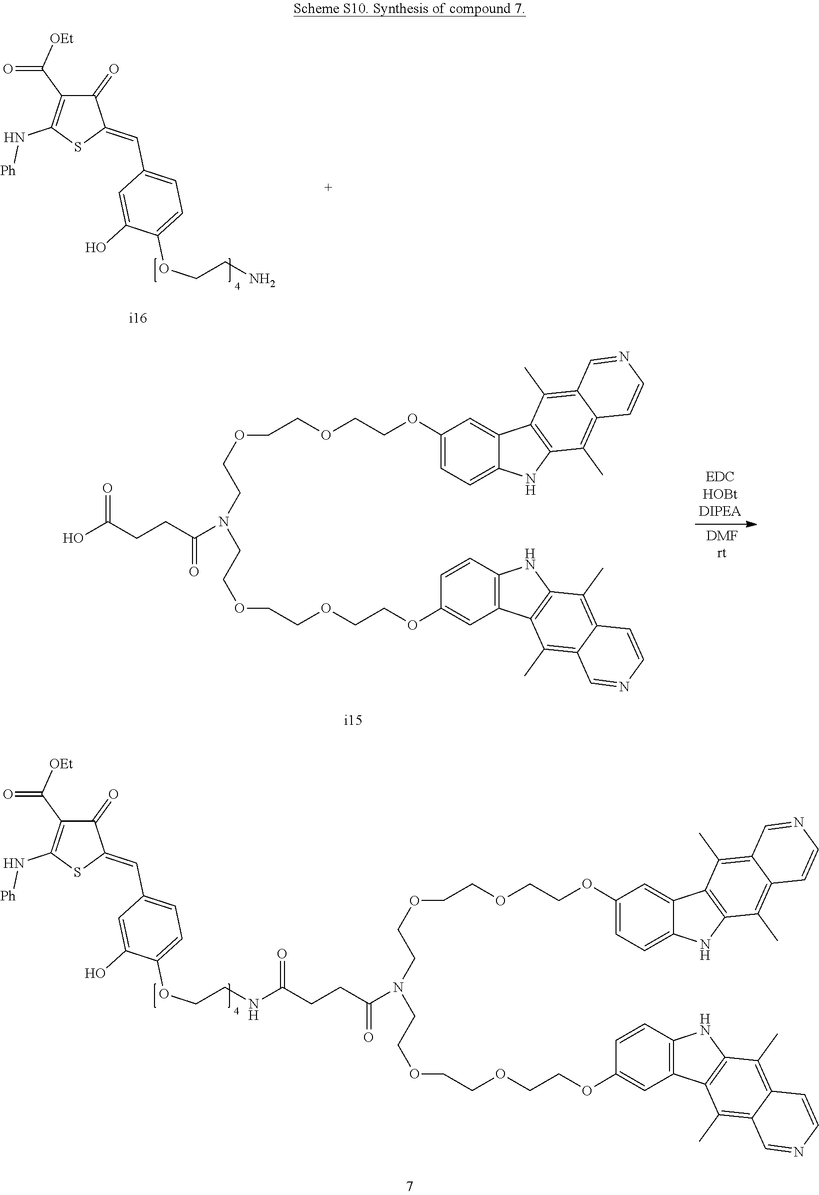

### Compound 7

To a mixture of acid i15 (10 mg, 9.1 μmol as 2TFA salt), EDC (5.2 mg, 27.3 μmol), HOBt (4.2 mg, 27.3 μmol) and DIPEA (7.9 μL, 46 μmol) in DMF (300 μL) was added a solution of 16 (10.8 mg, 27.3 μmol as a HCl salt) and DIPEA (9.6 μL, 54 μmol) in DMF (200 μL). The mixture was stirred at room temperature overnight. The solvent was evaporated, and the product was purified by HPLC (55-80% MeOH/H₂O, 0.10% TFA) to give 7 (TFA salt) as a red solid (7.2 mg, 4.4 μmol, 48%).

¹H NMR (600 MHz, DMSO-d₆): δ=11.72 (m, 2H), 11.20 (s, 1H), 9.72 (m, 2H), 8.30 (d, J=6.8 Hz, 2H), 8.20 (m, 2H), 7.84 (t, J=5.5 Hz, 1H), 7.62 (m, 2H), 7.52 (m, 2H), 7.47 (m, 2H), 7.45-7.39 (4H), 7.18 (m, 2H), 6.95 (d, J=8.4 Hz, 1H), 6.92-6.86 (2H), 4.26 (q, J=7.1 Hz, 2H), 4.16 (m, 4H), 4.06 (m, 2H), 3.97 (s, 1H), 3.81 (m, 4H), 3.75-3.41 (27H), 3.35 (t, J=5.9 Hz, 2H), 3.14 (q, J=5.8 Hz, 2H), 3.03 (s, 6H), 2.63 (s, 6H), 2.56 (t, J=7.0 Hz, 2H), 2.31 (t, J=7.0 Hz, 2H), 1.29 (t, J=7.1 Hz, 3H); ¹³C NMR (150 MHz, DMSO-d₆): δ=180.8, 175.2, 171.6, 164.9, 158.2 (q, J=36.0 Hz), 153.2, 148.7, 147.0, 144.4, 143.5, 137.5, 136.9, 133.3, 133.2, 129.9, 129.6, 128.1, 127.3, 126.2, 125.5, 125.4, 124.5, 123.0, 122.4, 119.1, 119.1, 117.0, 115.8, 113.6, 111.9, 109.6, 108.5, 108.4, 96.8, 70.1, 70.0, 70.0, 69.9, 69.8, 69.8, 69.7, 69.6, 69.3, 69.2, 69.1, 69.0, 68.8, 68.5, 67.9, 67.8, 67.8, 59.5, 55.1, 47.9, 45.9, 38.6, 30.5, 27.9, 14.6, 14.4, 11.7; HRMS (ESI) calculated for C₇₈H₈₈N₇O₁₆S⁺[M+H]⁺: 1410.6002; found: 1410.6001.

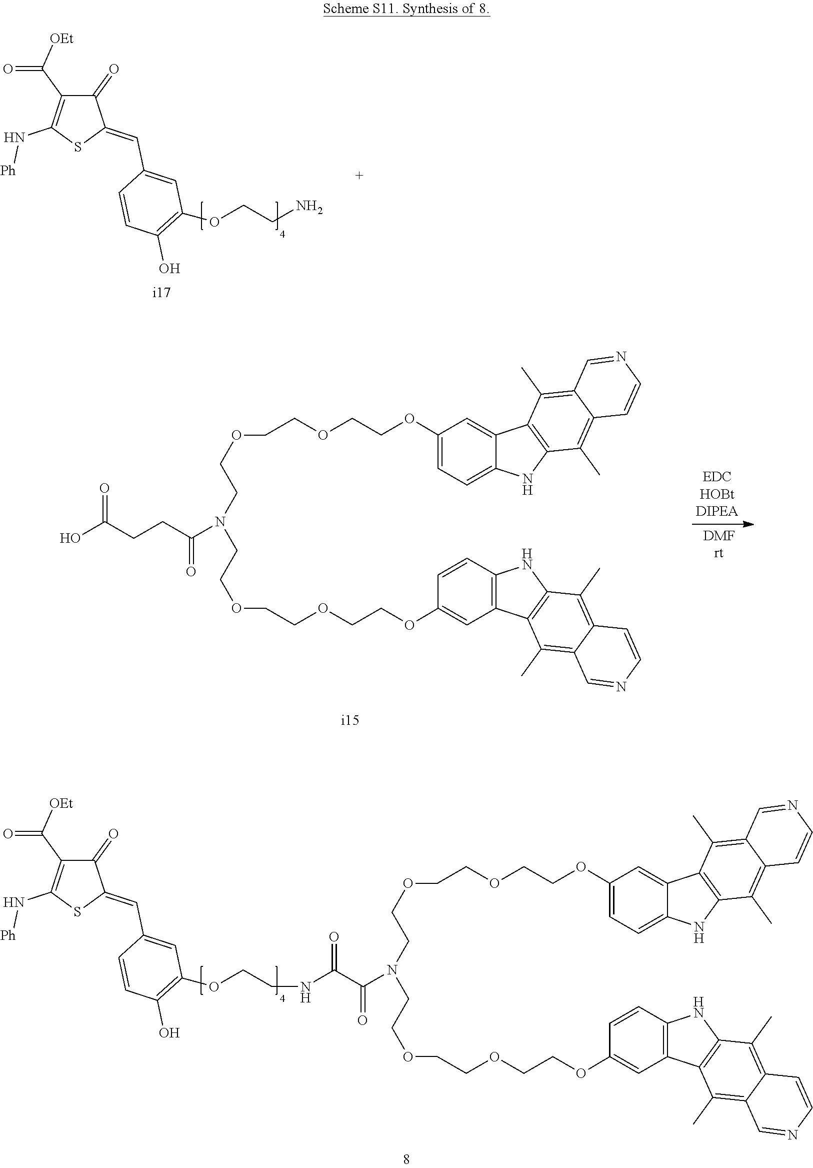

### Compound 8

To a mixture of acid i15 (12 mg, 11 μmol as 2 TFA salt), EDC (6.3 mg, 33 μmol), HOBt (5 mg, 33 μmol) and DIPEA (9.6 μL, 55 μmol) in DMF (300 μL) was added a solution of 17 (19.6 mg, 33 μmol as a HCl salt) and DIPEA (9.6 μL, 55 μmol) in DMF (200 μL). The mixture was stirred at room temperature overnight. The solvent was evaporated, and the product was purified by HPLC (55-80% MeOH/H₂O, 0.1% TFA) to give 8 (TFA salt) as a red solid (11.4 mg, 7.0 μmol, 63%).

¹H NMR (600 MHz, CD₃OD): δ=9.27 (s, 1H), 9.24 (s, 1H), 8.09-8.02 (2H), 8.00-7.93 (2H), 7.43 (m, 2H), 7.36 (m, 1H), 7.20-7.15 (4H), 7.08-7.01 (2H), 6.93-6.87 (3H), 6.53 (d, J=8.1 Hz, 1H), 6.44 (dd, J=8.2, 1.9 Hz, 1H), 6.31 (d, J=1.7 Hz, 1H), 4.28 (q, J=7.1 Hz, 2H), 4.03-3.98 (4H), 3.86-3.81 (4H), 3.78-3.59 (29H), 3.52 (t, J=5.5 Hz, 2H), 3.32 (m, 2H), 2.77 (t, J=7.1 Hz, 2H), 2.70-2.64 (6H), 2.50 (t, J=7.1 Hz, 2H), 2.43 (s, 3H), 2.41 (s, 3H), 1.35 (t, J=7.1 Hz, 3H); ¹³C NMR (150 MHz, CD₃OD): δ=183.4, 176.1, 175.0, 174.8, 166.7, 154.9, 154.8, 150.4, 147.8, 146.0, 145.9, 143.5, 143.4, 138.5, 138.1, 138.1, 134.9, 134.8, 134.2, 134.2, 132.3, 130.8, 128.9, 127.7, 127.7, 127.1, 126.0, 125.7, 125.0, 124.1, 123.7, 123.7, 120.6, 120.6, 120.2, 120.2, 118.0, 117.9, 116.9, 116.0, 112.6, 110.9, 110.9, 109.8, 109.7, 98.4, 71.9, 71.9, 71.9, 71.7, 71.6, 71.6, 71.3, 71.1, 70.5, 70.3, 70.1, 69.5, 69.4, 69.2, 61.4, 50.0, 47.6, 40.5, 32.2, 29.8, 15.1, 14.9, 11.9; HRMS (ESI) calculated for C₇₈H₈₈N₇O₁₆S⁺[M+H]⁺: 1410.6002. found: 1410.6010.

TABLE 1
Binding parameters of compounds 1a, 1b, 2, 3, 6 and 7 to various nucleic
acids obtained via biolayer interferometry and in vitro IC₅₀s.

| | Nucleic | K_{d, 1} | k_{on, 1} | k_{off, 1} | K_{d, 2} | k_{on, 1} | k_{off, 1} |
|---|---|---|---|---|---|---|---|
| Cpd | acid | (M) | (1/M × s) | (1/s) | (M) | (1/M × s) | (1/s) |
| 1a | r(G₄C₂)₈ | 60(±3) × | 0.3(±0.39) × | 1.4(±0.2) × | 40(±2) × | 1.7(±0.2) × | 3.5(±0.3) × |
| | | 10⁻⁸ | 10⁵ | 10⁻³ | 10⁻⁷ | 10⁴ | 10⁻² |
| 1b | r(G₄C₂)₈ | 1100(±140) × | 0.08(±0.003) × | 20(±1) × | N/A | N/A | N/A |
| | | 10⁻⁸ | 10⁵ | 10⁻³ | | | |
| 2 | r(G₄C₂)₈ | 5(±0.8) × | 1.4(±0.4) × | 1.8(±0.1) × | 1.2(±0.2) × | 2.5(±0.5) × | 2.4(±0.1) × |
| | | 10⁻⁸ | 10⁵ | 10⁻³ | 10⁻⁷ | 10⁵ | 10⁻² |
| | d(G₄C₂)₈ | 130(±27) × | 0.1(±0.04) × | 17(±5) × | 380(±22) × | 0.06(±0.005) × | 90(±18) × |
| | | 10⁻⁸ | 10⁵ | 10⁻³ | 10⁻⁷ | 10⁵ | 10⁻³ |
| | r(G₂C₂)₁₀ | 600(±228) × | 0.15(±0.06) × | 14(±8) × | N/A | N/A | N/A |
| | | 10⁻⁸ | 10⁵ | 10⁻² | | | |
| | r(G₂C₄)₈ | 3200(±900) × | 0.2(±0.02) × | 200(±10) × | 1200(±300) × | 0.005(±0.0005) × | 7.0(±0.9) × |
| | | 10⁻⁸ | 10⁵ | 10⁻³ | 10⁻⁷ | 10⁵ | 10⁻² |
| 3 | r(G₄C₂)₈ | 0.4(±0.07) × | 0.2(±0.07) × | 0.08(±0.03) × | 9(±0.3) × | 0.03(±0.003) × | 1.9(±0.1) × |
| | | 10⁻⁸ | 10⁵ | 10⁻³ | 10⁻⁷ | 10⁵ | 10⁻³ |
| | d(G₄C₂)₈ | 90(±13) × | 0.09(±0.02) × | 50(±34) × | 36(±16) × | 0.09(±0.04) × | 18(±7) × |
| | | 10⁻⁸ | 10⁵ | 10⁻³ | 10⁻⁷ | 10⁵ | 10⁻³ |
| | r(G₂C₂)₁₀ | 80(±33) × | 0.06(±0.002) × | 4(±1.5) × | N/A | N/A | N/A |
| | | 10⁻⁸ | 10⁵ | 10⁻³ | | | |
| | r(G₂C₄)₈ | 40(±14) × | 0.1(±0.03) × | 3(±0.3) × | N/A | N/A | N/A |
| | | 10⁻⁸ | 10⁵ | 10⁻³ | | | |
| 6 | r(G₄C₂)₈ | 20(±0.4) × | 0.01(±0.0002) × | 0.2(±0.002) × | 106(±8) × | 0.01(±0.001) × | 1.2(±0.003) × |
| | | 10⁻⁸ | 10⁵ | 10⁻³ | 10⁻⁷ | 10⁵ | 10⁻² |

In vitro IC₅₀ by TR-FRET

| Cmpd | IC₅₀ (mM) |
|---|---|
| 2 | 1.8 ± 0.09 |
| 3 | 0.9 ± 0.06 |
| 4 | 2.7 ± 0.03 |
| 7 | 3.4 ± 0.13 |

TABLE 2
Sequences of primers used in this study.

| Primer Name | Sequence (5′ to 3′) (SEQ ID NO:) |
|---|---|
| GFP (fwd) | GCACGACTTCTTCAAGTCCGCCATGCC (1) |
| GFP (rev) | GCGGATCTTGAAGTTCACCTTGATGCC (2) |
| GAPDH (fwd) | GAAGGTGAAGGTCGGAGTC (3) |
| GAPDH (rev) | GAAGATGGTGATGGGATTTC (4) |
| β-actin (fwd) | GATTACTGCTCTGGCTCCTAGCA (5) |
| β-actin (rev) | GCTCAGGAGGAGCAATGATCTT (6) |
| C9orf72 exon 2-3 (fwd) | ACTGGAATGGGGATCGCAGCA (7) |
| C9orf72 exon 2-3 (rev) | ACCCTGATCTTCCATTCTCTCTGTGCC (8) |
| C9orf72 intron 1 (fwd) | ACGCCTGCACAATTTCAGCCCAA (9) |
| C9orf72 intron 1 (rev) | CAAGTCTGTGTCATCTCGGAGCTG (10) |
| C9orf72 exon 1b (fwd) | CTGCGGTTGCGGTGCCTGC (11) |
| C9orf72 exon 1b (rev) | AGCTGGAGATGGCGGTGGGC (12) |
| UNKL isoform 1 (fwd) | CTGCTCCAAGTACAACGAAGCC (13) |
| UNKL isoform 1 (rev) | TCTGTCTCGTGGATGCAGGTTC (14) |
| Enoyl-CoA (fwd) | GCTGCCAGCAAGGATGACTCAA (15) |
| Enoyl-CoA (rev) | GCTTTCTCCTCTACTCCACCAG (16) |
| CAMP (fwd) | GACACAGCAGTCACCAGAGGAT (17) |
| CAMP (rev) | TCACAACTGATGTCAAAGGAGCC (18) |
| XYLT1 (fwd) | TGATGCCTGAGAAGGTGACTCG (19) |
| XYLT1 (rev) | CACCAGGACAAAGGCGATTCTG (20) |
| RNA BP10 (fwd) | GGCATCTACCAACAATCAGCCG (21) |
| RNA BP10 (rev) | GGAGAGCAGAACTAGGATGGGT (22) |
| Rab-40C (fwd) | GTACGCCTACAGTAACGGGATC (23) |
| Rab-40C (rev) | CTGGAGTAGGACCTGAAGATGG (24) |
| SOCS1 (fwd) | TTCGCCCTTAGCGTGAAGATGG (25) |
| SOCS1 (rev) | TAGTGCTCCAGCAGCTCGAAGA (26) |
| USP7 (fwd) | GTCACGATGACGACCTGTCTGT (27) |
| USP7 (rev) | GTAATCGCTCCACCAACTGCTG (28) |

TABLE 3
Sequences of ASOs and LNA oligonucleotides used in this study.

| Name | Sequence (5′ to 3′)^{a} |
|---|---|
| G₄C₂-ASO | mG*mG*mC*C*C*C*G*G*C*C*C*C*G*G*C*C*C*mC*mG*mG |
| C9orf72-ASO | mU*mA*mC*A*G*G*C*T*G*C*G*G*T*T*G*T*T*mU*mC*m |
| Control-ASO | mC*mC*mU*T*C*C*C*T*G*A*A*G*G*T*T*C*C*mU* |
| | mC*mC |
| Cy5-G₄C₂-Cy3 | Cy5-CCGGGGCCGGGGCCGGGGCCGGGG-Cy3 (SEQ ID NO: 29) |
| G₄C₂-LNA^{b} | G*G*C*C*C*C*G*G*C*C*C*C*G*G |
| Control-LNA^{b} | T*A*A*C*A*C*G*T*C*T*A*T*A*C |
| Sense FISH | TYE563-CCCCGGCCCCGGCCCC-TYE563 (SEQ ID NO: 30) |
| Probe | |

^{a}m indicates 2′-O-methyl residue; * indicates LNA residue
^{b}miRCURY LNA (Qiagen)

TABLE 4
Atom names, types, and charges used to define 1
RNA-binding modules and the PEG linker in NMR spectroscopic and
computational modeling studies (see FIG. 7-B & D, and FIG. 2-A).
MM-PBSA results for all clusters of 3 bound to r(G₄C₂).

| | | | | | |
|---|---|---|---|---|---|
| Atom | Atom | | Atom | Atom | |
| Name | Type | Charge | Name | Type | Charge |

1

| | | | | | |
|---|---|---|---|---|---|
| N1 | nb | −0.694785 | H7 | hc | 0.115552 |
| C1 | ca | 0.370507 | H8 | hc | 0.115552 |
| H1 | h4 | 0.060190 | H9 | hc | 0.115552 |
| C2 | ca | −0.468835 | N2 | na | −0.462081 |
| H2 | ha | 0.160170 | H10 | hn | 0.382727 |
| C3 | ca | 0.274608 | C12 | ca | 0.118935 |
| C4 | ca | −0.328629 | C13 | cp | 0.194840 |
| C5 | ca | 0.494033 | C14 | ca | −0.286734 |
| H3 | h4 | 0.028587 | H11 | ha | 0.188878 |
| C6 | ca | −0.080071 | C15 | ca | −0.252541 |
| C7 | c3 | −0.165494 | H12 | ha | 0.151765 |
| H4 | hc | 0.063582 | C16 | ca | 0.359798 |
| H5 | hc | 0.063582 | O1 | os | −0.388618 |
| H6 | hc | 0.063582 | C17 | c3 | 0.240640 |
| C8 | ca | 0.102523 | H14 | h1 | 0.003191 |
| C9 | cp | −0.119054 | H15 | h1 | 0.003191 |
| C10 | ca | 0.132438 | C18 | ca | −0.393231 |
| C11 | c3 | −0.343684 | H16 | ha | 0.179334 |

NH-modified PEG Linker

| | | | | | |
|---|---|---|---|---|---|
| C1 | c3 | −0.136726 | H24 | hn | 0.278145 |
| H2 | hc | 0.060502 | C25 | c3 | −0.074542 |
| H3 | hc | 0.040100 | H26 | hx | 0.125404 |
| H4 | hc | 0.046696 | H27 | hx | 0.125404 |
| C5 | c3 | 0.159266 | C28 | c3 | 0.075797 |
| H6 | h1 | 0.039150 | H29 | h1 | 0.080461 |
| H7 | h1 | 0.039150 | H30 | h1 | 0.080461 |
| O8 | os | −0.400564 | O31 | os | −0.383822 |
| C9 | c3 | 0.179397 | C32 | c3 | 0.043488 |
| H10 | h1 | 0.030255 | H33 | h1 | 0.078204 |
| H11 | h1 | 0.030255 | H34 | h1 | 0.078204 |
| C12 | c3 | 0.043488 | C35 | c3 | 0.179397 |
| H13 | h1 | 0.078204 | H36 | h1 | 0.030255 |
| H14 | h1 | 0.078204 | H37 | h1 | 0.030255 |
| O15 | os | −0.383822 | O38 | os | −0.400564 |
| C16 | c3 | 0.051987 | C39 | c3 | 0.159266 |
| H17 | h1 | 0.080461 | H40 | h1 | 0.039150 |
| H18 | h1 | 0.080461 | H41 | h1 | 0.039150 |
| C19 | c3 | −0.062791 | C42 | c3 | −0.179824 |
| H20 | hx | 0.125404 | H43 | hc | 0.057284 |
| H21 | hx | 0.125404 | H44 | hc | 0.035351 |
| N22 | n4 | −0.156167 | H45 | hc | 0.076618 |
| H23 | hn | 0.278145- | | | |

| Cluster | Size of cluster as a fraction | Δ_{GMM-PBSA} |
|---|---|---|
| number | of the total trajectory | (kcal/mol) |
| 1 | 0.364 | −68.23 |
| 2 | 0.316 | −63.60 |
| 3 | 0.136 | −61.07 |

TABLE 5
Summary of cell line demographic information and the
corresponding figures in which they were used.

| Identifier | Cell Type | Diagnosis | Sex | Age | Source | Experiments |
|---|---|---|---|---|---|---|
| CRL-3216 | HEK293T | Control | Female | Fetus | ATCC | 2E, 2H, 4A-4E, |
| | | | | | | 8A, 10, 12B |
| ND11583 | LCL | C9orf72 | Male | 59 | Coriell | 2D, 2F, 5A-B, |
| | | | | | | 5D-F, 7H, 8A, |
| | | | | | | 9, 12A-C |
| ND12438 | LCL | C9orf72 | Male | 65 | Coriell | 2F, 5A, 5D |
| ND09492 | LCL | C9orf72 | Male | 52 | Coriell | 2F, 5A, 5D |
| GM07491 | LCL | Healthy | Male | 17 | Coriell | 5C |
| CS0BUU | iPSC | C9orf72 | Female | 63 | Cedars Sinai | 2D, 2G, 5G-K, |
| | | | | | | 8A, 8C-D, 12A-B, |
| | | | | | | 12D, 12E, 14B-C, |
| CS0NKC | iPSC | C9orf72 | Female | 60 | Cedars Sinai | 2G, 5G, 5H, 17C |
| CS2YNL | iPSC | C9orf72 | Male | 60 | Cedars Sinai | 2G, 5G, 5H, 14C |
| CS7VCZ | iPSC | C9orf72 | Male | 64 | Cedars Sinai | 2G, 5G-J, 14C |
| CS8PAA | iPSC | Healthy | Female | 58 | Cedars Sinai | 14A, 14D |
| CS9XH7 | iPSC | Healthy | Male | 53 | Cedars Sinai | 14A |
| Edi044-A | iPSC | Healthy | Female | 80 | Cedars Sinai | 14A, 14D |
| CS1ATZ | iPSC | Healthy | Male | 60 | Cedars Sinai | 14A |

TABLE 6
Sex, genotype, and age of mice used in in vivo studies.

| Mouse | Sex | Genotype | Age | Treatment |
|---|---|---|---|---|
| 1 | M | (WT) | 22 | Compound |
| 2 | M | (WT) | 22 | Compound |
| 3 | F | (WT) | 22 | Compound |
| 4 | F | (WT) | 20 | Compound |
| 5 | M | (WT) | 20 | Compound |
| 6 | F | (WT) | 18 | Compound |
| 7 | M | (+/+) | 22 | Compound |
| 8 | M | (+/+) | 22 | Compound |
| 9 | M | (+/+) | 22 | Compound |
| 10 | F | (+/+) | 22 | Compound |
| 11 | F | (+/+) | 22 | Compound |
| 12 | F | (+/+) | 25 | Compound |
| 13 | F | (WT) | 22 | Vehicle |
| 14 | M | (WT) | 22 | Vehicle |
| 15 | M | (WT) | 22 | Vehicle |
| 16 | M | (WT) | 20 | Vehicle |
| 17 | F | (WT) | 20 | Vehicle |
| 18 | F | (WT) | 18 | Vehicle |
| 19 | M | (+/+) | 22 | Vehicle |
| 20 | M | (+/+) | 22 | Vehicle |
| 21 | M | (+/+) | 22 | Vehicle |
| 22 | M | (+/+) | 22 | Vehicle |
| 23 | F | (+/+) | 22 | Vehicle |
| 24 | F | (+/+) | 25 | Vehicle |

3 Week Treatment Period

| | | | | |
|---|---|---|---|---|
| 1 | M | (WT) | 22 | 33 nmol 7 |
| 2 | F | (+/+) | 25 | 33 nmol 7 |
| 3 | F | (+/+) | 25 | Vehicle |
| 4 | M | (WT) | 22 | 33 nmol 7 |
| 5 | F | (WT) | 22 | 33 nmol 7 |
| 6 | M | (+/+) | 22 | 33 nmol 7 |
| 7 | M | (+/+) | 22 | 33 nmol 7 |
| 8 | M | (+/+) | 22 | 33 nmol 7 |
| 9 | F | (+/+) | 22 | 33 nmol 7 |
| 10 | F | (+/+) | 22 | 33 nmol 7 |
| 11 | F | (WT) | 22 | Vehicle |
| 12 | M | (WT) | 22 | Vehicle |
| 13 | M | (WT) | 22 | Vehicle |
| 14 | M | (+/+) | 22 | Vehicle |
| 15 | M | (+/+) | 22 | Vehicle |
| 16 | M | (+/+) | 22 | Vehicle |
| 17 | M | (+/+) | 22 | Vehicle |
| 18 | F | (+/+) | 22 | Vehicle |
| 19 | F | (WT) | 20 | 33 nmol 7 |
| 20 | M | (WT) | 20 | 33 nmol 7 |
| 21 | M | (WT) | 20 | Vehicle |
| 22 | F | (WT) | 20 | Vehicle |
| 23 | F | (WT) | 18 | 33 nmol 7 |
| 24 | F | (WT) | 18 | Vehicle |

6 Week Treatment Period

| | | | | |
|---|---|---|---|---|
| 1 | M | (+) | 21 | 33 nmol 7 |
| 2 | M | (+) | 22 | 33 nmol 7 |
| 3 | M | (+) | 22 | 33 nmol 7 |
| 4 | M | (+) | 22 | Vehicle |
| 5 | M | (+) | 22 | Vehicle |
| 6 | M | (+) | 22 | Vehicle |
| 7 | M | (+) | 22 | Vehicle |
| 8 | M | (+) | 21 | 33 nmol 7 |
| 9 | F | (+) | 21 | Vehicle |
| 10 | F | (+) | 20 | 33 nmol 7 |
| 11 | F | (+) | 20 | 33 nmol 7 |
| 12 | F | (+) | 20 | Vehicle |
| 13 | M | (+) | 19 | 33 nmol 7 |
| 14 | F | (+) | 19 | 33 nmol 7 |
| 15 | F | (+) | 19 | 33 nmol 7 |
| 16 | M | (+) | 19 | 33 nmol 7 |
| 17 | M | (+) | 19 | Vehicle |
| 18 | M | (+) | 19 | Vehicle |
| 19 | F | (+) | 19 | Vehicle |
| 20 | F | (+) | 19 | Vehicle |
| 21 | F | (+) | 17 | 33 nmol 7 |
| 22 | F | (+) | 17 | Vehicle |
| 23 | M | (+) | 16 | 33 nmol 7 |
| 24 | M | (+) | 16 | Vehicle |

### REFERENCES

1. M. DeJesus-Hernandez, I. R. Mackenzie, B. F. Boeve, A. L. Boxer, M. Baker, N. J. Rutherford, A. M. Nicholson, N. A. Finch, H. Flynn, J. Adamson, N. Kouri, A. Wojtas, P. Sengdy, G. Y. Hsiung, A. Karydas, W. W. Seeley, K. A. Josephs, G. Coppola, D. H. Geschwind, Z. K. Wszolek, H. Feldman, D. S. Knopman, R. C. Petersen, B. L. Miller, D. W. Dickson, K. B. Boylan, N. R. Graff-Radford, R. Rademakers, Expanded GGGGCC hexanucleotide repeat in noncoding region of C9ORF72 causes chromosome 9p-linked FTD and ALS. Neuron 72, 245-256 (2011).
2. A. E. Renton, E. Majounie, A. Waite, J. Simon-Sanchez, S. Rollinson, J. R. Gibbs, J. C. Schymick, H. Laaksovirta, J. C. van Swieten, L. Myllykangas, H. Kalimo, A. Paetau, Y. Abramzon, A. M. Remes, A. Kaganovich, S. W. Scholz, J. Duckworth, J. Ding, D. W. Harmer, D. G. Hernandez, J. O. Johnson, K. Mok, M. Ryten, D. Trabzuni, R. J. Guerreiro, R. W. Orrell, J. Neal, A. Murray, J. Pearson, I. E. Jansen, D. Sondervan, H. Seelaar, D. Blake, K. Young, N. Halliwell, J. B. Callister, G. Toulson, A. Richardson, A. Gerhard, J. Snowden, D. Mann, D. Neary, M. A. Nalls, T. Peuralinna, L. Jansson, V. M. Isoviita, A. L. Kaivorinne, M. Holtta-Vuori, E. Ikonen, R. Sulkava, M. Benatar, J. Wuu, A. Chio, G. Restagno, G. Borghero, M. Sabatelli, D. Heckerman, E. Rogaeva, L. Zinman, J. D. Rothstein, M. Sendtner, C. Drepper, E. E. Eichler, C. Alkan, Z. Abdullaev, S. D. Pack, A. Dutra, E. Pak, J. Hardy, A. Singleton, N. M. Williams, P. Heutink, S. Pickering-Brown, H. R. Morris, P. J. Tienari, B. J. Traynor, A hexanucleotide repeat expansion in C9ORF72 is the cause of chromosome 9p21-linked ALS-FTD. Neuron 72, 257-268 (2011).
3. J. Beck, M. Poulter, D. Hensman, J. D. Rohrer, C. J. Mahoney, G. Adamson, T. Campbell, J. Uphill, A. Borg, P. Fratta, R. W. Orrell, A. Malaspina, J. Rowe, J. Brown, J. Hodges, K. Sidle, J. M. Polke, H. Houlden, J. M. Schott, N. C. Fox, M. N. Rossor, S. J. Tabrizi, A. M. Isaacs, J. Hardy, J. D. Warren, J. Collinge, S. Mead, Large C9orf72 hexanucleotide repeat expansions are seen in multiple neurodegenerative syndromes and are more frequent than expected in the UK population. Am. J. Hum. Genet. 92, 345-353 (2013).
4. M. van Blitterswijk, M. DeJesus-Hernandez, E. Niemantsverdriet, M. E. Murray, M. G. Heckman, N. N. Diehl, P. H. Brown, M. C. Baker, N. A. Finch, P. O. Bauer, G. Serrano, T. G. Beach, K. A. Josephs, D. S. Knopman, R. C. Petersen, B. F. Boeve, N. R. Graff-Radford, K. B. Boylan, L. Petrucelli, D. W. Dickson, R. Rademakers, Association between repeat sizes and clinical and pathological characteristics in carriers of C9ORF72 repeat expansions (Xpansize-72): a cross-sectional cohort study. Lancet Neurol. 12, 978-988 (2013).
5. P. E. Ash, K. F. Bieniek, T. F. Gendron, T. Caulfield, W. L. Lin, M. Dejesus-Hernandez, M. M. van Blitterswijk, K. Jansen-West, J. W. Paul, 3^{rd}, R. Rademakers, K. B. Boylan, D. W. Dickson, L. Petrucelli, Unconventional translation of C9ORF72 GGGGCC expansion generates insoluble polypeptides specific to c9FTD/ALS. Neuron 77, 639-646 (2013).
6. K. Mori, T. Arzberger, F. A. Grasser, I. Gijselinck, S. May, K. Rentzsch, S. M. Weng, M. H. Schludi, J. van der Zee, M. Cruts, C. Van Broeckhoven, E. Kremmer, H. A. Kretzschmar, C. Haass, D. Edbauer, Bidirectional transcripts of the expanded C9orf72 hexanucleotide repeat are translated into aggregating dipeptide repeat proteins. Acta Neuropathol. 126, 881-893 (2013).
7. K. Mori, S. M. Weng, T. Arzberger, S. May, K. Rentzsch, E. Kremmer, B. Schmid, H. A. Kretzschmar, M. Cruts, C. Van Broeckhoven, C. Haass, D. Edbauer, The C9orf72 GGGGCC repeat is translated into aggregating dipeptide-repeat proteins in FTLD/ALS. Science 339, 1335-1338 (2013).
8. T. Zu, Y. Liu, M. Banez-Coronel, T. Reid, O. Pletnikova, J. Lewis, T. M. Miller, M. B. Harms, A. E. Falchook, S. H. Subramony, L. W. Ostrow, J. D. Rothstein, J. C. Troncoso, L. P. Ranum, RAN proteins and RNA foci from antisense transcripts in C9ORF72 ALS and frontotemporal dementia. Proc. Natl. Acad. Sci. U.S.A 110, E4968-4977 (2013).
9. T. F. Gendron, K. F. Bieniek, Y. J. Zhang, K. Jansen-West, P. E. Ash, T. Caulfield, L. Daughrity, J. H. Dunmore, M. Castanedes-Casey, J. Chew, D. M. Cosio, M. van Blitterswijk, W. C. Lee, R. Rademakers, K. B. Boylan, D. W. Dickson, L. Petrucelli, Antisense transcripts of the expanded C9ORF72 hexanucleotide repeat form nuclear RNA foci and undergo repeat-associated non-ATG translation in c9FTD/ALS. Acta Neuropathol. 126, 829-844 (2013).
10. J. Chew, C. Cook, T. F. Gendron, K. Jansen-West, G. Del Rosso, L. M. Daughrity, M. Castanedes-Casey, A. Kurti, J. N. Stankowski, M. D. Disney, J. D. Rothstein, D. W. Dickson, J. D. Fryer, Y. J. Zhang, L. Petrucelli, Aberrant deposition of stress granule-resident proteins linked to C9orf72-associated TDP-43 proteinopathy. Mol. Neurodegener. 14, 9 (2019).
11. C. Lagier-Tourenne, M. Baughn, F. Rigo, S. Sun, P. Liu, H. R. Li, J. Jiang, A. T. Watt, S. Chun, M. Katz, J. Qiu, Y. Sun, S. C. Ling, Q. Zhu, M. Polymenidou, K. Drenner, J. W. Artates, M. McAlonis-Downes, S. Markmiller, K. R. Hutt, D. P. Pizzo, J. Cady, M. B. Harms, R. H. Baloh, S. R. Vandenberg, G. W. Yeo, X. D. Fu, C. F. Bennett, D. W. Cleveland, J. Ravits, Targeted degradation of sense and antisense C9orf72 RNA foci as therapy for ALS and frontotemporal degeneration. Proc. Natl. Acad. Sci. U.S.A 110, E4530-4539 (2013).
12. Z. F. Wang, A. Ursu, J. L. Childs-Disney, R. Guertler, W. Y. Yang, V. Bernat, S. G. Rzuczek, R. Fuerst, Y. J. Zhang, T. F. Gendron, I. Yildirim, B. G. Dwyer, J. E. Rice, L. Petrucelli, M. D. Disney, The hairpin form of r(G4C2)exp in c9ALS/FTD is repeat-associated non-ATG translated and a target for bioactive small molecules. Cell Chem. Biol. 26, 179-190.e112 (2019).
13. Z. Su, Y. Zhang, T. F. Gendron, P. O. Bauer, J. Chew, W. Y. Yang, E. Fostvedt, K. Jansen-West, V. V. Belzil, P. Desaro, A. Johnston, K. Overstreet, S. Y. Oh, P. K. Todd, J. D. Berry, M. E. Cudkowicz, B. F. Boeve, D. Dickson, M. K. Floeter, B. J. Traynor, C. Morelli, A. Ratti, V. Silani, R. Rademakers, R. H. Brown, J. D. Rothstein, K. B. Boylan, L. Petrucelli, M. D. Disney, Discovery of a biomarker and lead small molecules to target r(GGGGCC)-associated defects in c9FTD/ALS. Neuron 84, 239 (2014).
14. M. G. Costales, H. Aikawa, Y. Li, J. L. Childs-Disney, D. Abegg, D. G. Hoch, S. P. Velagapudi, Y. Nakai, T. Khan, K. W. Wang, I. Yildirim, A. Adibekian, E. T. Wang, M. D. Disney, Small-molecule targeted recruitment of a nuclease to cleave an oncogenic RNA in a mouse model of metastatic cancer. Proc. Natl. Acad. Sci. U.S.A 117, 2406-2411 (2020).
15. K. Reddy, B. Zamiri, S. Y. Stanley, R. B. Macgregor, Jr., C. E. Pearson, The disease-associated r(GGGGCC)n repeat from the C9orf72 gene forms tract length-dependent uni- and multimolecular RNA G-quadruplex structures. J. Biol. Chem. 288, 9860-9866 (2013).
16. M. D. Disney, A. M. Winkelsas, S. P. Velagapudi, M. Southern, M. Fallahi, J. L. Childs-Disney, Inforna 2.0: a platform for the sequence-based design of small molecules targeting structured RNAs. ACS Chem. Biol. 11, 1720-1728 (2016).
17. S. P. Velagapudi, S. M. Gallo, M. D. Disney, Sequence-based design of bioactive small molecules that target precursor microRNAs. Nat. Chem. Biol. 10, 291-297 (2014).
18. M. D. Disney, Targeting RNA with small molecules to capture opportunities at the Intersection of chemistry, biology, and medicine. J Am. Chem. Soc. 141, 6776-6790 (2019).
19. L. Guan, M. D. Disney, Covalent small-molecule-RNA complex formation enables cellular profiling of small-molecule-RNA interactions. Angew. Chem. Int. Ed. Engl. 52, 10010-10013 (2013).
20. S. G. Rzuczek, L. A. Colgan, Y. Nakai, M. D. Cameron, D. Furling, R. Yasuda, M. D. Disney, Precise small-molecule recognition of a toxic CUG RNA repeat expansion. Nat. Chem. Biol. 13, 188-193 (2017).
21. J. L. Childs-Disney, T. Tran, B. R. Vummidi, S. P. Velagapudi, H. S. Haniff, Y. Matsumoto, G. Crynen, M. R. Southern, A. Biswas, Z.-F. Wang, T. L. Tellinghuisen, M. D. Disney, A massively parallel selection of small molecule-RNA motif binding partners informs design of an antiviral from sequence. Chem 4, 2384-2404 (2018).
22. P. Zhang, H.-J. Park, J. Zhang, E. Junnb, R. J. Andrews, S. P. Velagapudi, D. Abegg, K. Vishnu, M. G. Costales, J. L. Childs-Disney, A. Adibekian, W. N. Moss, M. M. Mouradian, M. D. Disney, Translation of intrinsically disordered protein α-synuclein is inhibited by a small molecule targeting a structure in its mRNA. Proc. Natl. Acad. Sci. U.S.A. 117, 1457-1467 (2020).
23. J. Jiang, Q. Zhu, T. F. Gendron, S. Saberi, M. McAlonis-Downes, A. Seelman, J. E. Stauffer, P. Jafar-Nejad, K. Drenner, D. Schulte, S. Chun, S. Sun, S. C. Ling, B. Myers, J. Engelhardt, M. Katz, M. Baughn, O. Platoshyn, M. Marsala, A. Watt, C. J. Heyser, M. C. Ard, L. De Muynck, L. M. Daughrity, D. A. Swing, L. Tessarollo, C. J. Jung, A. Delpoux, D. T. Utzschneider, S. M. Hedrick, P. J. de Jong, D. Edbauer, P. Van Damme, L. Petrucelli, C. E. Shaw, C. F. Bennett, S. Da Cruz, J. Ravits, F. Rigo, D. W. Cleveland, C. Lagier-Tourenne, Gain of toxicity from ALS/FTD-linked repeat expansions in C9ORF72 Is alleviated by antisense oligonucleotides targeting GGGGCC-containing RNAs. Neuron 90, 535-550 (2016).
24. C. J. Donnelly, P. W. Zhang, J. T. Pham, A. R. Haeusler, N. A. Mistry, S. Vidensky, E. L. Daley, E. M. Poth, B. Hoover, D. M. Fines, N. Maragakis, P. J. Tienari, L. Petrucelli, B. J. Traynor, J. Wang, F. Rigo, C. F. Bennett, S. Blackshaw, R. Sattler, J. D. Rothstein, RNA toxicity from the ALS/FTD C9ORF72 expansion is mitigated by antisense intervention. Neuron 80, 415-428 (2013).
25. T. F. Gendron, J. Chew, J. N. Stankowski, L. R. Hayes, Y. J. Zhang, M. Prudencio, Y. Carlomagno, L. M. Daughrity, K. Jansen-West, E. A. Perkerson, A. O'Raw, C. Cook, L. Pregent, V. Belzil, M. van Blitterswijk, L. J. Tabassian, C. W. Lee, M. Yue, J. Tong, Y. Song, M. Castanedes-Casey, L. Rousseau, V. Phillips, D. W. Dickson, R. Rademakers, J. D. Fryer, B. K. Rush, O. Pedraza, A. M. Caputo, P. Desaro, C. Palmucci, A. Robertson, M. G. Heckman, N. N. Diehl, E. Wiggs, M. Tierney, L. Braun, J. Farren, D. Lacomis, S. Ladha, C. N. Fournier, L. F. McCluskey, L. B. Elman, J. B. Toledo, J. D. McBride, C. Tiloca, C. Morelli, B. Poletti, F. Solca, A. Prelle, J. Wuu, J. Jockel-Balsarotti, F. Rigo, C. Ambrose, A. Datta, W. Yang, D. Raitcheva, G. Antognetti, A. McCampbell, J. C. Van Swieten, B. L. Miller, A. L. Boxer, R. H. Brown, R. Bowser, T. M. Miller, J. Q. Trojanowski, M. Grossman, J. D. Berry, W. T. Hu, A. Ratti, B. J. Traynor, M. D. Disney, M. Benatar, V. Silani, J. D. Glass, M. K. Floeter, J. D. Rothstein, K. B. Boylan, L. Petrucelli, Poly(GP) proteins are a useful pharmacodynamic marker for C9ORF72-associated amyotrophic lateral sclerosis. Sci Transl. Med. 9, eaai7866 (2017).
26. G. Johannes, M. S. Carter, M. B. Eisen, P. O. Brown, P. Sarnow, Identification of eukaryotic mRNAs that are translated at reduced cap binding complex eIF4F concentrations using a cDNA microarray. Proc. Natl. Acad. Sci. U.S.A. 96, 13118-13123 (1999).
27. Q. Zong, M. Schummer, L. Hood, D. R. Morris, Messenger RNA translation state: the second dimension of high-throughput expression screening. Proc. Natl. Acad. Sci. U S. A. 96, 10632-10636 (1999).
28. L. J. Sznajder, J. D. Thomas, E. M. Carrell, T. Reid, K. N. McFarland, J. D. Cleary, R. Oliveira, C. A. Nutter, K. Bhatt, K. Sobczak, T. Ashizawa, C. A. Thornton, L. P. W. Ranum, M. S. Swanson, Intron retention induced by microsatellite expansions as a disease biomarker. Proc. Natl. Acad. Sci. U.S.A. 115, 4234 (2018).
29. S. B. Yamada, T. F. Gendron, T. Niccoli, N. R. Genuth, R. Grosely, Y. Shi, I. Glaria, N. J. Kramer, L. Nakayama, S. Fang, T. J. I. Dinger, A. Thoeng, G. Rocha, M. Barna, J. D. Puglisi, L. Partridge, J. K. Ichida, A. M. Isaacs, L. Petrucelli, A. D. Gitler, RPS25 is required for efficient RAN translation of C9orf72 and other neurodegenerative disease-associated nucleotide repeats. Nat. Neurosci. 22, 1383-1388 (2019).
30. W. Y. Yang, F. He, R. L. Strack, S. Y. Oh, M. Frazer, S. R. Jaffrey, P. K. Todd, M. D. Disney, Small molecule recognition and tools to study modulation of r(CGG)^{exp} in fragile X-associated tremor ataxia syndrome. ACS Chem. Biol. 11, 2456-2465 (2016).
31. M. G. Costales, Y. Matsumoto, S. P. Velagapudi, M. D. Disney, Small molecule targeted recruitment of a nuclease to RNA. J. Am. Chem. Soc. 140, 6741-6744 (2018).
32. M. G. Costales, B. Suresh, K. Vishnu, M. D. Disney, Targeted degradation of a hypoxia-associated non-coding RNA enhances the selectivity of a small molecule interacting with RNA. Cell Chem. Biol. 26, 1180-1186.e1185 (2019).
33. C. S. Thakur, B. K. Jha, B. Dong, J. Das Gupta, K. M. Silverman, H. Mao, H. Sawai, A. O. Nakamura, A. K. Banerjee, A. Gudkov, R. H. Silverman, Small-molecule activators of RNase L with broad-spectrum antiviral activity. Proc. Natl. Acad. Sci. U.S.A. 104, 9585-9590 (2007).
34. Y. Han, J. Donovan, S. Rath, G. Whitney, A. Chitrakar, A. Korennykh, Structure of human RNase L reveals the basis for regulated RNA decay in the IFN response. Science 343, 1244-1248 (2014).
35. D. Sareen, J. G. O'Rourke, P. Meera, A. K. Muhammad, S. Grant, M. Simpkinson, S. Bell, S. Carmona, L. Omelas, A. Sahabian, T. Gendron, L. Petrucelli, M. Baughn, J. Ravits, M. B. Harms, F. Rigo, C. F. Bennett, T. S. Otis, C. N. Svendsen, R. H. Baloh, Targeting RNA foci in iPSC-derived motor neurons from ALS patients with a C9ORF72 repeat expansion. Sci. Transl. Med. 5, 208ra149 (2013).
36. Y. Liu, A. Pattamatta, T. Zu, T. Reid, O. Bardhi, D. R. Borchelt, A. T. Yachnis, L. P. Ranum, C9orf72 BAC mouse model with motor deficits and neurodegenerative features of ALS/FTD. Neuron 90, 521-534 (2016).
37. I. R. Mackenzie, P. Frick, M. Neumann, The neuropathology associated with repeat expansions in the C9ORF72 gene. Acta Neuropathol. 127, 347-357 (2014).
38. J. D. Cleary, L. P. Ranum, Repeat associated non-ATG (RAN) translation: new starts in microsatellite expansion disorders. Curr. Opin. Genet. Dev. 26, 6-15 (2014).
39. G. V. Echeverria, T. A. Cooper, RNA-binding proteins in microsatellite expansion disorders: mediators of RNA toxicity. Brain Res. 1462, 100-111 (2012).
40. A. Mohan, M. Goodwin, M. S. Swanson, RNA-protein interactions in unstable microsatellite diseases. Brain Res. 1584, 3-14 (2014).
41. R. Nalavade, N. Griesche, D. P. Ryan, S. Hildebrand, S. Krauss, Mechanisms of RNA-induced toxicity in CAG repeat disorders. Cell Death Dis. 4, e752 (2013).
42. L. P. Ranum, T. A. Cooper, RNA-mediated neuromuscular disorders. Annu. Rev. Neurosci. 29, 259-277 (2006).
43. M. L. Stephenson, P. C. Zamecnik, Inhibition of Rous sarcoma viral RNA translation by a specific oligodeoxyribonucleotide. Proc. Natl. Acad. Sci. U.S.A 75, 285-288 (1978).
44. T. M. Wheeler, K. Sobczak, J. D. Lueck, R. J. Osborne, X. Lin, R. T. Dirksen, C. A. Thornton, Reversal of RNA dominance by displacement of protein sequestered on triplet repeat RNA. Science 325, 336-339 (2009).
45. J. Hu, M. Matsui, K. T. Gagnon, J. C. Schwartz, S. Gabillet, K. Arar, J. Wu, I. Bezprozvanny, D. R. Corey, Allele-specific silencing of mutant huntingtin and ataxin-3 genes by targeting expanded CAG repeats in mRNAs. Nat. Biotechnol. 27, 478-484 (2009).
46. A. R. La Spada, J. P. Taylor, Repeat expansion disease: progress and puzzles in disease pathogenesis. Nat. Rev. Genet. 11, 247-258 (2010).
47. A. J. Angelbello, S. G. Rzuczek, K. K. McKee, J. L. Chen, H. Olafson, M. D. Cameron, W. N. Moss, E. T. Wang, M. D. Disney, Precise small-molecule cleavage of an r(CUG) repeat expansion in a myotonic dystrophy mouse model. Proc. Natl. Acad. Sci. U.S.A 116, 7799-7804 (2019).
48. K. Sobczak, M. de Mezer, G. Michlewski, J. Krol, W. J. Krzyzosiak, RNA structure of trinucleotide repeats associated with human neurological diseases. Nucleic Acids Res. 31, 5469-5482 (2003).
49. G. Michlewski, W. J. Krzyzosiak, Molecular architecture of CAG repeats in human disease related transcripts. J. Mol. Biol. 340, 665-679 (2004).
50. S. Busan, K. M. Weeks, Role of context in RNA structure: flanking sequences reconfigure CAG motif folding in huntingtin exon 1 transcripts. Biochemistry 52, 8219-8225 (2013).
51. D. H. Mathews, M. D. Disney, J. L. Childs, S. J. Schroeder, M. Zuker, D. H. Turner, Incorporating chemical modification constraints into a dynamic programming algorithm for prediction of RNA secondary structure. Proc. Natl. Acad. Sci. U.S.A 101, 7287-7292 (2004).
52. D. H. Mathews, J. Sabina, M. Zuker, D. H. Turner, Expanded sequence dependence of thermodynamic parameters improves prediction of RNA secondary structure. J Mol. Biol. 288, 911-940 (1999).
53. T. L. Hwang, A. J. Shaka, Water suppression that works. Excitation sculpting using arbitrary wave-forms and pulsed-field gradients. J. Magn. Reson. 112, 275-279 (1995).
54. S. Grzesiek, A. Bax, The importance of not saturating H₂O in protein NMR. Application to sensitivity enhancement and NOE measurements. J. Am. Chem. Soc. 115, 12593-12594 (1993).
55. M. Piotto, V. Saudek, V. Sklenif, Gradient-tailored excitation for single-quantum NMR spectroscopy of aqueous solutions. J. Biomol. NMR 2, 661-665 (1992).
56. T. D. Goddard, D. G. Kneller. (University of California, San Francisco, San Francisco, 2004).
57. A. J. Angelbello, M. D. Disney, Bleomycin can cleave an oncogenic noncoding RNA. Chembiochem 19, 43-47 (2018).
58. A. N. Coyne, B. L. Zaepfel, L. Hayes, B. Fitchman, Y. Salzberg, K. Bowen, H. Trost, F. Rigo, A. Harel, C. N. Svendsen, D. Sareen, J. D. Rothstein, G₄C₂ repeat RNA mediates the disassembly of the nuclear pore complex in C9orf72 ALS/FTD. bioRxiv, doi: 10.1101/2020.02.13.947721 (2020).

### REFERENCES FOR EXPERIMENTAL SECTION

1. J. Jiang, Q. Zhu, T. F. Gendron, S. Saberi, M. McAlonis-Downes, A. Seelman, J. E. Stauffer, P. Jafar-Nejad, K. Drenner, D. Schulte, S. Chun, S. Sun, S. C. Ling, B. Myers, J. Engelhardt, M. Katz, M. Baughn, O. Platoshyn, M. Marsala, A. Watt, C. J. Heyser, M. C. Ard, L. De Muynck, L. M. Daughrity, D. A. Swing, L. Tessarollo, C. J. Jung, A. Delpoux, D. T. Utzschneider, S. M. Hedrick, P. J. de Jong, D. Edbauer, P. Van Damme, L. Petrucelli, C. E. Shaw, C. F. Bennett, S. Da Cruz, J. Ravits, F. Rigo, D. W. Cleveland, C. Lagier-Tourenne, Gain of toxicity from ALS/FTD-linked repeat expansions in C9ORF72 Is alleviated by antisense oligonucleotides targeting GGGGCC-containing RNAs. Neuron 90, 535-550 (2016).
2. C. J. Donnelly, P. W. Zhang, J. T. Pham, A. R. Haeusler, N. A. Mistry, S. Vidensky, E. L. Daley, E. M. Poth, B. Hoover, D. M. Fines, N. Maragakis, P. J. Tienari, L. Petrucelli, B. J. Traynor, J. Wang, F. Rigo, C. F. Bennett, S. Blackshaw, R. Sattler, J. D. Rothstein, RNA toxicity from the ALS/FTD C9ORF72 expansion is mitigated by antisense intervention. Neuron 80, 415-428 (2013).
3. T. F. Gendron, J. Chew, J. N. Stankowski, L. R. Hayes, Y. J. Zhang, M. Prudencio, Y. Carlomagno, L. M. Daughrity, K. Jansen-West, E. A. Perkerson, A. O'Raw, C. Cook, L. Pregent, V. Belzil, M. van Blitterswijk, L. J. Tabassian, C. W. Lee, M. Yue, J. Tong, Y. Song, M. Castanedes-Casey, L. Rousseau, V. Phillips, D. W. Dickson, R. Rademakers, J. D. Fryer, B. K. Rush, O. Pedraza, A. M. Caputo, P. Desaro, C. Palmucci, A. Robertson, M. G. Heckman, N. N. Diehl, E. Wiggs, M. Tierney, L. Braun, J. Farren, D. Lacomis, S. Ladha, C. N. Fournier, L. F. McCluskey, L. B. Elman, J. B. Toledo, J. D. McBride, C. Tiloca, C. Morelli, B. Poletti, F. Solca, A. Prelle, J. Wuu, J. Jockel-Balsarotti, F. Rigo, C. Ambrose, A. Datta, W. Yang, D. Raitcheva, G. Antognetti, A. McCampbell, J. C. Van Swieten, B. L. Miller, A. L. Boxer, R. H. Brown, R. Bowser, T. M. Miller, J. Q. Trojanowski, M. Grossman, J. D. Berry, W. T. Hu, A. Ratti, B. J. Traynor, M. D. Disney, M. Benatar, V. Silani, J. D. Glass, M. K. Floeter, J. D. Rothstein, K. B. Boylan, L. Petrucelli, Poly(GP) proteins are a useful pharmacodynamic marker for C9ORF72-associated amyotrophic lateral sclerosis. Sci. Transl. Med. 9, eaai7866 (2017).
4. C. Lagier-Tourenne, M. Baughn, F. Rigo, S. Sun, P. Liu, H. R. Li, J. Jiang, A. T. Watt, S. Chun, M. Katz, J. Qiu, Y. Sun, S. C. Ling, Q. Zhu, M. Polymenidou, K. Drenner, J. W. Artates, M. McAlonis-Downes, S. Markmiller, K. R. Hutt, D. P. Pizzo, J. Cady, M. B. Harms, R. H. Baloh, S. R. Vandenberg, G. W. Yeo, X. D. Fu, C. F. Bennett, D. W. Cleveland, J. Ravits, Targeted degradation of sense and antisense C9orf72 RNA foci as therapy for ALS and frontotemporal degeneration. Proc. Natl. Acad. Sci. U.S.A 110, E4530-4539 (2013).
5. Z. F. Wang, A. Ursu, J. L. Childs-Disney, R. Guertler, W. Y. Yang, V. Bernat, S. G. Rzuczek, R. Fuerst, Y. J. Zhang, T. F. Gendron, I. Yildirim, B. G. Dwyer, J. E. Rice, L. Petrucelli, M. D. Disney, The hairpin form of r(G₄C₂)^{exp} in c9ALS/FTD is repeat-associated non-ATG translated and a target for bioactive small molecules. Cell Chem. Biol. 26, 179-190.e112 (2019).
6. J. L. Childs-Disney, I. Yildirim, H. Park, J. R. Lohman, L. Guan, T. Tran, P. Sarkar, G. C. Schatz, M. D. Disney, Structure of the myotonic dystrophy type 2 RNA and designed small molecules that reduce toxicity. ACS Chem. Biol. 9, 538-550 (2014).
7. J. L. Childs-Disney, E. Stepniak-Konieczna, T. Tuan, I. Yildirim, H. Park, C. Z. Chen, J. Hoskins, N. Southall, J. J. Marugan, S. Patnaik, W. Zheng, C. P. Austin, G. C. Schatz, K. Sobczak, C. A. Thornton, M. D. Disney, Induction and reversal of myotonic dystrophy type 1 pre-mRNA splicing defects by small molecules. Nat. Commun. 4, 2044 (2013).
8. M. G. Costales, H. Aikawa, Y. Li, J. L. Childs-Disney, D. Abegg, D. G. Hoch, S. Pradeep Velagapudi, Y. Nakai, T. Khan, K. W. Wang, I. Yildirim, A. Adibekian, E. T. Wang, M. D. Disney, Small-molecule targeted recruitment of a nuclease to cleave an oncogenic RNA in a mouse model of metastatic cancer. Proc. Natl. Acad. Sci. U.S.A 117, 2406-2411 (2020).
9. J. M. Wang, R. M. Wolf, J. W. Caldwell, P. A. Kollman, D. A. Case, Development and testing of a general AMBER force field J Comput. Chem. 25, 1157-1174 (2004).
10. C. I. Bayly, P. Cieplak, W. D. Cornell, P. A. Kollman, A well-behaved electrostatic potential based method using charge restraints for deriving atomic charges—the Resp Model. J. Phys. Chem. 97, 10269-10280 (1993).
11. W. D. Cornell, P. Cieplak, C. I. Bayly, P. A. Kollman, Application of RESP charges to calculate conformational energies, hydrogen-bond energies, and free-energies of solvation. J. Am. Chem. Soc. 115, 9620-9631 (1993).
12. M. J. Frisch, G. W. Trucks, H. B. Schlegel, G. E. Scuseria, M. A. Robb, J. R. Cheeseman, G. Scalmani, V. Barone, G. A. Petersson, H. Nakatsuji, X. Li, M. Caricato, A. Marenich, J. Bloino, B. G. Janesko, R. Gomperts, B. Mennucci, H. P. Hratchian, J. V. Ortiz, A. F. Izmaylov, J. L. Sonnenberg, D. Williams-Young, F. Ding, F. Lipparini, F. Egidi, J. Goings, B. Peng, A. Petrone, T. Henderson, D. Ranasinghe, V. G. Zakrzewski, J. Gao, N. Rega, G. Zheng, W. Liang, M. Hada, M. Ehara, K. Toyota, R. Fukuda, J. Hasegawa, M. Ishida, T. Nakajima, Y. Honda, O. Kitao, H. Nakai, T. Vreven, K. Throssell, J. Montgomery, J. A., J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, J. M. Millam, M. Klene, C. Adamo, R. Cammi, J. W. Ochterski, R. L. Martin, K. Morokuma, O. Farkas, J. B. Foresman, D. J. Fox. (Gaussian, Inc., Wallingford, C T, 2016).
13. M. J. L. Sangster, R. M. Atwood, Interionic potentials for alkali halides. II. Completely crystal independent specification of Born-Mayer potentials. J. Phys. C 11, 1541-1555 (1978).
14. I. T. E. Cheatham, J. L. Miller, T. Fox, T. A. Darden, and P. A. Kollman, Molecular Dynamics simulations on solvated biomolecular systems: the particle mesh Ewald method leads to stable trajectories of DNA, RNA, and proteins. J. Am. Chem. Soc. 117, 4193-4194 (1995).
15. E. H. Bednářiová, S.; Jindrich, J., Optimized methods for preparation of 6I-(ω-sulfanyl-alkylene-sulfanyl)-β-cyclodextrin derivatives. Beilstein J. Org. Chem. 12, 349-352 (2016).
16. M. Shan, A. Bujotzek, F. Abendroth, A. Wellner, R. Gust, O. Seitz, M. Weber, R. Haag, Conformational analysis of bivalent estrogen receptor ligands: from intramolecular to intermolecular binding. Chembiochem 12, 2587-2598 (2011).
17. Y. Cao, J. Yang, Development of a folate receptor (FR)-targeted indenoisoquinoline using a pH-sensitive N-ethoxybenzylimidazole (NEBI) bifunctional cross-linker. Bioconjug. Chem. 25, 873-878 (2014).
18. M. Dracinský, J. Sejbal, B. Rygerová, M. Stiborová, An efficient modification of ellipticine synthesis and preparation of 13-hydroxyellipticine. Tetrahedron Lett. 48, 6893-6895 (2007).
19. C. P. Mandl, B. König, Synthesis of mono protected 1,10-diaza-18-crown-6. Synth. Commun. 34, 3573-3578 (2004).
20. C. P. M. B. Konig, Synthesis of mono protected 1,10-diaza-18-crown-6. Synth. Commun. 34, 3573-3578 (2004).

### SUMMARY STATEMENTS

As the biology underpinning the pathology of microsatellite diseases has unfolded, it has become clear that many mechanisms contribute to toxicity. These include the binding of RNA-binding proteins (RBPs) to repeat RNA resulting in their sequestration, loss of function, and the production of toxic proteins translated from the expanded repeats (28, 38-42).

Embodiments of the present invention provide therapeutic treatment of a certain kind of such microsatellite diseases, amyotrophic lateral sclerosis and frontotemporal dementia due to their ability to bind the repeat RNA. These embodiments for therapeutic treatment are based upon the ALS compounds described in this specification. Especially, this therapeutic treatment is accomplished by the ALS compounds which are amino bis-polyoxyethylenyl bridging dimers of the pyridocarbazole moiety. These ALS compounds alleviate c9ALS/FTD-associated defects at the RNA, and protein level in patient-derived LCLs, iPSCs, and iPSNs.

Further, the ALS compound including the Rnase L-recruiting moiety (ALS compound) similarly mitigates c9ALS/FTD-associated features in a BAC transgenic mouse model. The mode of action of the first ALS compound (3, ALS compound of Formula II, Y is N—H) is to selectively bind the structure formed by r(G₄C₂)^{exp}, thereby inhibiting the binding of RBPs to r(G₄C₂)^{exp}, and the loading of ribosomes that aberrantly translate the repeat expansion into DPRs. This ALS compound 3 (ALS compound of Formula II, Y is N—H) alleviated c9ALS/FTD-associated features in patient-derived cells at nM concentrations.

The second ALS compound (7, ALS compound of Formulas II and III) selectively targets the unique structure of r(G₄C₂)^{exp} for degradation by recruiting the endogenous nuclease Rnase L. The mode of action of this ALS compound (7) is the selective recognition and cleavage of the mutant C9orf72 allele, while causing no significant changes in the levels of WT C9orf72 transcripts or other transcripts containing short, nonpathogenic r(G₄C₂) repeats (FIGS. 5-F & 12-G). Further, this ALS compound (7) inhibits RAN translation of r(G₄C₂)^{exp}, thus reducing levels of the DPR Poly(GP) without affecting expression of WT C9ORF72 protein expression.² In vivo 7 reduced the abundance of DPR aggregates Poly(GP) and Poly(GA) in the cortex. Importantly, the ALS compound of Formulas II and III rescued c9ALS/FTD-associated pathological hallmarks in patient-derived lymphoblastoid cells (FIG. 5A), iPSCs (FIGS. 5-G & H), differentiated iPSNs (including by RNA-seq analysis; FIG. 5, I-K; and FIG. 12, E) and in vivo (FIGS. 6 & 15). The selectivity of 7, like 3, is the result of its recognition of the structure formed by r(G₄C₂)^{exp} rather than its sequence. ² This ALS compound (7) did so more potently than ALS compound without the RNase recruiting moiety (3).

### MISCELLANEOUS STATEMENTS

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The invention has been described broadly and generically herein. Each of the narrower species and subgeneric groupings falling within the generic disclosure also form part of the invention. This includes the generic description of the invention with a proviso or negative limitation removing any patient matter from the genus, regardless of whether or not the excised material is specifically recited herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

All references cited herein are incorporated herein by reference as if fully set forth herein in their entirety.

What is claimed is:

1. A method comprising contacting a hexanucleotide repeat expansion RNA r(G₄C₂)^{exp} with an ALS compound wherein the ALS compound comprises a pyridocarbazole moiety bound to X according to Formula I 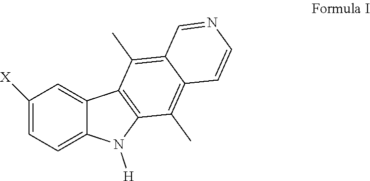
wherein:
X is hydrogen, hydroxyl, C₁ to C₄ alkoxy; or
X is a bridging polyoxyethylenyl or a bridging R-amino-bispolyoxyethylenyl group having the pyridocarbazole moiety covalently attached to each of its termini to comprise a bridged dimer of the pyridocarbazole moiety,
R-amino- is R—N— with R as hydrogen, acetyl, an Rnase recruiting moiety or a diaza-4,4′-oct-7yn-1-oyl group;
and a pharmaceutically acceptable salt thereof.

2. A method according to claim 1 wherein the contacting binds and/or complexes the r(G₄C₂)^{exp}.

3. A method according to claim 1 wherein the ALS compound is the bridged dimer and the bridged dimer comprises Formula II 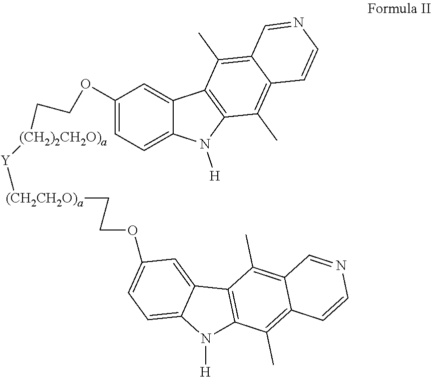
wherein a is an integer of 1 to 5, preferably 2 to 5, more preferably 2 to 3, especially more preferably 2, Y is oxygen or R—N wherein R comprises hydrogen, acetyl, an Rnase L-recruiting moiety bound to N comprising Formula III, a diaza-4,4′-oct-7yn-1-oyl group bound to N comprising Formula IV or a succinoyl group comprising Formula V:

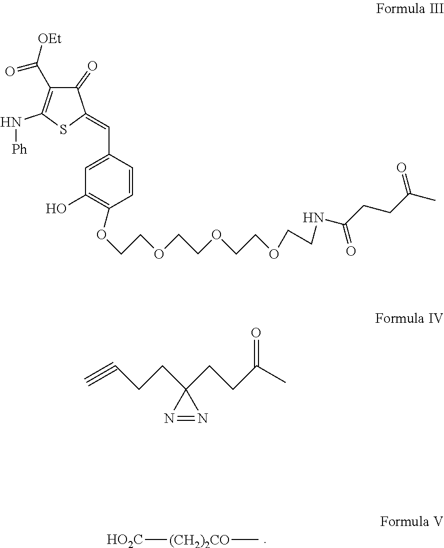

4. A method according to claim 3 wherein Y is Formula III and a is 2 or 3, preferably 2.

5. A method according to any of claims 1-4 wherein the r(G₄C₂)^{exp} is r(G₄C₂)ₘ wherein m is at least 20.

6. A method according to claim 5 wherein r(G₄C₂)ₘ is an abnormal number of repeats with m being at least 20-1000.

7. A method according to any of claims 1-6 wherein the r(G₄C₂)^{exp} is a repeat RNA hairpin structure.

8. A method according to any of claims 1-7 wherein r(G₄C₂)^{exp} is present in a cell.

9. A method according to claim 8 wherein Y is R—N and R bound to N is hydrogen or Formula III.

10. A method according to claim 8 or 9 wherein the cell contains chromosome 9 open reading frame 72 (C9orf72) and r(G₄C₂)^{exp} is present in the intron 1 of C9orf72.

11. A method according to any of claim 9 or 10 wherein the cells are patient-derived cells.

12. A method of any of claims 9-11 wherein the cells are incubated with the ALS compound of claim 2 with Y as Formula III.

13. A method according to claim 12 wherein the cells are HEK293T cells, patient-derived lymphoblastoid cells, induced pluripotent stem cells (c9 iPSCs cells), iPSC-derived spinal neurons (c9 iPSNs).

14. A method according to claim 12 wherein the cells are c9ALS/FTD BAC cells in a transgenic mouse model.

15. A method according to any of claims 12-14 wherein the ALS compound decreases RAN translation of r(G₄C₂)^{exp}.

16. A method according to claim 15 wherein the ALS compound inhibits RAN translation of r(G₄C₂)^{exp}.

17. A method according to claim 15 or 16 wherein the ALS compound does not inhibit transcription of C9orf72.

18. A method according to claims 12-14 wherein the ALS compound decreases the number of nuclear foci.

19. A method according to claims 12-14 wherein the ALS compound alleviates defects in nuclear trafficking.

20. A method according to claims 12-14 wherein the ALS compound facilitates degradation of the repeat expansion.

21. A method according to claim 13 wherein the HEK293T cells are cotransfected with a plasmid expressing (G₄C₂)₆₆-NOATG-Nano-luciferase or SV40-Firefly luciferase.

22. A pharmaceutical composition comprising a pharmaceutically acceptable carrier and an ALS compound according to Formula II of claim 3.

23. A pharmaceutical composition according to claim 22 wherein the ALS compound comprises Formulas II and III of claim 3.

24. A pharmaceutical composition according to claim 23 wherein Formula III has designation a as 2.

25. A pharmaceutical composition according to any of claims 22-24 wherein the pharmaceutically acceptable carrier comprises excipients suitable for a selected route of administration of the ALS compound.

26. A pharmaceutical composition according to any of claims 22-25 wherein the amount of ALS compound provides an effective dose of the ALS compound for treatment of ALS/FTD disease.

27. A method for treatment of an ALS/FTD disease comprising administration to a patient having the disease, an effective amount of an ALS compound comprising the dimer of Formulas II and III of claim 3.

28. A method for treatment of an ALS/FTD disease comprising administration to a patient having the disease, a pharmaceutical composition of any of claims 22-26.

29. A method for treatment according to claim 28 wherein the ALS compound of the pharmaceutical composition comprises Formulas II and III of claim 3.

30. A method for treatment according to any of claims 27-29 wherein the ALS/FTD disease is amyotrophic lateral sclerosis.

31. A method according to claim 30 wherein the administration step comprises oral, intramuscular, intravenous or intrathecal administration of the ALS compound in a pharmaceutically acceptable medium.

32. A method according to claim 31 wherein the ALS compound in a pharmaceutically acceptable medium is a pharmaceutical composition.

33. A method according to claim 32 wherein the pharmaceutical composition comprises pharmaceutically acceptable excipients suitable for a selected route of administration and the excipients are compatible with the ALS compound.

34. A composition comprising a bis bridged pyridocarbazole of Formula II and a pharmaceutically acceptable salt thereof: 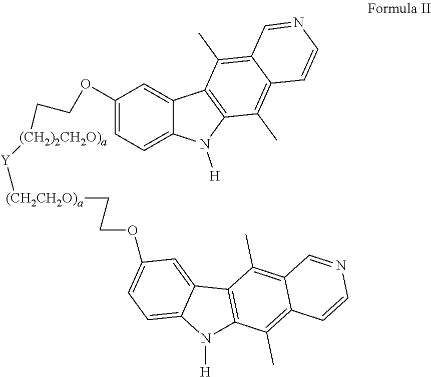
wherein a is an integer of 2 to 5, Y is oxygen or R—N wherein R comprises hydrogen, acetyl, an Rnase recruiting moiety bound to N comprising Formula III or a diaza-4,4′-oct-7-yn-1-oyl group bound to N comprising Formula IV or a succinoyl group comprising Formula V:

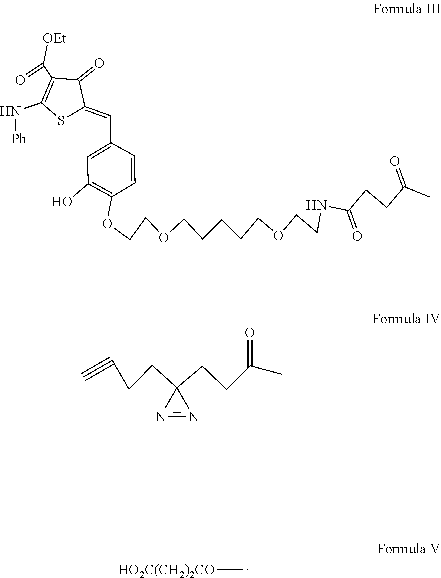

35. A composition according to claim 34 wherein Y is Formula III.
